(19) United States
(12) Patent Application Publication
(10) Pub. No.: US 2025/0075185 A1
(43) Pub. Date: Mar. 6, 2025

(54) Orthopedic Regeneration by Inducible Pluripotent Stem Cell Derived Mesenchymal Stem Cells

(71) Applicant: CREATIVE MEDICAL TECHNOLOGIES, INC., Phoenix, AZ (US)

(72) Inventors: Thomas Ichim, San Diego, CA (US); Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/785,305

(22) Filed: Jul. 26, 2024

Related U.S. Application Data
(60) Provisional application No. 63/580,669, filed on Sep. 5, 2023.

(51) Int. Cl. C12N 5/0775; A61K 31/198; A61K 35/28

(52) U.S. Cl. CPC C12N 5/0665; A61K 31/198; A61K 35/28; C12N 2500/02; C12N 2510/00

(57) ABSTRACT

Compositions of matter and therapeutic means for stimulation bone, cartilage or joint regeneration by pluripotent stem cell derived mesenchymal stem cells alone or in a manipulated manner. “Semidifferentiated” mesenchymal stem cells which possess enhanced ability to generate cartilage, bone, or joint tissue. Gene editing is utilized to generate cells with reduced immunogenicity and enhanced therapeutic potential. Enhancement of therapeutic activity is achieved by transfection of pluripotent stem cells with genes which inhibit apoptosis, thus enhancing lifespan and therapeutic activity of said stem cells. Transfection with antioxidant genes such as superoxide dismutase is utilized to enhance activity of cells when administered in inflammatory environments.

### CROSS-REFERENCE TO RELATED APPLICATIONS

The present application claims benefit of U.S. Provisional Patent Application Ser. No. 63/580,669, filed on Sep. 5, 2023, titled ORTHOPEDIC REGENERATION BY INDUCIBLE PLURIPOTENT STEM CELL DERIVED MESENCHYMAL STEM CELLS, the contents of which are incorporated herein by reference in its entirety.

### FIELD OF THE INVENTION

The invention pertains to the field of generating cellular therapeutics for the treatment of orthopedic disorders.

### BACKGROUND OF THE INVENTION

As part of the musculoskeletal system, it is known that cartilage acts as the structural basis of several organs and systems. This includes the articular surface of joints and other joint-associated structures, including the ear, the nose, the larynx, the trachea, the bronchi, structures of the heart valves, etc.

Mammals possess several different forms of cartilage, specifically: a) fibro-cartilage; b) elastic cartilage and c) hyaline cartilage.

Fibro-cartilage contains an abundance of type I collagen and is found in the intervertebral disks and ligaments.

Elastic cartilage contains elastin fibrils and is found in the pinna of the ear and in the epiglottis.

Hyaline cartilage, a semi-transparent and clear cartilage tissue found in the iarthrodial walls of the trachea and bronchia, the costal cartilage and growth plate, as well as in cartilage of the nose, larynx and iarthrodial joints, contains neither type I collagen nor elastin. Hyaline cartilage having a distinctive combination of cartilage-specific collagens (types II, VI, IX, and XI) and aggregating proteoglycans (aggrecan) that give it the unique ability to withstand compressive forces is called articular cartilage.

It is known that damage to articular cartilage results in lesions of the joint surface, and progressive degeneration of these lesions often leads to symptomatic joint pain, disability and reduced or disturbed functionality. Joint surface defects can be the result of various aetiologies, including inflammatory processes, neoplasias, post-traumatic and degenerative events, etc. Adult articular cartilage has a major shortcoming: unlike most tissues, it cannot repair itself. Lack of a blood supply in large part restricts the tissue's ability to recruit chondroprogenitor cells that can act to repair articular cartilage defects. Consequently, articular cartilage defects that have progressed to advanced degenerative disease require total joint arthroplasty to eliminate pain and to restore normal joint function.

Numerous attempts are being made at generating artificial articular cartilage through tissue engineering. This is a biologic solution which may delay or reduce the need for metal- and polymer-based materials currently used in total joint arthroplasty.

For the treatment of cartilage injuries, one technique that has gained FDA approval is a procedure called Autologous Chondrocyte Implantation (Carticel, Genzyme Surgery). In this protocol, a small tissue biopsy obtained from the patient's joint articular cartilage is taken to the lab where chondrocytes (cartilage cells) are isolated and expanded ex vivo for subsequent re-implantation into the patient in a second surgical procedure. A key limitation of this method is the relatively small number of donor cells that can be obtained at biopsy, and chondrocytes derived from adult articular cartilage appear to have a limited ability to produce cartilage matrix after expansion.

Other areas of interest for treatment of orthopedic conditions include healing of bone injuries. Currently non-union bone fractures, and non-healing fractures in diabetes pose a significant burden on our health care system. In both of these areas failure of healing is associated with reduced or absent progenitor cell numbers.

The invention aims to generate cellular therapies for orthopedic conditions involving cartilage and bone through generation of stem cells and progenitor cells from pluripotent stem cells that possess enhanced features including ability to produce growth factors, ability to deposit extracellular matrix, ability to differentiate and ability to provide cells with reduced or absent immunogenicity.

### SUMMARY OF THE INVENTION

Preferred embodiments are directed to a mesenchymal stem cell useful for the treatment of orthopedic conditions, wherein said mesenchymal stem cell is: a) generated from a pluripotent stem cell; b) possess enhanced regenerative activity; and c) optionally possesses enhanced antioxidant activity.

Preferred methods include embodiments wherein said orthopedic condition is selected from a group of conditions comprising: a) bone fracture; b) non-union bone fracture; c) osteoarthritis; d) rheumatoid arthritis; e) cartilage degeneration; f) torn meniscus; and g) degenerative disc disease.

Preferred methods include embodiments wherein said osteoarthritis is associated with increased expression of MMP-1.

Preferred methods include embodiments wherein said osteoarthritis is associated with increased expression of MMP-3.

Preferred mesenchymal stem cells include embodiments wherein said osteoarthritis is associated with increased expression of MMP-7.

Preferred mesenchymal stem cells include embodiments wherein said osteoarthritis is associated with increased expression of MMP-9.

Preferred mesenchymal stem cells include embodiments wherein said osteoarthritis is associated with increased expression of MMP-13.

Preferred mesenchymal stem cells include embodiments wherein said osteoarthritis is associated with decreased expression of TIMP-1.

Preferred mesenchymal stem cells include embodiments wherein said osteoarthritis is associated with decreased expression of TIMP-3.

Preferred mesenchymal stem cells include embodiments wherein said osteoarthritis is associated with decreased expression of TIMP-5.

Preferred mesenchymal stem cells include embodiments wherein said osteoarthritis is associated with neutrophilic infiltration.

Preferred mesenchymal stem cells include embodiments wherein said osteoarthritis is associated with monocytic infiltration.

Preferred mesenchymal stem cells include embodiments wherein said osteoarthritis is associated with complement activation.

Preferred mesenchymal stem cells include embodiments wherein said osteoarthritis is associated with reduction in aggrecan content of cartilage.

Preferred mesenchymal stem cells include embodiments wherein said osteoarthritis is associated with reduction in collagen content of cartilage.

Preferred mesenchymal stem cells include embodiments wherein said cell is generated by culture of pluripotent stem cells in a decellularized bone matrix.

Preferred mesenchymal stem cells include embodiments wherein said pluripotent stem cell is first cultured in a suspension culture, wherein said suspension culture allows for said pluripotent stem cells to form embryoid bodies.

Preferred mesenchymal stem cells include embodiments wherein said suspension culture is performed under conditions of hypoxia.

Preferred mesenchymal stem cells include embodiments wherein said hypoxia is performed for a time period and intensity sufficient to allow for nuclear translocation of hypoxia inducible factor.

Preferred mesenchymal stem cells include embodiments wherein said suspension culture is performed in a media containing BMP2.

Preferred mesenchymal stem cells include embodiments wherein said suspension culture is performed in a media containing BMP4.

Preferred mesenchymal stem cells include embodiments wherein said suspension culture is performed in a media containing VEGF.

Preferred mesenchymal stem cells include embodiments wherein said suspension culture is performed in a media containing VEGF-C.

Preferred mesenchymal stem cells include embodiments wherein said suspension culture is performed in a media containing GDF-15.

Preferred mesenchymal stem cells include embodiments wherein said suspension culture is performed in a media containing leukemia inhibitory factor.

Preferred mesenchymal stem cells include embodiments wherein said embryoid bodies are cultured on decellularized bone matrix in a manner to allow for exit of mesenchymal stem cells from said embryoid bodies.

Preferred mesenchymal stem cells include embodiments wherein said mesenchymal stem cells are stimulated to differentiate from said embryoid bodies by treatment with one or more growth factors.

Preferred mesenchymal stem cells include embodiments wherein said growth factor is KGF1.

Preferred mesenchymal stem cells include embodiments wherein said growth factor is HGF1.

Preferred mesenchymal stem cells include embodiments wherein said growth factor is IL-22.

Preferred mesenchymal stem cells include embodiments wherein said growth factor is endoglin.

Preferred mesenchymal stem cells include embodiments wherein said growth factor is LIF-1.

Preferred mesenchymal stem cells include embodiments wherein said growth factor is lymphocyte conditioned media.

Preferred mesenchymal stem cells include embodiments wherein said lymphocyte conditioned media is extracted from a culture of peripheral blood derived mononuclear cells in the presence of a stimulator of cytokine production.

Preferred mesenchymal stem cells include embodiments wherein said stimulator of cytokine production induces nuclear translocation of NF-kappa B.

Preferred mesenchymal stem cells include embodiments wherein said cytokine production is stimulated by exposure of said mononuclear cells to a mitogen.

Preferred mesenchymal stem cells include embodiments wherein said mitogen is a lectin.

Preferred mesenchymal stem cells include embodiments wherein said mitogen is concanavalin A.

Preferred mesenchymal stem cells include embodiments wherein said mitogen is PHA.

Preferred mesenchymal stem cells include embodiments wherein said mitogen is pokeweed mitogen.

Preferred mesenchymal stem cells include embodiments wherein said mitogen is cynavirin.

Preferred mesenchymal stem cells include embodiments wherein said mitogen is lipopolysaccharide.

Preferred mesenchymal stem cells include embodiments wherein said mitogen is HMBG1.

Preferred mesenchymal stem cells include embodiments wherein said mitogen is Poly IC.

Preferred mesenchymal stem cells include embodiments wherein said mitogen is beta glucan.

Preferred mesenchymal stem cells include embodiments wherein said mitogen is CpG DNA.

Preferred mesenchymal stem cells include embodiments wherein said mitogen is allogeneic antigen presenting cells.

Preferred mesenchymal stem cells include embodiments wherein said allogeneic antigen presenting cell is a dendritic cell.

Preferred mesenchymal stem cells include embodiments wherein said allogeneic antigen presenting cell is a B cell.

Preferred mesenchymal stem cells include embodiments wherein said allogeneic antigen presenting cell is a T cell.

Preferred mesenchymal stem cells include embodiments wherein said allogeneic antigen presenting cell is an endothelial cell.

Preferred mesenchymal stem cells include embodiments wherein said allogeneic antigen presenting cell is a fibroblast.

Preferred mesenchymal stem cells include embodiments wherein said growth factor is monocyte conditioned media.

Preferred mesenchymal stem cells include embodiments wherein said monocyte conditioned media is obtained by culturing monocytes in hypoxia.

Preferred mesenchymal stem cells include embodiments wherein said hypoxia is of sufficient length and intensity to stimulate production of 20 ng or more of interleukin-1 receptor antagonist per 1 million cells.

Preferred mesenchymal stem cells include embodiments wherein said hypoxia is of sufficient length and intensity to stimulate production of 40 ng or more of interleukin-1 receptor antagonist per 1 million cells.

Preferred mesenchymal stem cells include embodiments wherein said hypoxia is of sufficient length and intensity to stimulate production of 100 ng or more of interleukin-1 receptor antagonist per 1 million cells.

Preferred mesenchymal stem cells include embodiments wherein said hypoxia is of sufficient length and intensity to stimulate production of 1 ng or more HGF-1 per 1 million cells.

Preferred mesenchymal stem cells include embodiments wherein said hypoxia is of sufficient length and intensity to stimulate production of 5 ng or more HGF-1 per 1 million cells.

Preferred mesenchymal stem cells include embodiments wherein said hypoxia is of sufficient length and intensity to stimulate production of 5 ng or more HGF-1 per 1 million cells.

Preferred mesenchymal stem cells include embodiments wherein said hypoxia is of sufficient length and intensity to stimulate production of 10 pg or more FGF-1 per 1 million cells.

Preferred mesenchymal stem cells include embodiments wherein said hypoxia is of sufficient length and intensity to stimulate production of 50 pg or more FGF-1 per 1 million cells.

Preferred mesenchymal stem cells include embodiments wherein said hypoxia is of sufficient length and intensity to stimulate production of 100 ng or more FGF-1 per 1 million cells.

Preferred mesenchymal stem cells include embodiments wherein said hypoxia is of sufficient length and intensity to stimulate production of 20 pg or more FGF-2 per 1 million cells.

Preferred mesenchymal stem cells include embodiments wherein said hypoxia is of sufficient length and intensity to stimulate production of 40 pg or more FGF-2 per 1 million cells.

Preferred mesenchymal stem cells include embodiments wherein said hypoxia is of sufficient length and intensity to stimulate production of 100 pg or more FGF-2 per 1 million cells.

Preferred mesenchymal stem cells include embodiments wherein said hypoxia is of sufficient length and intensity to stimulate production of 1 ng or more VEGF per 1 million cells.

Preferred mesenchymal stem cells include embodiments wherein said hypoxia is of sufficient length and intensity to stimulate production of 4 ng or more VEGF per 1 million cells.

Preferred mesenchymal stem cells include embodiments wherein said hypoxia is of sufficient length and intensity to stimulate production of 10 ng or more VEGF per 1 million cells.

Preferred mesenchymal stem cells include embodiments wherein said pluripotent stem cell is an embryonic stem cell.

Preferred mesenchymal stem cells include embodiments wherein said pluripotent stem cell is a parthenogenesis derived stem cell.

Preferred mesenchymal stem cells include embodiments wherein said pluripotent stem cell is a somatic cell nuclear transfer derived stem cell.

Preferred mesenchymal stem cells include embodiments wherein said pluripotent stem cell is a stressed induced dedifferentiated stem cell.

Preferred mesenchymal stem cells include embodiments wherein said pluripotent stem cell is a cytoplasm exchange derived stem cell.

Preferred mesenchymal stem cells include embodiments wherein said pluripotent stem cell is a chemical dedifferentiation induced stem cell.

Preferred mesenchymal stem cells include embodiments wherein said pluripotent stem cell is an induced pluripotent stem cell.

Preferred mesenchymal stem cells include embodiments wherein said induced pluripotent stem cell is generated by transfection with genes selected form a group comprising of: a) KLF4; b) sox-2; c) PIM1; d) OCT4; e) NANOG and f) ras.

Preferred mesenchymal stem cells include embodiments wherein said induced pluripotent stem cells are generated from mesenchymal stem cells.

Preferred mesenchymal stem cells include embodiments wherein said mesenchymal stem cell is selected form a group of cells based on expression of CD73.

Preferred mesenchymal stem cells include embodiments wherein said mesenchymal stem cell is selected form a group of cells based on expression of CD37.

Preferred mesenchymal stem cells include embodiments wherein said mesenchymal stem cell is selected form a group of cells based on expression of stem cell antigen.

Preferred mesenchymal stem cells include embodiments wherein said mesenchymal stem cell is selected form a group of cells based on expression of IL-3 receptor.

Preferred mesenchymal stem cells include embodiments wherein said mesenchymal stem cell is selected form a group of cells based on expression of IL-7 receptor.

Preferred mesenchymal stem cells include embodiments wherein said mesenchymal stem cell is selected form a group of cells based on expression of TNF-alpha receptor p55.

Preferred mesenchymal stem cells include embodiments wherein said mesenchymal stem cell is selected form a group of cells based on expression of TNF-alpha receptor p75.

Preferred mesenchymal stem cells include embodiments wherein said mesenchymal stem cell is purified from bone marrow.

Preferred mesenchymal stem cells include embodiments wherein said mesenchymal stem cell is purified from Wharton's Jelly.

Preferred mesenchymal stem cells include embodiments wherein said mesenchymal stem cell is purified from subepithelial umbilical cord tissue.

Preferred mesenchymal stem cells include embodiments wherein said mesenchymal stem cell is purified from adipose tissue.

Preferred mesenchymal stem cells include embodiments wherein said mesenchymal stem cell is purified from perinatal tissue.

Preferred mesenchymal stem cells include embodiments wherein said mesenchymal stem cell is purified from menstrual blood.

Preferred mesenchymal stem cells include embodiments wherein said mesenchymal stem cell is purified from endometrial tissue blood.

Preferred mesenchymal stem cells include embodiments wherein said mesenchymal stem cell is purified from omental tissue.

Preferred mesenchymal stem cells include embodiments wherein said mesenchymal stem cell is purified from deciduous tooth.

Preferred mesenchymal stem cells include embodiments wherein said mesenchymal stem cell is purified from mobilized peripheral blood.

Preferred mesenchymal stem cells include embodiments wherein said peripheral blood is mobilized by treatment with G-CSF.

Preferred mesenchymal stem cells include embodiments wherein said peripheral blood is mobilized by treatment with M-CSF.

Preferred mesenchymal stem cells include embodiments wherein said peripheral blood is mobilized by treatment with GM-CSF.

Preferred mesenchymal stem cells include embodiments wherein said cell is engineered to possess increased expression of superoxide dismutase as compared to a non-engineered mesenchymal stem cell.

Preferred mesenchymal stem cells include embodiments wherein said cell is engineered to possess increased expression of manganese dependent superoxide dismutase as compared to a non-engineered mesenchymal stem cell.

Preferred mesenchymal stem cells include embodiments wherein said cell is engineered to possess increased expression of bone morphogenic protein 2 as compared to a non-engineered mesenchymal stem cell.

Preferred mesenchymal stem cells include embodiments wherein said cell is engineered to possess increased expression of bone morphogenic protein 4 as compared to a non-engineered mesenchymal stem cell.

Preferred mesenchymal stem cells include embodiments wherein said cell is engineered to possess increased expression of VEGF as compared to a non-engineered mesenchymal stem cell.

Preferred mesenchymal stem cells include embodiments wherein said cell is engineered to possess increased expression of VEGF-C as compared to a non-engineered mesenchymal stem cell.

Preferred mesenchymal stem cells include embodiments wherein said cell is engineered to possess increased expression of CXCL12 as compared to a non-engineered mesenchymal stem cell.

Preferred mesenchymal stem cells include embodiments wherein said cell is engineered to possess increased expression of TIMP-1 as compared to a non-engineered mesenchymal stem cell.

Preferred mesenchymal stem cells include embodiments wherein said cell is engineered to possess increased expression of TIMP-3 as compared to a non-engineered mesenchymal stem cell.

Preferred mesenchymal stem cells include embodiments wherein said cell is engineered to possess increased expression of TIMP-5 as compared to a non-engineered mesenchymal stem cell.

Preferred mesenchymal stem cells include embodiments wherein said cell is engineered to possess increased expression of IL-10 as compared to a non-engineered mesenchymal stem cell.

Preferred mesenchymal stem cells include embodiments wherein said cell is engineered to possess increased expression of Fas ligand as compared to a non-engineered mesenchymal stem cell.

Preferred mesenchymal stem cells include embodiments wherein said cell is engineered to possess increased expression of HLA-G as compared to a non-engineered mesenchymal stem cell.

Preferred mesenchymal stem cells include embodiments wherein said cell is engineered to possess increased expression of TIMP-5 as compared to a non-engineered mesenchymal stem cell.

Preferred mesenchymal stem cells include embodiments wherein said cell is utilized to enhance engraftment of a chondrocytic progenitor.

Preferred mesenchymal stem cells include embodiments wherein said chondrocytic progenitor is autologous to the recipient.

Preferred mesenchymal stem cells include embodiments wherein said chondrocytic progenitor is allogeneic to the recipient.

Preferred mesenchymal stem cells include embodiments wherein said chondrocytic progenitor is xenogeneic to the recipient.

Preferred mesenchymal stem cells include embodiments wherein said mesenchymal stem cell is cultured with said chondrocytic progenitor before administration of said chondrocytic progenitor.

Preferred mesenchymal stem cells include embodiments wherein said chondrocytic progenitor is utilized to treat a defect of hyalin cartilage.

Preferred mesenchymal stem cells include embodiments wherein said chondrocytic progenitor is utilized to treat rheumatoid arthritis.

Preferred mesenchymal stem cells include embodiments wherein said chondrocytic progenitor is utilized to treat osteoarthritis.

Preferred mesenchymal stem cells include embodiments wherein said chondrocytic progenitor is utilized to treat disc degenerative disease.

Preferred mesenchymal stem cells include embodiments wherein said chondrocytic progenitor is administered together with FGF-5.

Preferred mesenchymal stem cells include embodiments wherein said cell is utilized to treat a non-union bone fracture.

Preferred mesenchymal stem cells include embodiments wherein said mesenchymal stem cell is administered together with an anti-inflammatory agent.

Preferred mesenchymal stem cells include embodiments wherein said anti-inflammatory agent is capable of inhibiting activation of NF-kappa B.

Preferred mesenchymal stem cells include embodiments wherein said anti-inflammatory agent is capable of inhibiting degradation of i-kappa B.

Preferred mesenchymal stem cells include embodiments wherein said anti-inflammatory agent is n-acetylcysteine.

Preferred mesenchymal stem cells include embodiments wherein said n-acetylcysteine is administered at a concentration sufficient to increase production of IL-10 from mesenchymal stem cells stimulated with HMGB1 by over 25% as compared to baseline.

Preferred mesenchymal stem cells include embodiments wherein said n-acetylcysteine is administered at a concentration sufficient to increase production of IL-10 from mesenchymal stem cells stimulated with HMGB1 by over 50% as compared to baseline.

Preferred mesenchymal stem cells include embodiments wherein said n-acetylcysteine is administered at a concentration sufficient to increase production of IL-10 from mesenchymal stem cells stimulated with HMGB1 by over 100% as compared to baseline.

Preferred mesenchymal stem cells include embodiments wherein said anti-inflammatory agent is quercetin.

Preferred mesenchymal stem cells include embodiments wherein said quercetin is administered at a concentration sufficient to increase production of IL-10 from mesenchymal stem cells stimulated with HMGB1 by over 25% as compared to baseline.

Preferred mesenchymal stem cells include embodiments wherein said quercetin is administered at a concentration sufficient to increase production of IL-10 from mesenchymal stem cells stimulated with HMGB1 by over 50% as compared to baseline.

Preferred mesenchymal stem cells include embodiments wherein said quercetin is administered at a concentration sufficient to increase production of IL-10 from mesenchymal stem cells stimulated with HMGB1 by over 100% as compared to baseline.

Preferred mesenchymal stem cells include embodiments wherein said anti-inflammatory agent is indomethacin.

Preferred mesenchymal stem cells include embodiments wherein said indomethacin is administered at a concentration sufficient to increase production of IL-10 from mesenchymal stem cells stimulated with HMGB1 by over 25% as compared to baseline.

Preferred mesenchymal stem cells include embodiments wherein said indomethacin is administered at a concentration sufficient to increase production of IL-10 from mesenchymal stem cells stimulated with HMGB1 by over 50% as compared to baseline.

Preferred mesenchymal stem cells include embodiments wherein said indomethacin is administered at a concentration sufficient to increase production of IL-10 from mesenchymal stem cells stimulated with HMGB1 by over 100% as compared to baseline.

Preferred mesenchymal stem cells include embodiments wherein said anti-inflammatory agent is ampiroxicam.

Preferred mesenchymal stem cells include embodiments wherein said ampiroxicam is administered at a concentration sufficient to increase production of IL-10 from mesenchymal stem cells stimulated with HMGB1 by over 25% as compared to baseline.

Preferred mesenchymal stem cells include embodiments wherein said ampiroxicam is administered at a concentration sufficient to increase production of IL-10 from mesenchymal stem cells stimulated with HMGB1 by over 50% as compared to baseline.

Preferred mesenchymal stem cells include embodiments wherein said ampiroxicam is administered at a concentration sufficient to increase production of IL-10 from mesenchymal stem cells stimulated with HMGB1 by over 100% as compared to baseline.

Preferred mesenchymal stem cells include embodiments wherein said cell is maintained in an undifferentiated state.

Preferred mesenchymal stem cells include embodiments wherein said maintenance in said undifferentiated state is accomplished by culture in a media conditioned be dedifferentiated fibroblasts.

Preferred mesenchymal stem cells include embodiments wherein said dedifferentiated fibroblasts are fibroblasts transfected with OCT4.

Preferred mesenchymal stem cells include embodiments wherein said dedifferentiated fibroblasts are fibroblasts transfected with NANOG.

Preferred mesenchymal stem cells include embodiments wherein said dedifferentiated fibroblasts are fibroblasts transfected with lin28.

Preferred mesenchymal stem cells include embodiments wherein said dedifferentiated fibroblasts are fibroblasts transfected with one or more proteins selected from a group comprising of: a) PIM1; b) c-myc; c) k-ras; d) bcr-abl; e) KLF4; f) c-met; g) OCT4; h) NANOG; and i) AIRE.

Preferred mesenchymal stem cells include embodiments wherein said dedifferentiated fibroblasts are obtained from a tissue possessing immature properties.

Preferred mesenchymal stem cells include embodiments wherein said fibroblasts are obtained from testicular tissue.

Preferred mesenchymal stem cells include embodiments wherein said fibroblasts are obtained from ovarian tissue.

Preferred mesenchymal stem cells include embodiments wherein said fibroblasts are obtained from placental tissue.

Preferred mesenchymal stem cells include embodiments wherein said fibroblasts are obtained from amniotic membrane tissue.

Preferred mesenchymal stem cells include embodiments wherein said fibroblasts are obtained from amniotic fluid.

Preferred mesenchymal stem cells include embodiments wherein said fibroblasts are obtained from bone marrow.

Preferred mesenchymal stem cells include embodiments wherein said fibroblasts are obtained from peripheral blood.

Preferred mesenchymal stem cells include embodiments wherein said fibroblasts are obtained from peripheral blood of a patient treated with G-CSF.

Preferred mesenchymal stem cells include embodiments wherein said fibroblasts are obtained from peripheral blood of a patient treated with GM-CSF.

Preferred mesenchymal stem cells include embodiments wherein said fibroblasts are obtained from peripheral blood of a patient treated with M-CSF.

Preferred mesenchymal stem cells include embodiments wherein said fibroblasts are obtained from peripheral blood of a patient treated with an agonist of CXCR4.

Preferred mesenchymal stem cells include embodiments wherein said fibroblasts are obtained from peripheral blood of a patient treated with mozibil.

Preferred mesenchymal stem cells include embodiments wherein said fibroblasts are obtained from peripheral blood of a patient treated with flt3 ligand.

Preferred mesenchymal stem cells include embodiments wherein said fibroblasts are obtained from peripheral blood of a patient treated with beta glucan.

Preferred mesenchymal stem cells include embodiments wherein said fibroblasts are obtained from peripheral blood of a patient treated with Poly IC.

Preferred mesenchymal stem cells include embodiments wherein said fibroblasts are obtained from fallopian tube tissue.

Preferred mesenchymal stem cells include embodiments wherein said fibroblasts are obtained from adipose tissue.

Preferred mesenchymal stem cells include embodiments wherein said fibroblasts are obtained from deciduous tooth tissue.

Preferred mesenchymal stem cells include embodiments wherein said fibroblasts are obtained from Wharton's Jelly.

Preferred mesenchymal stem cells include embodiments wherein said fibroblasts are obtained from omental tissue.

Preferred mesenchymal stem cells include embodiments wherein said fibroblasts are obtained from adherent pluripotent stem cells.

Preferred mesenchymal stem cells include embodiments wherein said pluripotent stem cells are induced pluripotent stem cells.

Preferred mesenchymal stem cells include embodiments wherein said pluripotent stem cells are parthenogenic derived pluripotent stem cells.

Preferred mesenchymal stem cells include embodiments wherein said pluripotent stem cells are stress derived pluripotent stem cells.

Preferred mesenchymal stem cells include embodiments wherein said pluripotent stem cells are somatic nuclear transfer derived pluripotent stem cells.

Preferred mesenchymal stem cells include embodiments wherein said fibroblasts are dedifferentiated by treatment with a “dedifferentiating agent”.

Preferred mesenchymal stem cells include embodiments wherein said dedifferentiating agent is a histone deacetylase inhibitor.

Preferred mesenchymal stem cells include embodiments wherein said histone deacetylase inhibitor is valproic acid.

Preferred mesenchymal stem cells include embodiments wherein said valproic acid is added together with lithium chloride.

Preferred mesenchymal stem cells include embodiments wherein said valproic acid is added together with a ROCK inhibitor.

Preferred mesenchymal stem cells include embodiments wherein said valproic acid is added together with GDF-15.

Preferred mesenchymal stem cells include embodiments wherein said valproic acid is added together with a cyclooxygenase 2 inhibitor.

Preferred mesenchymal stem cells include embodiments wherein said histone deacetylase inhibitor is trichostatin A.

Preferred mesenchymal stem cells include embodiments wherein said trichostatin A is added together with lithium chloride.

Preferred mesenchymal stem cells include embodiments wherein said trichostatin A is added together with a ROCK inhibitor.

Preferred mesenchymal stem cells include embodiments wherein said trichostatin A is added together with GDF-15.

Preferred mesenchymal stem cells include embodiments wherein said trichostatin A is added together with a cyclooxygenase 2 inhibitor.

Preferred mesenchymal stem cells include embodiments wherein said histone deacetylase inhibitor is phenylbutyrate.

Preferred mesenchymal stem cells include embodiments wherein said phenylbutyrate is added together with lithium chloride.

Preferred mesenchymal stem cells include embodiments wherein said phenylbutyrate is added together with a ROCK inhibitor.

Preferred mesenchymal stem cells include embodiments wherein said phenylbutyrate is added together with GDF-15.

Preferred mesenchymal stem cells include embodiments wherein said phenylbutyrate is added together with a cyclooxygenase 2 inhibitor.

Preferred mesenchymal stem cells include embodiments wherein said histone deacetylase inhibitor is vorinostat.

Preferred mesenchymal stem cells include embodiments wherein said vorinostat is added together with lithium chloride.

Preferred mesenchymal stem cells include embodiments wherein said vorinostat is added together with a ROCK inhibitor.

Preferred mesenchymal stem cells include embodiments wherein said vorinostat is added together with GDF-15.

Preferred mesenchymal stem cells include embodiments wherein said vorinostat is added together with a cyclooxygenase 2 inhibitor.

Preferred mesenchymal stem cells include embodiments wherein said mesenchymal stem cell is pretreated with monocyte conditioned media before administering for treatment of an orthopedic indication.

Preferred mesenchymal stem cells include embodiments wherein said stem cell is osmotically activated prior to administration.

Preferred mesenchymal stem cells include embodiments wherein said osmotic activation is achieved by treatment of said mesenchymal stem cell with a hypertonic or hypotonic solution for a sufficient time period to increase production of IL-10 by 25% subsequent to stimulation with beta glucan.

Preferred mesenchymal stem cells include embodiments wherein said osmotic activation is achieved by treatment of said mesenchymal stem cell with a hypertonic or hypotonic solution for a sufficient time period to increase production of IL-10 by 50% subsequent to stimulation with beta glucan.

Preferred mesenchymal stem cells include embodiments wherein said osmotic activation is achieved by treatment of said mesenchymal stem cell with a hypertonic or hypotonic solution for a sufficient time period to increase production of IL-10 by 100% subsequent to stimulation with beta glucan.

Preferred mesenchymal stem cells include embodiments wherein said monocyte is differentiated into a macrophage.

Preferred mesenchymal stem cells include embodiments wherein said macrophage possesses higher levels of COX2 as compared to monocytes.

Preferred mesenchymal stem cells include embodiments wherein said macrophage possesses higher levels of arginase as compared to monocytes.

Preferred mesenchymal stem cells include embodiments Preferred mesenchymal stem cells include embodiments wherein said macrophages are M2 macrophages.

Preferred mesenchymal stem cells include embodiments Preferred mesenchymal stem cells include embodiments wherein said macrophages are capable of producing more nitric oxide and less arginase upon activation through TLR4 as compared to control macrophages.

Preferred mesenchymal stem cells include embodiments Preferred mesenchymal stem cells include embodiments wherein said macrophages express CD16.

Preferred mesenchymal stem cells include embodiments wherein said macrophages express CD25.

Preferred mesenchymal stem cells include embodiments Preferred mesenchymal stem cells include embodiments wherein said macrophages express CCR7.

Preferred mesenchymal stem cells include embodiments wherein said macrophages express CD86

Preferred mesenchymal stem cells include embodiments Preferred mesenchymal stem cells include embodiments wherein said macrophages express CD127.

Preferred mesenchymal stem cells include embodiments Preferred mesenchymal stem cells include embodiments wherein said macrophages express interleukin-1 beta receptor.

Preferred mesenchymal stem cells include embodiments Preferred mesenchymal stem cells include embodiments wherein said macrophages express interleukin 10 receptor.

Preferred mesenchymal stem cells include embodiments Preferred mesenchymal stem cells include embodiments wherein said macrophages express TNF receptor p55.

Preferred mesenchymal stem cells include embodiments The method of claim 202, wherein said macrophages express TNF receptor p75.

Preferred mesenchymal stem cells include embodiments Preferred mesenchymal stem cells include embodiments wherein said macrophages express CD215.

Preferred mesenchymal stem cells include embodiments Preferred mesenchymal stem cells include embodiments wherein said macrophages secrete IL-1 beta.

Preferred mesenchymal stem cells include embodiments Preferred mesenchymal stem cells include embodiments wherein said macrophages secrete IL-6.

Preferred mesenchymal stem cells include embodiments Preferred mesenchymal stem cells include embodiments wherein said macrophages secrete IL-8.

Preferred mesenchymal stem cells include embodiments Preferred mesenchymal stem cells include embodiments wherein said macrophages secrete IL-12.

Preferred mesenchymal stem cells include embodiments wherein said macrophages secrete IL-15.

Preferred mesenchymal stem cells include embodiments wherein said macrophages secrete IL-17.

Preferred mesenchymal stem cells include embodiments wherein said macrophages are engineered to express HMGB-1.

Preferred mesenchymal stem cells include embodiments wherein said macrophages are engineered to express IL-12.

Preferred mesenchymal stem cells include embodiments wherein said macrophages are engineered to express IL-15.

Preferred mesenchymal stem cells include embodiments wherein said macrophages are engineered to express IL-17.

Preferred mesenchymal stem cells include embodiments wherein said macrophages are engineered to express IL-18.

Preferred mesenchymal stem cells include embodiments wherein said macrophages are engineered to express IL-23.

Preferred mesenchymal stem cells include embodiments wherein said macrophages are engineered to express IL-27.

Preferred mesenchymal stem cells include embodiments wherein said macrophages are engineered to express IL-33.

Preferred mesenchymal stem cells include embodiments wherein said macrophages are engineered to express IL-37.

Preferred embodiments include methods of treating osteoarthritis comprising intra-articular administration of a pluripotent stem cell derived mesenchymal stem cell together with a chondrocytic progenitor cell.

Preferred embodiments include methods wherein said chondrocytic progenitor cell expresses Sox9.

Preferred embodiments include methods wherein said chondrocytic progenitor cell is induced to express Sox9.

Preferred mesenchymal stem cells include embodiments wherein said chondrocytic progenitor is transfected with the Sox9 gene.

Preferred mesenchymal stem cells include embodiments wherein said transfection is performed by means of viral delivery.

Preferred embodiments include methods wherein said viral delivery is performed by use of an adenovirus.

Preferred embodiments include methods wherein said viral delivery is performed by use of an adeno-associated virus.

Preferred embodiments include methods wherein said viral delivery is performed by use of a lentivirus.

Preferred embodiments include methods wherein said viral delivery is performed by use of electroporation.

Preferred embodiments include methods wherein said chondrocytic progenitor cells are engineered to secrete aggrecan.

Preferred embodiments include methods wherein said aggrecan secretion is enhanced by exposure to TRANCE.

Preferred embodiments include methods wherein said aggrecan secretion is enhanced by exposure to TGF-beta 1.

Preferred embodiments include methods wherein said aggrecan secretion is enhanced by exposure to TGF-beta 3.

Preferred embodiments include methods wherein said chondrocytic progenitor cells are engineered to secrete cathepsin B.

Preferred embodiments include methods wherein said chondrocytic progenitor cells are engineered to secrete CHADL.

Preferred embodiments include methods wherein said chondrocytic progenitor cells are engineered to secrete chondroadherin.

Preferred embodiments include methods wherein said chondrocytic progenitor cells are engineered to secrete CRTAC1.

Preferred embodiments include methods wherein said chondrocytic progenitor cells are engineered to secrete DSPG3.

Preferred embodiments include methods wherein said chondrocytic progenitor cells are engineered to secrete sialoprotein II.

Preferred embodiments include methods wherein said chondrocytic progenitor cells are engineered to secrete CHADL.

Preferred embodiments include methods wherein said chondrocytic progenitor cells are engineered to secrete matrilin-1.

Preferred embodiments include methods wherein said chondrocytic progenitor cells are engineered to secrete matrilin-3.

Preferred embodiments include methods wherein said chondrocytic progenitor cells are engineered to secrete mattrilin-4.

Preferred embodiments include methods wherein said chondrocytic progenitor cells are engineered to secrete osteopontin.

Preferred embodiments include methods wherein said chondrocytic progenitor cells are engineered to secrete MIA.

Preferred embodiments include methods wherein said chondrocytic progenitor cells are engineered to secrete otoraplin.

Preferred embodiments include methods wherein said chondrocytic progenitor cells are engineered to secrete URB.

Preferred mesenchymal stem cells include embodiments wherein said chondrocytic progenitors are engineered by exposure to IL-6 at 10 ng/ml.

Preferred mesenchymal stem cells include embodiments wherein said chondrocytic progenitors are engineered by exposure to IL-6 at 50 ng/ml.

Preferred mesenchymal stem cells include embodiments wherein said chondrocytic progenitors are engineered by exposure to IL-6 at 100 ng/ml.

Preferred mesenchymal stem cells include embodiments wherein said chondrocytic progenitors are engineered by exposure to IL-17 at 10 ng/ml.

Preferred mesenchymal stem cells include embodiments wherein said chondrocytic progenitors are engineered by exposure to IL-17 at 20 ng/ml.

Preferred mesenchymal stem cells include embodiments wherein said chondrocytic progenitors are engineered by exposure to IL-17 at 40 ng/ml.

Preferred mesenchymal stem cells include embodiments wherein said chondrocytic progenitors are cultured an extracellular matrix substrate.

Preferred embodiments include methods wherein said extracellular matrix substrate is vitronectin.

Preferred embodiments include methods wherein said extracellular matrix substrate is fibronectin.

Preferred embodiments include methods wherein said extracellular matrix substrate is hyaluronic acid.

Preferred embodiments include methods wherein said extracellular matrix substrate is matrigel.

Preferred embodiments include methods wherein said extracellular matrix substrate is subintestinal submucosa.

Preferred embodiments include methods wherein said extracellular matrix substrate is decellularized bone matrix.

Preferred embodiments include methods wherein said extracellular matrix substrate is decellularized placental matrix.

Preferred embodiments include methods wherein said extracellular matrix substrate is decellularized adipose tissue matrix.

Preferred embodiments include methods wherein said extracellular matrix substrate is decellularized umbilical cord matrix.

Preferred embodiments include methods wherein said extracellular matrix substrate is decellularized thymic matrix.

Preferred embodiments include methods wherein said extracellular matrix substrate is decellularized ovarian tissue.

Preferred embodiments include methods wherein said extracellular matrix substrate is decellularized endometrial.

Preferred embodiments include methods wherein said extracellular matrix substrate is decellularized testicular tissue.

Preferred mesenchymal stem cells include embodiments wherein said extracellular matrix substrate is treated with one or more agents capable of acting as mitogens for said chondrocytic progenitors.

Preferred embodiments include methods wherein said chondrocytic mitogen is FGF-1.

Preferred embodiments include methods wherein said chondrocytic mitogen is FGF-2.

Preferred embodiments include methods wherein said chondrocytic mitogen is TGF-beta 1.

Preferred embodiments include methods wherein said chondrocytic mitogen is TGF-beta 3.

Preferred embodiments include methods wherein said chondrocytic mitogen is BMP2.

Preferred embodiments include methods wherein said chondrocytic mitogen is BMP4.

Preferred embodiments include methods wherein said chondrocytic mitogen is noggin.

Preferred embodiments include methods wherein said chondrocytic mitogen is IL-6.

Preferred embodiments include methods wherein said chondrocytic mitogen is IL-17A.

Preferred embodiments include methods wherein said chondrocytic mitogen is CYTL1.

Preferred embodiments include methods wherein said chondrocytic mitogen is soluble CD14.

Preferred embodiments include methods wherein said chondrocytic mitogen is thrombospondin.

Preferred embodiments include methods wherein said chondrocytic mitogen is IL-15.

Preferred embodiments include methods wherein said chondrocytic mitogen is KGF.

Preferred embodiments include methods wherein said chondrocytic mitogen is IL-22.

Preferred embodiments include methods wherein said chondrocytic mitogen is Sertoli cell conditioned media.

Preferred embodiments include methods wherein said chondrocytic mitogen is monocyte conditioned media.

Preferred embodiments include methods wherein said chondrocytic mitogen is mesenchymal stem cell conditioned media.

Preferred mesenchymal stem cells include embodiments wherein said conditioned media is obtained by mechanically stressing said cells.

Preferred embodiments include methods wherein said mechanical stress involves applying 0.375 dyn/cm2 to 2 dyn/cm2 pressure for 1-24 h/d.

Preferred embodiments include methods wherein said mechanical stress involves applying 1 dyn/cm2 to 2 dyn/cm2 pressure for 1-24 h/d.

Preferred embodiments include methods wherein said mechanical stress involves applying 1 dyn/cm2 to 2 dyn/cm2 pressure for 10-20 h/d.

Preferred embodiments include methods wherein said chondrocytic progenitor cells express micro-RNA-223-3p.

Preferred embodiments include methods wherein said chondrocytic progenitor cells express micro-RNA-21.

Preferred embodiments include methods wherein said chondrocytic progenitor cells express micro-RNA-21.

Preferred embodiments include methods wherein said chondrocytic progenitor cells express annexin A6.

Preferred embodiments include methods wherein said chondrocytic progenitor cells express CD44

Preferred embodiments include methods wherein said chondrocytic progenitor cells express CD151.

Preferred embodiments include methods wherein said chondrocytic progenitor cells express ITM-21.

Preferred embodiments include methods wherein said chondrocytic progenitor cells express micro-RNA-21.

Preferred embodiments include methods wherein said chondrocytic progenitor cells possess the ability to differentiate into hyaline cartilage.

Preferred embodiments include methods wherein said chondrocytic progenitor cells possess the ability to differentiate into fibro-cartilage.

Preferred embodiments include methods wherein said chondrocytic progenitor cells possess the ability to differentiate into elastic cartilage.

Preferred embodiments include methods wherein said pluripotent stem cell derived mesenchymal stem cells are generated by retrodifferentiation of a somatic cell.

Preferred embodiments include methods wherein said somatic cell is treated with trichostatin A.

Preferred methods include embodiments wherein said treatment with trichostatin A is performed subsequent to transfection with OCT4.

Preferred methods include embodiments wherein said transfection with OCT4 is performed when said cell is in G1/G0 state of cell cycle.

Preferred methods include embodiments wherein said transfection with OCT4 is performed simultaneously with treatment of a GSK-3 inhibitor.

Preferred methods include embodiments wherein said GSK-3 inhibitor is lithium chloride.

Preferred methods include embodiments wherein an inhibitor of histone deacetylases is administered together with or instead of trichostatin A.

Preferred methods include embodiments wherein one or more members of the Sox family of proteins is transfected into said somatic cell.

Preferred methods include embodiments wherein said Sox family member is transfected subsequent to transfection with OCT4.

Preferred methods include embodiments wherein one or more human Klf family proteins are transfected alone or together with one or more Myc family proteins are transfected alone or together with Nanog polypeptide, alone or together with the Lin 28 protein.

Preferred methods include embodiments wherein said cells treated in a manner to enhance viability prior to introduction of dedifferentiating genes.

Preferred methods include embodiments wherein said enhancement of viability is elicited by pretreatment of cells with hypoxia.

Preferred methods include embodiments wherein said cells are exposed to an atmosphere of 0.1-4% oxygen for a period of time of 5 minutes to 8 hours.

Preferred methods include embodiments wherein said cells are exposed to an atmosphere of 0.5-4% oxygen for a period of time of 5 minutes to 8 hours.

Preferred methods include embodiments wherein said cells are exposed to an atmosphere of 1-4% oxygen for a period of time of 5 minutes to 8 hours.

Preferred methods include embodiments wherein said cells are exposed to an atmosphere of 2-4% oxygen for a period of time of 5 minutes to 8 hours.

Preferred methods include embodiments wherein said cells are exposed to an atmosphere of 2-4% oxygen for a period of time of 1 to 8 hours.

Preferred methods include embodiments wherein said cells are exposed to an atmosphere of 2-4% oxygen for a period of time of 2 to 8 hours.

Preferred methods include embodiments wherein said cells are exposed to an atmosphere of 2-4% oxygen for a period of time of 4 to 8 hours.

Preferred methods include embodiments wherein said enhancement of viability is elicited by pretreatment of cells with hydrogen gas at a concentration and time period sufficient to increase levels of bcl-2 by 10% or more as compared to baseline.

Preferred methods include embodiments wherein said enhancement of viability is elicited by pretreatment of cells with hydrogen gas at a concentration and time period sufficient to increase levels of bcl-2 by 20% or more as compared to baseline.

Preferred methods include embodiments wherein said enhancement of viability is elicited by pretreatment of cells with hydrogen gas at a concentration and time period sufficient to increase levels of bcl-2 by 100% or more as compared to baseline.

Preferred methods include embodiments wherein said enhancement of viability is elicited by pretreatment of cells with hydrogen gas at a concentration and time period sufficient to increase levels of survivin by 10% or more as compared to baseline.

Preferred methods include embodiments wherein said enhancement of viability is elicited by pretreatment of cells with hydrogen gas at a concentration and time period sufficient to increase levels of survivin by 20% or more as compared to baseline.

Preferred methods include embodiments wherein said enhancement of viability is elicited by pretreatment of cells with hydrogen gas at a concentration and time period sufficient to increase levels of survivin by 100% or more as compared to baseline.

Preferred methods include embodiments wherein said enhancement of viability is elicited by pretreatment of cells with hydrogen gas at a concentration and time period sufficient to increase levels of livin by 10% or more as compared to baseline.

Preferred methods include embodiments wherein said enhancement of viability is elicited by pretreatment of cells with hydrogen gas at a concentration and time period sufficient to increase levels of livin by 20% or more as compared to baseline.

Preferred methods include embodiments wherein said enhancement of viability is elicited by pretreatment of cells with hydrogen gas at a concentration and time period sufficient to increase levels of livin by 100% or more as compared to baseline.

Preferred methods include embodiments wherein said enhancement of viability is elicited by pretreatment of cells with hydrogen gas at a concentration and time period sufficient to increase levels of IAP-1 by 10% or more as compared to baseline.

Preferred methods include embodiments wherein said enhancement of viability is elicited by pretreatment of cells with hydrogen gas at a concentration and time period sufficient to increase levels of IAP-1 by 20% or more as compared to baseline.

Preferred methods include embodiments wherein said enhancement of viability is elicited by pretreatment of cells with hydrogen gas at a concentration and time period sufficient to increase levels of IAP-1 by 100% or more as compared to baseline.

Preferred methods include embodiments wherein said enhancement of viability is elicited by pretreatment of cells with hydrogen gas at a concentration and time period sufficient to increase levels of bcl-2xL by 10% or more as compared to baseline.

Preferred methods include embodiments wherein said enhancement of viability is elicited by pretreatment of cells with hydrogen gas at a concentration and time period sufficient to increase levels of bcl-2xL by 20% or more as compared to baseline.

Preferred methods include embodiments wherein said enhancement of viability is elicited by pretreatment of cells with hydrogen gas at a concentration and time period sufficient to increase levels of bcl-2xL by 100% or more as compared to baseline.

Preferred methods include embodiments wherein said enhancement of viability is elicited by pretreatment of cells with argon gas at a concentration and time period sufficient to increase levels of bcl-2 by 10% or more as compared to baseline.

Preferred methods include embodiments wherein said enhancement of viability is elicited by pretreatment of cells with argon gas at a concentration and time period sufficient to increase levels of bcl-2 by 20% or more as compared to baseline.

Preferred methods include embodiments wherein said enhancement of viability is elicited by pretreatment of cells with argon gas at a concentration and time period sufficient to increase levels of bcl-2 by 100% or more as compared to baseline.

Preferred methods include embodiments wherein said enhancement of viability is elicited by pretreatment of cells with argon gas at a concentration and time period sufficient to increase levels of survivin by 10% or more as compared to baseline.

Preferred methods include embodiments wherein said enhancement of viability is elicited by pretreatment of cells with argon gas at a concentration and time period sufficient to increase levels of survivin by 20% or more as compared to baseline.

Preferred methods include embodiments wherein said enhancement of viability is elicited by pretreatment of cells with argon gas at a concentration and time period sufficient to increase levels of survivin by 100% or more as compared to baseline.

Preferred methods include embodiments wherein said enhancement of viability is elicited by pretreatment of cells with argon gas at a concentration and time period sufficient to increase levels of livin by 10% or more as compared to baseline.

Preferred methods include embodiments wherein said enhancement of viability is elicited by pretreatment of cells with argon gas at a concentration and time period sufficient to increase levels of livin by 20% or more as compared to baseline.

Preferred methods include embodiments wherein said enhancement of viability is elicited by pretreatment of cells with argon gas at a concentration and time period sufficient to increase levels of livin by 100% or more as compared to baseline.

Preferred methods include embodiments wherein said enhancement of viability is elicited by pretreatment of cells with argon gas at a concentration and time period sufficient to increase levels of IAP-1 by 10% or more as compared to baseline.

Preferred methods include embodiments wherein said enhancement of viability is elicited by pretreatment of cells with argon gas at a concentration and time period sufficient to increase levels of IAP-1 by 20% or more as compared to baseline.

Preferred methods include embodiments wherein said enhancement of viability is elicited by pretreatment of cells with argon gas at a concentration and time period sufficient to increase levels of IAP-1 by 100% or more as compared to baseline.

Preferred methods include embodiments wherein said enhancement of viability is elicited by pretreatment of cells with argon gas at a concentration and time period sufficient to increase levels of bcl-2xL by 10% or more as compared to baseline.

Preferred methods include embodiments wherein said enhancement of viability is elicited by pretreatment of cells with argon gas at a concentration and time period sufficient to increase levels of bcl-2xL by 20% or more as compared to baseline.

Preferred methods include embodiments wherein said enhancement of viability is elicited by pretreatment of cells with argon gas at a concentration and time period sufficient to increase levels of bcl-2xL by 100% or more as compared to baseline.

Preferred methods include embodiments wherein said enhancement of viability is elicited by pretreatment of cells with n-acetylcysteine at a concentration and time period sufficient to increase levels of bcl-2 by 10% or more as compared to baseline.

Preferred methods include embodiments wherein said enhancement of viability is elicited by pretreatment of cells with n-acetylcysteine at a concentration and time period sufficient to increase levels of bcl-2 by 20% or more as compared to baseline.

Preferred methods include embodiments wherein said enhancement of viability is elicited by pretreatment of cells with n-acetylcysteine at a concentration and time period sufficient to increase levels of bcl-2 by 100% or more as compared to baseline.

Preferred methods include embodiments wherein said enhancement of viability is elicited by pretreatment of cells with n-acetylcysteine at a concentration and time period sufficient to increase levels of survivin by 10% or more as compared to baseline.

Preferred methods include embodiments wherein said enhancement of viability is elicited by pretreatment of cells with n-acetylcysteine at a concentration and time period sufficient to increase levels of survivin by 20% or more as compared to baseline.

Preferred methods include embodiments wherein said enhancement of viability is elicited by pretreatment of cells with n-acetylcysteine at a concentration and time period sufficient to increase levels of survivin by 100% or more as compared to baseline.

Preferred methods include embodiments wherein said enhancement of viability is elicited by pretreatment of cells with n-acetylcysteine at a concentration and time period sufficient to increase levels of livin by 10% or more as compared to baseline.

Preferred methods include embodiments wherein said enhancement of viability is elicited by pretreatment of cells with n-acetylcysteine at a concentration and time period sufficient to increase levels of livin by 20% or more as compared to baseline.

Preferred methods include embodiments wherein said enhancement of viability is elicited by pretreatment of cells with n-acetylcysteine at a concentration and time period sufficient to increase levels of livin by 100% or more as compared to baseline.

Preferred methods include embodiments wherein said enhancement of viability is elicited by pretreatment of cells with n-acetylcysteine at a concentration and time period sufficient to increase levels of IAP-1 by 10% or more as compared to baseline.

Preferred methods include embodiments wherein said enhancement of viability is elicited by pretreatment of cells with n-acetylcysteine at a concentration and time period sufficient to increase levels of IAP-1 by 20% or more as compared to baseline.

Preferred methods include embodiments wherein said enhancement of viability is elicited by pretreatment of cells with n-acetylcysteine at a concentration and time period sufficient to increase levels of IAP-1 by 100% or more as compared to baseline.

Preferred methods include embodiments wherein said enhancement of viability is elicited by pretreatment of cells with n-acetylcysteine at a concentration and time period sufficient to increase levels of bcl-2xL by 10% or more as compared to baseline.

Preferred methods include embodiments wherein said enhancement of viability is elicited by pretreatment of cells with n-acetylcysteine at a concentration and time period sufficient to increase levels of bcl-2xL by 20% or more as compared to baseline.

Preferred methods include embodiments wherein said enhancement of viability is elicited by pretreatment of cells with n-acetylcysteine at a concentration and time period sufficient to increase levels of bcl-2xL by 100% or more as compared to baseline.

Preferred methods include embodiments wherein said enhancement of viability is elicited by pretreatment of cells with erythropoietin at a concentration and time period sufficient to increase levels of bcl-2 by 10% or more as compared to baseline.

Preferred methods include embodiments wherein said enhancement of viability is elicited by pretreatment of cells with erythropoietin at a concentration and time period sufficient to increase levels of bcl-2 by 20% or more as compared to baseline.

Preferred methods include embodiments wherein said enhancement of viability is elicited by pretreatment of cells with erythropoietin at a concentration and time period sufficient to increase levels of bcl-2 by 100% or more as compared to baseline.

Preferred methods include embodiments wherein said enhancement of viability is elicited by pretreatment of cells with erythropoietin at a concentration and time period sufficient to increase levels of survivin by 10% or more as compared to baseline.

Preferred methods include embodiments wherein said enhancement of viability is elicited by pretreatment of cells with erythropoietin at a concentration and time period sufficient to increase levels of survivin by 20% or more as compared to baseline.

Preferred methods include embodiments wherein said enhancement of viability is elicited by pretreatment of cells with erythropoietin at a concentration and time period sufficient to increase levels of survivin by 100% or more as compared to baseline.

Preferred methods include embodiments wherein said enhancement of viability is elicited by pretreatment of cells with erythropoietin at a concentration and time period sufficient to increase levels of livin by 10% or more as compared to baseline.

Preferred methods include embodiments wherein said enhancement of viability is elicited by pretreatment of cells with erythropoietin at a concentration and time period sufficient to increase levels of livin by 20% or more as compared to baseline.

Preferred methods include embodiments wherein said enhancement of viability is elicited by pretreatment of cells with erythropoietin at a concentration and time period sufficient to increase levels of livin by 100% or more as compared to baseline.

Preferred methods include embodiments wherein said enhancement of viability is elicited by pretreatment of cells with erythropoietin at a concentration and time period sufficient to increase levels of IAP-1 by 10% or more as compared to baseline.

Preferred methods include embodiments wherein said enhancement of viability is elicited by pretreatment of cells with erythropoietin at a concentration and time period sufficient to increase levels of IAP-1 by 20% or more as compared to baseline.

Preferred methods include embodiments wherein said enhancement of viability is elicited by pretreatment of cells with erythropoietin at a concentration and time period sufficient to increase levels of IAP-1 by 100% or more as compared to baseline.

Preferred methods include embodiments wherein said enhancement of viability is elicited by pretreatment of cells with erythropoietin at a concentration and time period sufficient to increase levels of bcl-2xL by 10% or more as compared to baseline.

Preferred methods include embodiments wherein said enhancement of viability is elicited by pretreatment of cells with erythropoietin at a concentration and time period sufficient to increase levels of bcl-2xL by 20% or more as compared to baseline.

Preferred methods include embodiments wherein said enhancement of viability is elicited by pretreatment of cells with erythropoietin at a concentration and time period sufficient to increase levels of bcl-2xL by 100% or more as compared to baseline.

Preferred methods include embodiments wherein said enhancement of viability is elicited by pretreatment of cells with FGF-1.

Preferred methods include embodiments wherein said enhancement of viability is elicited by pretreatment of cells with Z-VAD-FMK.

Preferred methods include embodiments wherein said enhancement of viability is elicited by pretreatment of cells with Z-VAD-(OME)-FMK.

Preferred methods include embodiments wherein said enhancement of viability is elicited by pretreatment of cells with Q-VD-OPH.

Preferred methods include embodiments wherein said enhancement of viability is elicited by pretreatment of cells with Belnacasan.

Preferred methods include embodiments wherein said enhancement of viability is elicited by pretreatment of cells with Z-DEVD-FMK.

Preferred methods include embodiments wherein said enhancement of viability is elicited by pretreatment of cells with Ac-DEVD-FMK.

Preferred methods include embodiments wherein said enhancement of viability is elicited by pretreatment of cells with dehydrocorydaline.

Preferred methods include embodiments wherein said enhancement of viability is elicited by pretreatment of cells with 714-X.

Preferred methods include embodiments wherein said enhancement of viability is elicited by pretreatment of cells with siRNA targeting bcl-2Xs.

Preferred methods include embodiments wherein said enhancement of viability is elicited by pretreatment of cells with antisense oligonucleotide targeting bcl-2Xs.

Preferred methods include embodiments wherein said enhancement of viability is elicited by pretreatment of cells with hammerhead ribozyme targeting bcl-2Xs.

Preferred methods include embodiments wherein said enhancement of viability is elicited by pretreatment of cells with gene edit targeting of bcl-2Xs.

Preferred methods include embodiments wherein said enhancement of viability is elicited by pretreatment of cells with siRNA targeting caspase-3.

Preferred methods include embodiments wherein said enhancement of viability is elicited by pretreatment of cells with antisense oligonucleotide targeting caspase-3.

Preferred methods include embodiments wherein said enhancement of viability is elicited by pretreatment of cells with hammerhead ribozyme targeting caspase-3.

Preferred methods include embodiments wherein said enhancement of viability is elicited by pretreatment of cells with gene edit targeting of caspase-3.

Preferred methods include embodiments wherein said enhancement of viability is elicited by pretreatment of cells with siRNA targeting caspase-.8

Preferred methods include embodiments wherein said enhancement of viability is elicited by pretreatment of cells with antisense oligonucleotide targeting caspase-8.

Preferred methods include embodiments wherein said enhancement of viability is elicited by pretreatment of cells with hammerhead ribozyme targeting caspase-8.

Preferred methods include embodiments wherein said enhancement of viability is elicited by pretreatment of cells with gene edit targeting of caspase-8.

Preferred methods include embodiments wherein said enhancement of viability is elicited by pretreatment of cells with siRNA targeting caspase-9

Preferred methods include embodiments wherein said enhancement of viability is elicited by pretreatment of cells with antisense oligonucleotide targeting caspase-9.

Preferred methods include embodiments wherein said enhancement of viability is elicited by pretreatment of cells with hammerhead ribozyme targeting caspase-9.

Preferred methods include embodiments wherein said enhancement of viability is elicited by pretreatment of cells with gene edit targeting of caspase-9.

Preferred methods include embodiments wherein said enhancement of viability is elicited by pretreatment of cells with siRNA targeting Bax.

Preferred methods include embodiments wherein said enhancement of viability is elicited by pretreatment of cells with antisense oligonucleotide targeting Bax.

Preferred methods include embodiments wherein said enhancement of viability is elicited by pretreatment of cells with hammerhead ribozyme targeting Bax.

Preferred methods include embodiments wherein said enhancement of viability is elicited by pretreatment of cells with gene edit targeting of Bax.

Preferred methods include embodiments wherein said enhancement of viability is elicited by pretreatment of cells with siRNA targeting Bid.

Preferred methods include embodiments wherein said enhancement of viability is elicited by pretreatment of cells with antisense oligonucleotide targeting Bid.

Preferred methods include embodiments wherein said enhancement of viability is elicited by pretreatment of cells with hammerhead ribozyme targeting Bid.

Preferred methods include embodiments wherein said enhancement of viability is elicited by pretreatment of cells with gene edit targeting of Bid.

Preferred methods include embodiments wherein said enhancement of viability is elicited by pretreatment of cells with siRNA targeting Hrk.

Preferred methods include embodiments wherein said enhancement of viability is elicited by pretreatment of cells with antisense oligonucleotide targeting Hrk.

Preferred methods include embodiments wherein said enhancement of viability is elicited by pretreatment of cells with hammerhead ribozyme targeting Hrk.

Preferred methods include embodiments wherein said enhancement of viability is elicited by pretreatment of cells with gene edit targeting of Hrk.

Preferred methods include embodiments wherein said enhancement of viability is elicited by pretreatment of cells with a stimulator of NF-kappa B.

Preferred methods include embodiments wherein said enhancement of viability is elicited by pretreatment of cells with minocycline.

Preferred methods include embodiments wherein said enhancement of viability is elicited by pretreatment of cells with doxycycline.

Preferred methods include embodiments wherein said enhancement of viability is elicited by pretreatment of cells with creatinine.

Preferred methods include embodiments wherein said enhancement of viability is elicited by pretreatment of cells with dicholoroacetate.

Preferred methods include embodiments wherein said enhancement of viability is elicited by pretreatment of cells with coenzyme Q10.

Preferred methods include embodiments wherein said enhancement of viability is elicited by pretreatment of cells with lipoic acid.

Preferred methods include embodiments wherein said enhancement of viability is elicited by pretreatment of cells with retinoic acid.

Preferred methods include embodiments wherein said enhancement of viability is elicited by pretreatment of cells with All Trans Retinoic Acid.

Preferred methods include embodiments wherein said enhancement of viability is elicited by pretreatment of cells with vitamin D3.

Preferred methods include embodiments wherein said enhancement of viability is elicited by pretreatment of cells with cystamine.

Preferred methods include embodiments wherein said enhancement of viability is elicited by pretreatment of cells with bucellamine.

Preferred methods include embodiments wherein said enhancement of viability is elicited by pretreatment of cells with riluozole.

Preferred methods include embodiments wherein said enhancement of viability is elicited by pretreatment of cells with linderalactone.

Preferred embodiments include methods wherein said orthopedic condition is degenerative arthritis.

Preferred embodiments include methods wherein said orthopedic condition is rheumatoid arthritis.

Preferred embodiments include methods wherein said orthopedic condition is plantar fascitis.

Preferred embodiments include methods wherein said orthopedic condition is humerus epicondylitis.

Preferred embodiments include methods wherein said orthopedic condition is humerus myositis ossificans.

Preferred embodiments include methods wherein said rheumatoid arthritis is associated with formation of a pannus.

Preferred embodiments include methods wherein said pannus is angiogenesis dependent.

Preferred embodiments include methods wherein said pannus is associated with elevated production of joint TNF-alpha as compared to an age-matched healthy joint.

Preferred embodiments include methods wherein said pannus is associated with elevated production of joint RANK ligand as compared to an age-matched healthy joint.

Preferred embodiments include methods wherein said pannus is associated with elevated production of joint interferon gamma as compared to an age-matched healthy joint.

Preferred embodiments include methods wherein said pannus is associated with elevated production of joint MMP1 as compared to an age-matched healthy joint.

Preferred embodiments include methods wherein said pannus is associated with elevated production of joint MMP3 as compared to an age-matched healthy joint.

Preferred embodiments include methods wherein said pannus is associated with elevated production of joint MMP5 as compared to an age-matched healthy joint.

Preferred embodiments include methods wherein said pannus is associated with elevated production of joint MMP9 as compared to an age-matched healthy joint.

Preferred embodiments include methods wherein said pannus is associated with elevated production of joint neutrophil elastase as compared to an age-matched healthy joint.

Preferred embodiments include methods wherein said pannus is associated with elevated production of joint IL-8 as compared to an age-matched healthy joint.

Preferred embodiments include methods wherein said pannus is associated with elevated production of joint IL-12 as compared to an age-matched healthy joint.

Preferred embodiments include methods wherein said pannus is associated with elevated production of joint IL-15 as compared to an age-matched healthy joint.

Preferred embodiments include methods wherein said pannus is associated with elevated production of joint IL-17 as compared to an age-matched healthy joint.

Preferred embodiments include methods wherein said pannus is associated with elevated production of joint IL-17c as compared to an age-matched healthy joint.

Preferred embodiments include methods wherein said pannus is associated with elevated production of joint IL-18 as compared to an age-matched healthy joint.

Preferred embodiments include methods wherein said pannus is associated with elevated production of joint IL-23 as compared to an age-matched healthy joint.

Preferred embodiments include methods wherein said pannus is associated with elevated production of joint IL-27 as compared to an age-matched healthy joint.

Preferred embodiments include methods wherein said pannus is associated with elevated production of joint IL-33 as compared to an age-matched healthy joint.

Preferred embodiments include methods wherein said pannus is associated with elevated production of joint low molecular weight hyaluronic acid fragments as compared to an age-matched healthy joint.

Preferred embodiments include methods wherein said pannus is associated with elevated production of joint MMP1 as compared to an age-matched healthy joint.

Preferred embodiments include methods wherein said rheumatoid arthritis is treated with said cell therapy of claim 1, and further treated with one or more agents capable of suppressing the underlying autoimmune cause of said rheumatoid arthritis.

Preferred methods include embodiments wherein said agent capable of suppressing the underlying autoimmune cause of said rheumatoid arthritis is a peptide pulsed tolerance promoting cell.

Preferred methods include embodiments wherein said peptide comprises one or more peptides associated with articular damage.

Preferred methods include embodiments wherein said peptide is a derivative of collagen.

Preferred embodiments include methods wherein said peptide is collagen II.

Preferred embodiments include methods wherein said peptide is collagen IV.

Preferred embodiments include methods wherein said peptide is collagen V.

Preferred embodiments include methods wherein said peptide is collagen VII.

Preferred embodiments include methods wherein said peptide is citrullinated collagen II.

Preferred embodiments include methods wherein said peptide is citrullinated IV.

Preferred embodiments include methods wherein said peptide is citrullinated V.

Preferred embodiments include methods wherein said peptide is citrullinated VII.

Preferred embodiments include methods wherein said peptide is chondrocyte glycoprotein 39.

Preferred embodiments include methods wherein said peptide is hsp35.

Preferred embodiments include methods wherein said peptide is hsp75.

Preferred embodiments include methods wherein said peptide is calreticulin.

Preferred methods include embodiments wherein said tolerance promoting cell is a myeloid suppressor cell.

Preferred embodiments include methods wherein said myeloid suppressor cell is generated by stimulation of bone marrow in vivo with IL-4 and GM-CSF.

Preferred embodiments include methods wherein said myeloid suppressor cell is generated by stimulation of bone marrow in vivo with IL-4 and G-CSF.

Preferred embodiments include methods wherein said myeloid suppressor cell is generated by stimulation of bone marrow in vivo with GM-CSF and VEGF.

Preferred embodiments include methods wherein said myeloid suppressor cell is generated by stimulation of bone marrow in vivo with IL-10 and flt-3 ligand.

Preferred embodiments include methods wherein said myeloid suppressor cell is generated by culture of umbilical cord blood CD34 cells in IL-10 and GM-CSF in the presence of fibroblast feeder layers.

Preferred embodiments include methods wherein said myeloid suppressor cell is generated by culture of umbilical cord blood CD34 cells in IL-10 and GM-CSF in the presence of mesenchymal stem cell feeder layers.

Preferred methods include embodiments wherein said mesenchymal stem cells express CD105.

Preferred methods include embodiments wherein said mesenchymal stem cells express PD-L1.

Preferred methods include embodiments wherein said mesenchymal stem cells are transfected to express TGF-beta.

Preferred methods include embodiments wherein said mesenchymal stem cells express c-kit.

Preferred methods include embodiments wherein said mesenchymal stem cells express CD73.

Preferred methods include embodiments wherein said mesenchymal stem cells express TrkA.

Preferred methods include embodiments wherein said mesenchymal stem cells express complement component 3 receptor.

Preferred methods include embodiments wherein said mesenchymal stem cells express complement component 5 receptor.

Preferred methods include embodiments wherein said mesenchymal stem cells express c-met.

Preferred methods include embodiments wherein said mesenchymal stem cells express c-maf.

Preferred methods include embodiments wherein said tolerance promoting cell is a B cell.

Preferred methods include embodiments wherein said B cell is a type 1 B cell.

Preferred methods include embodiments wherein said type 1 B cell secretes 50% more interleukin-10 after stimulation with pokeweed mitogen as compared to a conventional B cell.

Preferred methods include embodiments wherein said type 1 B cell secretes 100% more interleukin-10 after stimulation with pokeweed mitogen as compared to a conventional B cell.

Preferred methods include embodiments wherein said type 1 B cell secretes 100% more interleukin-10 after stimulation with pokeweed mitogen as compared to a conventional B cell.

Preferred methods include embodiments wherein said type 1 B cell secretes 50% more interleukin-35 after stimulation with pokeweed mitogen as compared to a conventional B cell.

Preferred methods include embodiments wherein said type 1 B cell secretes 100% more interleukin-35 after stimulation with pokeweed mitogen as compared to a conventional B cell.

Preferred methods include embodiments wherein said type 1 B cell secretes 100% more interleukin-35 after stimulation with pokeweed mitogen as compared to a conventional B cell.

Preferred methods include embodiments wherein said type 1 B cell possesses more PD-L1 per surface area as compared to a conventional B cell.

Preferred methods include embodiments wherein said type 1 B cell possesses more BTLA4 per surface area as compared to a conventional B cell.

Preferred methods include embodiments wherein said type 1 B cell possesses more HLA-G per surface area as compared to a conventional B cell.

Preferred methods include embodiments wherein said type 1 B cell possesses more LAG3 per surface area as compared to a conventional B cell.

Preferred methods include embodiments wherein said type 1 B cell possesses more TIM3 per surface area as compared to a conventional B cell.

Preferred methods include embodiments wherein said type 1 B cell possesses more IL-10 receptor per surface area as compared to a conventional B cell.

Preferred methods include embodiments wherein said type 1 B cell possesses more TGF-beta per surface area as compared to a conventional B cell.

Preferred methods include embodiments wherein said type 1 B cell is capable of inducing convention of naïve T cells to T regulatory cells.

Preferred methods include embodiments wherein said T regulatory cells express FoxP3.

Preferred methods include embodiments wherein said T regulatory cells express GITR ligand.

Preferred methods include embodiments wherein said T regulatory cells express LAP.

Preferred methods include embodiments wherein said T regulatory cells express granzyme B.

Preferred methods include embodiments wherein said T regulatory cells express perforin.

Preferred methods include embodiments wherein said T regulatory cells are capable of suppressing production of interferon gamma from an activated T cell.

Preferred methods include embodiments wherein said T regulatory cells are capable of suppressing production of IL-2 from an activated T cell.

Preferred methods include embodiments wherein said T regulatory cells are capable of suppressing production of IL-12 from an activated T cell.

Preferred methods include embodiments wherein said T regulatory cells are capable of suppressing production of IL-15 from an activated T cell.

Preferred methods include embodiments wherein said T regulatory cells are capable of suppressing production of IL-17 from an activated T cell.

Preferred methods include embodiments wherein said T regulatory cells are capable of suppressing production of IL-18 from an activated T cell.

Preferred methods include embodiments wherein said T regulatory cells are capable of suppressing production of IL-21 from an activated T cell.

Preferred methods include embodiments wherein said T regulatory cells are capable of suppressing production of IL-23 from an activated T cell.

Preferred methods include embodiments wherein said T regulatory cells are capable of suppressing production of IL-27 from an activated T cell.

Preferred methods include embodiments wherein said T regulatory cells are capable of stimulating production of IL-10 from an activated T cell.

Preferred methods include embodiments wherein said T regulatory cells are capable of stimulating production of TGF-beta from an activated T cell.

Preferred methods include embodiments wherein said T regulatory cells are capable of stimulating production of HLA-G from an activated T cell.

Preferred methods include embodiments wherein said T regulatory cells are capable of stimulating production of IL-1 receptor antagonist from an activated T cell.

Preferred methods include embodiments wherein said T regulatory cells are capable of stimulating production of IL-12 p40 homodimers from an activated T cell.

Preferred methods include embodiments wherein said T regulatory cells are capable of stimulating production of IL-10 from an activated T cell.

Preferred methods include embodiments wherein said T regulatory cells are capable of stimulating production of CTLA4 from an activated T cell.

Preferred methods include embodiments wherein said T regulatory cells are capable of stimulating production of PD-L1 from an activated T cell.

Preferred methods include embodiments wherein said T regulatory cells are capable of suppressing proliferation of an activated T cell.

Preferred methods include embodiments wherein proliferation of said activated T cell is induced by crosslinking of the T cell receptor.

Preferred methods include embodiments wherein proliferation of said activated T cell is induced by activation of the IL-2 receptor.

Preferred methods include embodiments wherein proliferation of said activated T cell is induced by activation of the IL-6 receptor.

Preferred methods include embodiments wherein proliferation of said activated T cell is induced by activation of the IL-7 receptor.

Preferred methods include embodiments wherein proliferation of said activated T cell is induced by activation of the IL-15 receptor.

Preferred methods include embodiments wherein proliferation of said activated T cell is induced by treatment with a lectin.

Preferred methods include embodiments wherein said lectin is cyanovirin.

Preferred methods include embodiments wherein said lectin is concanavalin A.

Preferred methods include embodiments wherein said lectin is mannosyl-α1,6-mannose.

Preferred methods include embodiments wherein said lectin is mannosyl-α1,2-mannose.

Preferred methods include embodiments wherein said lectin is Phaseolus vulgaris erythroagglutinin.

Preferred methods include embodiments wherein said lectin is soybean agglutinin.

Preferred methods include embodiments wherein said lectin is arcelin-1.

Preferred methods include embodiments wherein said lectin is hen ovalbumin.

Preferred methods include embodiments wherein said lectin is orosomucoid.

Preferred methods include embodiments wherein said lectin is ovomucoid.

Preferred methods include embodiments wherein said lectin is bovine lactotransferrin.

Preferred methods include embodiments wherein said lectin is bovine human serotransferrin.

Preferred methods include embodiments wherein said tolerance promoting cell is an immature dendritic cell.

Preferred methods include embodiments wherein said immature dendritic cell is myeloid derived.

Preferred methods include embodiments wherein said immature dendritic cell is lymphoid derived.

Preferred methods include embodiments wherein said immature dendritic cell possesses less costimulatory ability as compared to a mature dendritic cell.

Preferred methods include embodiments wherein said costimulatory activity is ability to stimulate T cell cytokine production.

Preferred methods include embodiments wherein said costimulatory activity is ability to stimulate T cell proliferative activity.

Preferred methods include embodiments wherein said costimulatory activity is ability to stimulate T cell mediated macrophage activation.

Preferred methods include embodiments wherein said costimulatory activity is ability to stimulate T cell mediated isotype switching.

Preferred methods include embodiments wherein said costimulatory activity is ability to stimulate T cell mediated bone resorption.

Preferred methods include embodiments wherein said costimulatory activity is ability to stimulate T cell mediated cytotoxicity.

Preferred methods include embodiments wherein said costimulatory activity is expression of CD5.

Preferred methods include embodiments wherein said costimulatory activity is expression of ICAM-1.

Preferred methods include embodiments wherein said costimulatory activity is expression of LFA-3.

Preferred methods include embodiments wherein said costimulatory activity is expression of CD40.

Preferred methods include embodiments wherein said costimulatory activity is expression of CD80.

Preferred methods include embodiments wherein said costimulatory activity is expression of CD86.

Preferred methods include embodiments wherein said costimulatory activity is secretion of IL-2.

Preferred methods include embodiments wherein said costimulatory activity is secretion of IL-5.

Preferred methods include embodiments wherein said costimulatory activity is secretion of IL-7.

Preferred methods include embodiments wherein said costimulatory activity is secretion of IL-15.

Preferred methods include embodiments wherein said costimulatory activity is secretion of IL-18.

Preferred embodiments include methods of treating degenerative disc disease comprising the steps of: a) Obtaining a pluripotent stem cell; b) Pretreating the nucleus pulposus of the patient in need treatment with an intermediary cell population; c) Activating said intermediary cell population; and d) administering said pluripotent stem cell into said nucleus pulposus.

### DETAILED DESCRIPTION OF THE INVENTION

The invention provides means of generating cellular therapeutics for treatment of orthopedic disorders. In one embodiment generation of cartilage and/or various articular tissues is disclosed through differentiation of pluripotent stem cells into mesenchymal stem and subsequently inducing said mesenchymal stem cells to differentiate into chondrocytes and/or chondrogenic cells. Various types of chondrogenic cells can be generated which possess activity in regeneration of joint tissue. Treatments of osteoarthritis, non-union bone fractions, disc degenerative disease and other orthopedic indications are provided. Chondrogenesis, or the process of forming cartilage, involves the commitment of pluripotential mesenchymal cells to the chondrocyte lineage and their differentiation along this pathway. This process requires the cells to produce and respond to a number of cytokines and growth factors, including platelet derived growth factor (PDGF), insulin like growth factor (IGF), basic fibroblast growth factor (bFGF), transforming growth factor beta (TGFβ), bone morphogenetic protein (BMP), and cartilage derived growth factor (CDGF). Once the cells enter the chondrocyte lineage, they produce and maintain a matrix rich in collagen type II and proteoglycan aggregate. Chondrocytes that are found in articular cartilage synthesize and maintain this matrix over long periods of time. At the base of the articular cartilage, where it interfaces with subchondral bone, there is a small region of cells that continue to differentiate forming calcified cartilage. This region is called the tidemark and is similar in many ways to the growth plate cartilage of long bones.

By “Articular cartilage” is hyaline cartilage and is 2 to 4 mm thick. It is composed of a dense extracellular matrix (ECM) with a sparse distribution of chondrocytes The ECM is principally composed of water, collagen, and proteoglycans, with other non-collagenous proteins and glycoproteins present in lesser amounts. Along with collagen fiber ultrastructure and ECM, chondrocytes contribute to the various zones of articular cartilage—the superficial zone, the middle zone, the deep zone, and the calcified zone. Within each zone, 3 regions can be identified—the pericellular region, the territorial region, and the interterritorial region. The thin superficial (tangential) zone protects deeper layers from shear stresses and makes up approximately 10% to 20% of articular cartilage thickness. The collagen fibers of this zone (primarily, type II and IX collagen) are packed tightly and aligned parallel to the articular surface. The superficial layer contains a relatively high number of flattened chondrocytes, and the integrity of this layer is imperative in the protection and maintenance of deeper layers. This zone is in contact with synovial fluid and is responsible for most of the tensile properties of cartilage, which enable it to resist the sheer, tensile, and compressive forces imposed by articulation. Immediately deep to the superficial zone is the middle (transitional) zone, which provides an anatomic and functional bridge between the superficial and deep zones. The middle zone represents 40% to 60% of the total cartilage volume, and it contains proteoglycans and thicker collagen fibrils. In this layer, the collagen is organized obliquely, and the chondrocytes are spherical and at low density. Functionally, the middle zone is the first line of resistance to compressive forces. The deep zone is responsible for providing the greatest resistance to compressive forces, given that collagen fibrils are arranged perpendicular to the articular surface. The deep zone contains the largest diameter collagen fibrils in a radial disposition, the highest proteoglycan content, and the lowest water concentration. The chondrocytes are typically arranged in columnar orientation, parallel to the collagen fibers and perpendicular to the joint line. The deep zone represents approximately 30% of articular cartilage volume. The tide mark distinguishes the deep zone from the calcified cartilage. The deep zone is responsible for providing the greatest amount of resistance to compressive forces, given the high proteoglycan content. Of note, the collagen fibrils are arranged perpendicular to the articular cartilage. The calcified layer plays an integral role in securing the cartilage to bone, by anchoring the collagen fibrils of the deep zone to subchondral bone. In this zone, the cell population is scarce and chondrocytes are hypertrophic.

By “Biocompatible material” refers to any organic or inorganic compound that can be safely and effectively introduced into a patient's body for tissue engineering purposes. These include, but are not limited to: 1) materials with organic, viscous and gelling properties, such as, but not limited to, alginate, collagen, fibrin, and hyaline. “Materials with organic and malleable properties” refers to materials that can be used to create a solid scaffold, including, but limited to, polyglycolic polylactic acid (PGLA) sutures (Vicryl™) or other woven suture-like material; solid materials of inorganic (metal, plastic or other biocompatible solid) or organic (bone allografts) properties suitable for insertion through a cartilage defect into the underlying cancellous bone to provide an anchor for sutures during an orthopedic procedure. The biocompatible material also includes a matrix comprising an isolated adipose tissue-derived stem cell differentiated to express at least one characteristic of an osteoblast.

By “BMP-2” refers to the family of bone morphogenetic proteins of the type 2, derived from any species, and may include mimetics and variants thereof. Reference to BMP-2 herein is understood to be a reference to any one of the currently identified forms, including BMP-2A and BMP-2B, as well as to BMP-2 species identified in the future. The term “BMP-2” also includes polypeptides derived from the sequence of any known BMP-2 whose mature sequence is at least about 75% homologous with the sequence of a mature human BMP-, which reference sequence may be found in Genbank, accession number NP_001191. BMP-2 signals via two types of receptors (BRI and BRII) that are expressed at the cell surface as homomeric as well as heteromeric complexes. Prior to ligand binding, a low but measurable level of BMP-receptors is found in preformed hetero-oligomeric complexes. The major fraction of the receptors is recruited into hetero-oligomeric complexes only after ligand addition. For this, BMP-2 binds first to the high affinity receptor BRI and then recruits BRII into the signaling complex. However, ligand binding to the preformed complex composed of BRII and BRI is still required for signaling, suggesting that it may mediate activating conformational changes. Signals induced by binding of BMP-2 to preformed receptor complexes activate the Smad pathway, whereas BMP-2-induced recruitment of receptors activates a different, Smad-independent pathway resulting in the induction of alkaline phosphatase activity via p38 MAPK. “BMP2 agents” include molecules that function similarly to BMP2 by binding and activating its receptors as described above. Molecules useful as BMP2 agents include derivatives, variants, and biologically active fragments of naturally occurring BMP2. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein. The variant polypeptides can have post-translational modifications not found on the natural BMP2 protein. Fragments and fusion proteins of soluble BMP2, particularly biologically active fragments and/or fragments corresponding to functional domains, are of interest. Fragments of interest will typically be at least about 10 aa to at least about 15 aa in length, usually at least about 50 aa in length, but will usually not exceed about 142 aa in length, where the fragment will have a stretch of amino acids that is identical to BMP2. A fragment “at least 20 aa in length,” for example, is intended to include 20 or more contiguous amino acids from, for example, the polypeptide encoded by a cDNA for BMP2. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods. In some embodiments, a dose of BMP2 is provided in an implant, e.g. a matrix or scaffold for localized delivery of the factor, where the BMP2 is provided as a BMP2 protein or active fragment thereof. The effective dose may be determined based on the specific tissue, rate of release from the implant, size of the implant, and the like. and may be empirically determined by one of skill in the art. The dose may provide for biological activity equivalent to 1 μg BMP2 protein, 10 μg, 100 μg, 1 mg, 5 mg, 10 mg, 25 mg, 50 mg, 75 mg, 100 mg, 250 mg, 500 mg, 750 mg, 1 g of BMP2 protein. The dose may be administered at a single time point, e.g. as a single implant; or may be fractionated, e.g. delivered in a microneedle configuration. The dose may be administered, once, two, three time, 4 times, 5 times, 10 times, or mare as required to achieve the desired effect, and administration may be daily, every 2 days, every 3 days, every 4 days, weekly, bi-weekly, monthly, or more.

By “Chondrocytes” or “cartilage cells” refer to cells that are capable of expressing a characteristic biochemical marker of chondrocytes, including but not limited to collagen type II, chondroitin sulfate, keratin sulfate and characteristic morphologic markers, including but not limited to the rounded morphology observed in culture, and able to secrete collagen type II.

By “Chondroinductive agent”, “chondroinductive factor” or “chondroinductive substance” refer to any natural or synthetic, organic or inorganic chemical or biochemical compound, factor or combination of compounds or factors, or any mechanical or physical device, container, influence or force that can be applied to human adipose tissue-derived stromal cells so as to effect in vitro chondrogenic induction or the production of chondrocytes. The chondroinductive agent is selected individually or in combination from the groups consisting of i) a glucocorticoid such as dexamethasone; ii) a member of the transforming growth factor-beta (TGF-β) superfamily such as bone morphogenic protein (BMP: BMP-2,-4; TGF-β1,2,3; insulin-like growth factor (IGF); platelet derived growth factor (PDGF); epidermal growth factor (EGF); acidic fiborblastic growth factor; basic fibroblastic growth factor, hepatocytic growth factor, keratocytic growth factor, osteogenic proteins (OP-1,2,3); inhibin A or chondrogenic stimulating activity factor; iii) a component of the collagenous extracellular matrix such as collagen I; iv) a vitamin A analogue such as retinoic acid; and v) ascorbate or other vitamin C-related analogue.

By “Non-peptide growth factors” refers to steroids, retinoids and other chemical compounds or agents that induce differentiation. These include, but are not limited to, 1,25 dihydroxyvitamin D3, dexamethasone, hydrocortisone, retinoic acid, and 9-cis retinoic acid.

By “Developmental phenotype” is the potential of a cell to acquire a particular physical phenotype through the process of differentiation.

By “Genotype” is the expression at least one messenger RNA transcript of a gene associated with a differentiation pathway.

By “Autoimmune disease” is intended to encompass any immune mediated process, humoral or cellular, that results in the rejection and destruction of the hosts' end organ. The etiology of this process can include, but is not limited to, an immune response to an infection by an agent such as a virus, an inborn metabolic propensity to autoimmune dysfunction, or a chemical exposure.

By “biomaterial matrices” is meant any biocompatible compound, resorbable or non-resorbable, which is able support the adherence, growth, differentiation, proliferation, vascularization, and three-dimensional modeling of adipose tissue-derived stem cells into a soft tissue or adipose tissue depot either in vivo or ex vivo. These include, but are not limited to, polylactic acid, poly-glycolic acid, hyaluronates, derivatives of glycosaminoglycans, alginate, collagen type I and its derivatives, collagen type IV and its derivatives, any other collagen type and its derivatives, or any combination thereof.

By “chemical inducing factors” is meant any chemical agent, either protein, lipid, or carbohydrate in character, which enhances the adherence, growth, differentiation, proliferation, vascularization and three-dimensional modeling of adipose tissue-derived stem or stromal cells into articular cartilage depot either in vivo or ex vivo. These include, but are not limited to, monobutyrin, thiazolidinediones, glucocorticoids, and long chain fatty acids.

By “protein growth factors and cytokines” is meant any protein hormone, growth factor, or cytokine which enhances the adherence, growth, differentiation, proliferation, vascularization, and three-dimensional modeling of adipose tissue-derived stem cells into articular cartilage depot either in vivo or ex vivo. These include but are not limited to, vascular endothelial growth factor, fibroblast growth factor (basic), bone morphogenetic protein 4, bone morphogenetic protein 7, insulin and its analogues, leptin, and growth hormone.

The in one embodiment the invention teaches the generation of pluripotent stem cells for use in creation of either chondrogenic progenitors or mesenchymal stem cells capable of generating said chondrogenic progenitors. In order to induce the generation of pluripotent stem cells, the utilization of enhanced methodology of dedifferentiation protocols is disclosed. In one embodiment, proteins and derivative proteins are used to induce the formation of pluripotent stem cell can include any combination of AIRE polypeptides, PIM-1 polypeptides, Oct3/4 polypeptides, Sox family polypeptides (e.g., Sox2 polypeptides), Klf family of polypeptides (e.g., Klf4 polypeptides), Myc family polypeptides (e.g., c-Myc), Nanog polypeptides, and Lin28 polypeptides. These polypeptides are administered, in one embodiment of the invention, to cells which already possess a dedifferentiated phenotype. In some embodiments stem cells are utilized to generated pluripotent stem cells. Said stem cells may be mesenchymal stem cells, or in some embodiments hematopoietic stem cells.

Cells to be dedifferentiated are subsequently made to express proteins/polypeptides associated with dedifferentiation. This can be accomplished through the administration of nucleic acid vectors designed to express AIRE, Pim-1, Oct3/4, Sox2, Klf4, and c-Myc.

In some embodiments AIRE, Pim-1, Oct3/4, Sox2, Klf4, and c-Myc polypeptides can be directly delivered into target cells to obtain induced pluripotent stem cells using a polypeptide transfection method (e.g., liposome or electroporation). In one embodiment, nucleic acid vectors designed to express Oct3/4, Sox2, and Klf4 polypeptides, and not a c-Myc polypeptide, can be used to obtain induced pluripotent stem cells. In some cases, Oct3/4, Sox2, and Klf4 polypeptides can be directly delivered into target cells to obtain induced pluripotent stem cells using a polypeptide transfection method. Any appropriate cell type can be used to obtain induced pluripotent stem cells. For example, skin, lung, heart, liver, blood, kidney, or muscle cells can be used to obtain induced pluripotent stem cells. Such cells can be obtained from any type of mammal including, without limitation, humans, mice, rats, dogs, cats, cows, pigs, or monkeys. In addition, any stage of the mammal can be used, including mammals at the embryo, neonate, newborn, or adult stage. For example, fibroblasts obtained from an adult human patient can be used to obtain induced pluripotent stem cells. Such induced pluripotent stem cells can be used to treat that same human patient (or to treat a different human) or can be used to create differentiated cells that can be used to treat that same human patient (or a different human). For example, somatic cells from a human patient can be treated as described herein to obtain induced pluripotent stem cells. The obtained induced pluripotent stem cells can be differentiated into cardiomyocytes that can be implanted into that same human patient. In some cases, the obtained induced pluripotent stem cells can be directly administered to that same human patient. Any appropriate method can be used to introduce nucleic acid (e.g., nucleic acid encoding polypeptides designed to induce pluripotent stem cells from cells) into a cell. For example, nucleic acid encoding polypeptides (e.g., Oct3/4, Sox2, Klf4, and c-Myc polypeptides) designed to induce pluripotent stem cells from other cells (e.g., non-embryonic stem cells) can be transferred to the cells using recombinant viruses that can infect cells, or liposomes or other non-viral methods such as electroporation, microinjection, transposons, phage integrases, or calcium phosphate precipitation, that are capable of delivering nucleic acids to cells. The exogenous nucleic acid that is delivered typically is part of a vector in which a regulatory element such as a promoter is operably linked to the nucleic acid of interest. The promoter can be constitutive or inducible. Non-limiting examples of constitutive promoters include cytomegalovirus (CMV) promoter and the Rous sarcoma virus promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus. Additional regulatory elements that may be useful in vectors, include, but are not limited to, polyadenylation sequences, translation control sequences (e.g., an internal ribosome entry segment, IRES), enhancers, or introns. Such elements may not be necessary, although they can increase expression by affecting transcription, stability of the mRNA, translational efficiency, or the like. Such elements can be included in a nucleic acid construct as desired to obtain optimal expression of the nucleic acids in the cells. Sufficient expression, however, can sometimes be obtained without such additional elements. Vectors also can include other elements. For example, a vector can include a nucleic acid that encodes a signal peptide such that the encoded polypeptide is directed to a particular cellular location (e.g., the cell surface) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

The invention provides numerous means of achieving dedifferentiation for the generation of pluripotent stem cells. In some embodiments, viral vectors can be used to introduce sternness-related factors, such as Oct3/4, Klf4, Sox2 and c-Myc. Examples of viral vectors include, without limitation, vectors based on DNA or RNA viruses, such as adenovirus, adeno-associated virus (AAV), retroviruses, lentiviruses, vaccinia virus, measles viruses, herpes viruses, baculoviruses, and papilloma virus vectors. See, Kay et al., Proc. Natl. Acad. Sci. USA, 94:12744-12746 (1997) for a review of viral and non-viral vectors. Viral vectors can be modified so the native tropism and pathogenicity of the virus has been altered or removed. The genome of a virus also can be modified to increase its infectivity and to accommodate packaging of the nucleic acid encoding the polypeptide of interest. In some cases, the induced pluripotent stem cells provided herein can be obtained using viral vectors that do not integrate into the genome of the cells. Such viral vectors include, without limitation, adenoviral vectors, AAV vectors, baculovirus vectors, and herpesvirus vectors. For example, cells obtained from a human can be provided nucleic acid encoding human Oct3/4, Sox2, Klf4, and c-Myc polypeptides using viral vectors that do not integrate the exogenous nucleic acid into the cells. Once the polypeptides are expressed and induced pluripotent stem cells are obtained, the induced pluripotent stem cells can be maintained in culture such that the induced pluripotent stem cells are devoid of the exogenous nucleic acid. Any appropriate non-viral vectors can be used to introduce stemness-related factors, such as Oct3/4, Klf4, Sox2, and c-Myc. Examples of non-viral vectors include, without limitation, vectors based on plasmid DNA or RNA, retroelement, transposon, and episomal vectors. Non-viral vectors can be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. See, Felgner et al., J. Biol. Chem., 269:2550-2561 (1994). High efficiency liposomes are commercially available. See, for example, SuperFect® from Qiagen (Valencia, Calif.). In some cases, induced pluripotent stem cells can be obtained using culture conditions that do not involve the use of serum or feeder cells. For example, cells obtained from a human can be provided nucleic acid encoding human Oct3/4, Sox2, Klf4, and c-Myc polypeptides and cultured using media lacking serum (e.g., human or non-human serum) and lacking feeder cells (e.g., human or non-human feeder cells).

In one embodiment, of the invention generation of chondrogenic progenitors is performed by treatment of pluripotent derived mesenchymal progenitor cells with various agents useful for stimulation of chondrocytic, or when needed osteogenic differentiation. For generation of cartilage, numerous protocols may be utilized. First, mesenchymal stem cells are prepared in order to accomplish a chondrogenic differentiation from the mesenchymal stem cells. The mesenchymal stem cells are preferably human, and may be derived from pluripotent stem cells, in some embodiments through culture on BMP2 containing extracellular matrices. The differentiation process of mesenchymal stem cell to chondrocyte can be performed on a monolayer culture (Two-dimensional culture) of the prepared mesenchymal stem cells is conducted, for example, in a culture dish. The monolayer culture is performed by placing the mesenchymal stem cells into a growth medium supplemented with bovine fetal serum and antibiotics in a 150 mm culture dish at a concentration of about 1×106 cells. The culture may be incubated in a 5% CO2 incubator at 37° C. by attaching the cells to the culture dish. The medium may be exchanged once every three days and a subculture may be performed once every week. The number of mesenchymal stem cells suitable for a three-dimensional culture may be secured through these monolayer cultures. After the monolayer culture is completed, the mesenchymal stem cells are detached from the culture dish to conduct a three-dimensional culture. The three-dimensional culture may include a pellet culture conducted in pellet form or a culture using alginate beads or PGA (acronym for Polyglycolide and an aliphatic polyester which is a kind of poly(a-hydroxy acid)) scaffold. The three-dimensional forms are manufactured, placed into the chondrogenic differentiation medium, and then put in the incubator. The chondrogenic differentiation medium may be exchanged, for example, once every three days. The three-dimensional structure of the mesenchymal stem cells in the chondrogenic differentiation medium is withdrawn from the incubator, placed into a centrifuge illustrated in FIG. 1, and then rotated preferably at about 10 to about 200 G-force to apply centrifugal force to the mesenchymal stem cells. These processes may be performed for about 10 to about 30 minutes every day for 2 to 4 consecutive weeks. Through these processes, cells having characteristics of chondrogenic cells or chondrocytes may be differentiated from mesenchymal stem cells. Furthermore, chondrocytes may be differentiated. Whether chondrogenic cells or chondrocytes are differentiated may be determined by using safranin-O and/or an immunohistological analysis to identify whether GAG protein and type II collagen protein have color development, and whether lacunas (characteristics of chondrocytes) are formed. Whether chondrogenic cells or chondrocytes are differentiated may be also determined by identifying the expressions of type II collagen (a marker for chondrogenic differentiation) and aggrecan gene. Chondrogenic cells or chondrocytes differentiated by these methods may be used as a composition for treating diseases caused by cartilage damage. The PGA or alginate has the property of being degraded and absorbed spontaneously in vivo. Thus, chondrogenic cells or chondrocytes differentiated by using the PGA scaffolds or alginates may be used as a composition in which the PGA or alginates are included. For example, mesenchymal stem cells, which are derived from a born marrow derived pluripotent stem cells of a patient who has cartilage injuries, can be cultured and applied by a centrifugal force to be differentiated to chondrogenic cells or chondrocytes. These chondrogenic cells or chondrocytes can be injected into the cartilage of the patient as a composition for medical cure. Furthermore, an artificial joint may be manufactured by differentiating mesenchymal stem cells of the present invention into chondrogenic cells or chondrocytes on a joint-shaped three-dimensional support matrix. For the generation of chondrogenic cells, it may be desirable that culture and differentiation be performed under the low ambient oxygen conditions comprise an ambient oxygen condition of between about 0.25% to about 18% oxygen. In another embodiment, the ambient oxygen conditions comprise between about 0.5% to about 15% oxygen. In still another embodiment, the low ambient oxygen conditions comprise between about 1% to about 10% oxygen. In further embodiments, the low ambient oxygen conditions comprise between about 1.5% to about 6% oxygen. Of course, these are exemplary ranges of ambient oxygen conditions to be used in culture and it should be understood that those of skill in the art will be able to employ oxygen conditions falling in any of these ranges generally or oxygen conditions between any of these ranges that mimics physiological oxygen conditions for the particular cells. Thus, one of skill in the art could set the oxygen culture conditions at 0.5%, 1%, 1.5%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, or any other oxygen condition between any of these figures.

One aspect of the invention relates to the timing (e.g. stage of cell culture) at which the chondrogenic cells are exposed to low oxygen (i.e. reduced oxygen tension) conditions. One skilled in the art will appreciate that the timing of the exposure of the chondrogenic cells to reduced oxygen tension will depend on the chondrogenic characteristics that are desired and which result from exposure to the altered environmental condition of low oxygen tension. chondrogenic cells may be exposed to reduced oxygen tension at any time during the in vitro culture of the stem cells. Chondrogenic cells may be exposed to reduced oxygen tension at times including, but not limited to, after collection of the chondrogenic cells as a tissue sample, during disaggregation of such tissue sample, during the primary culture of chondrogenic cells, during the in vitro expansion of the chondrogenic cells (e.g. over multiple cell passages), during priming (e.g. when chondrogenic cells are induced to assume a desired biological activity prior to injection into a subject), and combinations thereof. In some embodiments of the invention, chondrogenic cells are exposed to reduced oxygen tension during the in vitro culture of the chondrogenic cells. One skilled in the art will appreciate that there are various methods for culturing chondrogenic cells under low ambient oxygen conditions (i.e. reduced oxygen tension). For example, suitable processes, reagents and equipment for practicing the invention are disclosed in the following references, which are incorporated herein by reference: U.S. Pat. Nos. 6,759,242; 6,846,641; 6,610,540. Although these references disclose particular procedures and reagents, any low oxygen culture condition capable of expanding stern cells according to the invention may be used.

Chondrogenic cells can be exposed to low oxygen conditions under any methodology that permits the chondrogenic cells to attain an enhanced differentiation potential, proliferation rate, engraftment ability and/or in vivo tumor migratory ability. Specialized laboratory facilities may have completely enclosed environments in which the oxygen levels are controlled throughout a dedicated, isolated room. In such specialized areas, low oxygen levels can be maintained throughout the isolation, growth and differentiation of cells without interruption. Physiologic or low oxygen culturing conditions also can be maintained by using commercially-available chambers which are flushed with a pre-determined gas mixture (e.g., as available from Billups-Rothenberg, San Diego, Calif.). As an adjunct, medium can be flushed with the same gas mixture prior to cell feeding. In general, it is not possible to maintain physiologic or low oxygen conditions during cell feeding and passaging using these smatter enclosed units, and so, the time for these manipulations should be minimized as much as possible. Any sealed unit can be used for physiologic oxygen or low oxygen level culturing provided that adequate humidification, temperature, and carbon dioxide are provided. In addition to oxygen, the other gases for culture typically are about 5% carbon dioxide and the remainder is nitrogen, but optionally may contain varying amounts of nitric oxide (starting as low as 3 ppm), carbon monoxide and other gases, both inert and biologically active. Carbon dioxide concentrations typically range around 5% as noted above, hut may vary between 2-10%. Both nitric oxide and carbon monoxide are typically administered in very small amounts (i.e. in the ppm range), determined empirically or from the literature.

One aspect of the invention relates to the length of time that the chondrogenic cells are exposed to reduced oxygen tension. Under the invention, stem cells may be exposed to reduced oxygen tension for any amount of time that enhances the proliferation and differentiation of the chondrogenic cells as disclosed herein. This may be 1 or more hours, 3 or more hours, 6 or more hours, 12 or more hours, or the time may be continuous (e.g. the entire time that the chondrogenic cells are cultured in vitro). The temperature during the culture is typically reflective of core body temperature, or about 37.degree. C., but may vary between about 32 degrees centigrade and about 40 degrees centigrade.

In one embodiment, chondrogenic cells and/or chondrogenic progenitors are exposed to low dose carbon monoxide (CO) as a means of preconditioning prior to administration. CO may also be utilized as a preservative for storing cells prior to administration. It is an unexpected result that the inclusion of low dosage CO in the storage media maintains and/or enhances viability and activity. Thus, in this embodiment of the present invention, an effective amount of CO is bubbled into storage media before cells are placed in the media. or shortly thereafter.

In delivering CO concentrations ranging from about 0.001 to about 3,000 ppm pursuant to the present invention, gaseous compositions according to the present invention may be prepared by mixing commercially available compressed air containing CO (generally about 1% CO) with compressed air or gas containing a higher percentage of oxygen (including pure oxygen), and then mixing the gasses in a ratio which will produce a gas containing a desired amount of CO therein. Alternatively, compositions according to the present invention may be purchased pre-prepared from commercial gas companies. In a preferred embodiment, patients are exposed to oxygen (O.sub.2 at varying doses) and CO at a flow rate of about 12 liters/minute in a 3.70 cubic foot glass exposure chamber. To make a gaseous composition containing a pre-determined amount of CO, CO at a concentration of 1% (10,000 ppm) in compressed air is mixed with >98% O.sub.2 in a stainless steel mixing cylinder, concentrations delivered to the exposure chamber or tubing will be controlled. Because the flow rate is primarily determined by the flow rate of the O.sub.2 gas, only the CO flow is changed to generate the different concentrations delivered to the exposure chamber or tubing. A carbon monoxide analyzer (available from Interscan Corporation, Chatsworth, Calif.) is used to measure CO levels continuously in the chamber or tubing. Gas samples are taken by the analyzer through a portion the top of the exposure chamber of tubing at a rate of 1 liter/minute and analyzed by electrochemical detection with a sensitivity of about 1 ppb to 600 ppm. CO levels in the chamber or tubing are recorded at hourly intervals and there are no changes in chamber CO concentration once the chamber or tubing has equilibrated. CO is then delivered to the patient for a time (including chronically) sufficient to treat the condition and exert the intended pharmacological or biological effect.

In some embodiments of the invention, In the present invention, a “cartilage disease” refers to a disease caused as cartilage, cartilage tissue, and/or joint tissue (synovia, joint capsules, subchondral bones, etc.) are injured by a mechanical irritation or an inflammatory response, and comprises a disease associated with damaged cartilage. Such a cartilage disease may be, but is not limited to, degenerative arthritis, rheumatoid arthritis, a fracture, damaged muscle tissue, plantar fasciitis, lateral epicondylitis, calcific tendinitis, fracture nonunion, and a damaged joint due to trauma.

In addition, the present invention may provide a cell therapeutic agent comprising the aforementioned composition. A cell therapeutic agent is a cell and a tissue prepared through isolation from a human body, culturing, and a special treatment, and is a medicine used for therapeutic, diagnostic, and preventive purposes. It refers to a medicine used for therapeutic, diagnostic, and preventive purposes aimed at restoring the functions of a cell or tissue through a series of actions such as multiplying and screening a autologous, homologous, or heterologous living cell in vitro and modifying the biological characteristics of the cell in other way. The cell therapeutic agent may be directly injected into a joint of a patient or implanted together with a scaffold after three-dimensional (3D) culturing according to a well-known method, and the number of cells to be administered may be controlled, considering various associated factors such as the disease to be treated, the severity of the disease, the route of administration, and the body weight, age, and gender of the patient.

In addition, the composition or cell therapeutic agent of the present invention may be applied to a damaged portion of cartilage by being inoculated on a support for cartilage formulation. Such a support should be biocompatible, bioabsorbable, or capable of remodeling, and offer a framework for facilitating the growth of new tissue. In addition, the support should exhibit material and mechanical properties compatible with articular cartilage functions. A support providing an environment for 3D culturing affects the ultimate quality of the cartilage tissue prepared in a tissue-engineered manner as well as the proliferation and differentiation of the inoculated cells. Currently, various materials synthesized or derived from a natural material are used as a suitable support. Such supports take various forms such as a sponge, a gel, a fiber, and a microbead, and the most common form among them is a porous structure capable of improving the rate of cell adhesion and maintaining a large surface-tension-to-volume ratio. The composition or cell therapeutic agent of the present invention may be applied to a damaged portion of cartilage of a human or non-human organism, e.g., a non-human mammal such as a cow, monkey, bird, cat, mouse, rat, hamster, pig, dog, rabbit, sheep, and horse, to promote cartilage regeneration (differentiation), or be administered into a joint by injection for treating a cartilage disease.

In one embodiment, the present invention provides a pharmaceutical composition containing an active ingredient, that is a compound acts on pluripotent stem cell generated cartilage producing cell. Said active compound causes differentiation, proliferation, and maturation of such cells, enhance chondrocyte differentiation, induces chondrocyte proliferation, or increases cartilage matrix production; specifically, a compound that act on the prechondrocytes and/or pluripotent stem cells and induces at least 1, 2, 3, 4, or all of the following: (a) acceleration of prechondrocyte and/or mesenchymal stem cell differentiation; (b) acceleration of prechondrocyte and/or mesenchymal stem cell proliferation; (c) acceleration of prechondrocyte and/or mesenchymal stem cell maturation; (d) enhancement of chondrocyte differentiation; (e) chondrocyte proliferation; and (f) increased production of the cartilage matrix. Combinations of two or more of (a) to (f) are (a) and (b); (a) and (c); (a) and (d); (a) and (e); (a) and (f); (b) and (c); (b) and (d); (b) and (e); (b) and (f); (c) and (d); (c) and (e); (c) and (f); (d) and (e); (d) and (f); (e) and (f); (a), (b), and (c); (a), (b), and (d); (a), (b), and (e); (a), (b), and (f); (a), (c), and (d); (a), (c), and (e); (a), (c), and (f); (a), (d), and (e); (a), (d), and (f); (a), (e), and (f); (a), (b), (c), and (d); (a), (b), (c), and (e); (a), (b), (c), and (f); (a), (b), (d), and (e); (a), (b), (d), and (f); (a), (b), (e), and (f); (a), (c), (d), and (e); (a), (c), (d), and (f); (a), (c), (e), and (f); (a), (d), (e), and (f); (b), (c), (d), and (e); (b), (c), (d), and (f); (b), (c), (e), and (f); (b), (d), (e), and (f); (c), (d), (e), and (f); (a), (b), (c), (d), and (e); (a), (b), (c), (d), and (f); (a), (b), (c), (e), and (f); (a), (b), (d), (e), and (f); (a), (c), (d), (e), and (f); (b), (c), (d), (e), and (f); and (a), (b), (c), (d), (e), and (f). An example of such pharmaceutical composition is a pharmaceutical composition containing an active ingredient, that is a compound acts on the prechondrocytes and/or mesenchymal stem cells and accelerates cartilage differentiation, proliferation, and maturation of such cells, enhances chondrocyte differentiation, induces cartilage proliferation, or increases cartilage matrix production. An example of an active ingredient is a compound that acts on RANKL, transmits a signal to the prechondrocytes and/or mesenchymal stem cells, and accelerates differentiation, proliferation, and maturation of such cells, enhances chondrocyte differentiation, induces chondrocyte proliferation, or increases cartilage matrix production. When such compounds acts on RANKL, the animal origin of RANKL on which such compounds can act is not limited, and RANKL with any animal origin, such as human RANKL, mouse RANKL, or rat RANKL, can be targeted. The term “[compound] acts on RANKL” used herein refers to a situation in which a compound acts on RANKL and transmits a signal to the prechondrocytes and/or mesenchymal stem cells. For example, a compound may bind to RANKL and transmit a signal to prechondrocytes and/or pluripotent stem cells. Examples of the compound indicated the present invention, which acts on the prechondrocytes and/or mesenchymal stem cells and accelerates differentiation, proliferation, and maturation of such cells, enhances chondrocyte differentiation, induces chondrocyte proliferation, or increases cartilage matrix production include a mutant or a fragment peptide of RANK, a peptide structurally similar to RANK, a peptide structurally similar to a fragment peptide of RANK, a chemical substance structurally similar to RANK, and a chemical substance structurally similar to a fragment peptide of RANK. Examples of these compound include RANK, a mutant or a fragment peptide of RANK capable of acting on RANKL, a peptide structurally similar to RANK and capable of acting on RANKL, a peptide structurally similar to a fragment peptide of RANK and capable of acting on RANKL, a chemical substance structurally similar to RANK and capable of acting on RANKL, and a chemical substance structurally similar to a fragment peptide of RANK capable of acting on RANKL.

Other examples include the use of RNA interference to block RANK ligand expression and/or transcription. Therefore, the present invention provides a method of promoting the differentiation pluripotent stem cells and/or mesenchymal stem cells derived from said pluripotent stem cell into cartilage or treating a cartilage disease.

The invention provides several means of increasing efficacy of pluripotent stem cell generation. In one embodiment the invention provides downregulation of tumor suppressor genes as a means of inhibiting blocks associated with generation of pluripotent stem cells. For example, the utilization of RNA interference to temporarily induce suppression of p53 is one mechanism disclosed in the invention for generation of iPSCs. While it is known in the art that Oct4, Sox2, c-Myc and Klf4 are necessary to generate iPSCs, the invention describes means of utilizing other genes and or other approaches to increase efficacy.

Unfortunately, it is recognized that two of the four genes, c-Myc and Klf4, are oncogenic and may cause cancer. The efficiency of generating iPSCs is very low if c-Myc and Klf4 are not used. Cbx7 is downregulated during ESC differentiation and Cbx7 is never used to generate iPS cells generation. In one embodiment of the invention, it was surprisingly found that introduction of a Cbx family gene sequence but not c-Myc family gene sequence and Klf4 family gene sequence to somatic cells can efficiently reprogram differentiated cells into iPSCs that do not have oncogenic properties. Accordingly, the present disclosure provides a population of iPSCs, wherein the genetically modified somatic cells comprise a Cbx family gene sequence and one or more reprogramming factor sequences other than a cMyc family gene sequence and a Klf4 family gene sequence. Furthermore in one embodiment the invention suppression of p53 gene expression is used to increase efficacy of iPSC generation. For the purposes of iPSC generation, the invention teaches the use of dedifferentiated cells such as adult stem cells, however in some embodiments, somatic cells may be used. Somatic cells are cells that have differentiated sufficiently that they will not naturally generate cells of all three germ layers of the body, i.e. ectoderm, mesoderm and endoderm. They may differentiate to the point that they are capable of giving rise to cells of a specific lineage, e.g. adult non-pluripotent multipotent stem cells, e.g. mesenchymal stem cells, neural stem cells, cardiac stem cells, hepatic stem cells, and the like. Examples of somatic cells include the cells from ectodermal (e.g., keratinocytes), mesodermal (e.g., fibroblast), endodermal (e.g., pancreatic cells), or neural crest lineages (e.g. melanocytes). Certain embodiments include fibroblasts, keratinocytes, pancreatic beta cells, neurons, oligodendrocytes, astrocytes, hepatocytes, hepatic stem cells, cardiomyocytes, skeletal muscle cells, smooth muscle cells, hematopoietic cells, osteoclasts, osteoblasts, pericytes, vascular endothelial cells, Schwann cells, and the like. Somatic cells are reprogrammed using a Cbx family gene sequence and one or more reprogramming factor sequences (other than a cMyc family gene sequence and a Klf4 family gene sequence). Preferably, the Cbx family gene sequence is a nucleic acid sequence having at least 70% identical to the sequence of Cbx7. More preferably, the Cbx family gene sequence is Cbx7. The one or more reprogramming factor sequences are preferably Oct family gene sequence, a Sox family gene sequence, a Nanog family gene sequence, a Lin28 family gene sequence. In a preferred embodiment, the one or more reprogramming factor sequences include Oct family gene sequence and a Sox family gene sequence. Preferably, the Oct family gene sequence is a nucleic acid sequence having at least 70% identical to the amino acid sequence of Oct 3/4. Preferably, the Sox family gene sequence is a nucleic acid sequence having at least 70% identical to the amino acid sequence of Sox2. Preferably, the Nanog family gene sequence is a nucleic acid sequence having at least 70% identical to the amino acid sequence of Nanog. Preferably, the Lin28 family gene sequence is a nucleic acid sequence having at least 70% identical to the amino acid sequence of Lin28. The iPSC cells of the present disclosure are generated by a method comprising a step of (a) introducing somatic cells with a vector expressing a Cbx family gene and one or more vectors expressing one or more reprogramming factor genes rather than a cMyc family gene and a Klf4 family gene; and (b) culturing the resulting somatic cells of (a) under conditions which reprogram the resulting somatic cells of (a) to produce the iPSCs. Any appropriate vector expressing the reprogramming factors described herein may be used to introduce transgenes into somatic cells. Suitable vectors notably include plasmid vectors and viral vectors. Viral vectors can be replication-competent or -selective (e.g. engineered to replicate better or selectively in specific host cells), or can be genetically disabled so as to be replication-defective or replication-impaired. Typically, such vectors are commercially available (e.g. in Invitrogen, Stratagene, Amersham Biosciences, Promega, etc.) or available from depository institutions such as the American Type Culture Collection (ATCC, Rockville, Md.) or have been the subject of numerous publications describing their sequence, organization and methods of production, allowing the artisan to apply them. Representative examples of suitable viral vectors are generated from a variety of different viruses (e.g. retrovirus, adenovirus, adenovirus-associated virus (AAV), poxvirus, herpes virus, measles virus, foamy virus, alphavirus, vesicular stomatitis virus, lentivirus, etc). As described above, the term “viral vector” encompasses vector DNA, genomic DNA as well as viral particles generated therefrom, and especially infectious viral particles. In a preferred embodiment, a retrovirus vector or lentivirus vector. In a preferred embodiment of the present disclosure, a lentivirus is used to introduce transgenes into differentiated cells. Representative examples of suitable plasmid vectors include, without limitation, pREP4, pCEP4 (Invitrogen), pCI (Promega), pVAX (Invitrogen) and pGWiz (Gene Therapy System Inc.).

Vectors used for providing reprogramming factors to the subject cells as nucleic acids will typically comprise suitable promoters for driving the expression, that is, transcriptional activation, of the reprogramming factor nucleic acids. This may include ubiquitously acting promoters, for example, the CMV-b-actin promoter, or inducible promoters, such as promoters that are active in particular cell populations or that respond to the presence of drugs such as tetracycline. Subsequently, the genetically modified somatic cells harboring the Cbx family gene sequence and reprogramming factor sequences as described herein can transform to iPSCs by culturing and expanding the resulting somatic cells of under conditions which reprogram the resulting somatic cells to produce the iPSCs in the presence of feeder cells. The iPSCs are substantially isolated if it is mixed with carriers or diluents, such as culture medium, which will not interfere with its intended use. Alternatively, the iPSC of the invention may be present in a growth matrix or immobilized on a surface. For increasing efficacy of iPSC generation, in some embodiments the invention calls for generation of iPSC form peripheral blood or mobilized peripheral blood. Stem cell mobilization is a common procedure and usually involves administration of a mobilizing agent such as G-CSF. In one particular embodiment the invention generates iPSC by reprogramming blood cells (BCs) including peripheral mononuclear blood cells (PBMCs) into iPSCs (BC-iPSCs) and show that these iPSC lines are superior in terms of cytogenetic stability in comparison to their fibroblast-derived iPSC (Fib-iPSCs) lines obtained from public repositories or local clinics. In contrast to mature T or B cells, the alternative source of blood progenitors contain an intact genome. In addition, they can be expanded in culture conditions that favor the proliferation of myeloid cells or erythroid cells. Blood stem/progenitor cells express surface marker CD34 and reside in the stem cell niche. However, only about 1% stem/progenitor cells enter circulation each day and as a result, only 0.01-0.1% cells in PB are CD34+ cells. This population can be enriched by magnetic-activated cell sorting (MACS) or culture of MNCs for several days can be relied upon to expand CD34+ cells to a 5-20% purity, which can be used for reprogramming without further purification.

Other nucleated peripheral blood cells include granulocytes (mostly neutrophils), monocytes, T lymphocytes, B lymphocytes and a few progenitor cells. Focusing on these constitutes of blood can be achieved by depleting red blood cells and platelet using lysis buffer followed by multiple centrifugations. Ficoll gradient centrifugation can also be utilized to deplete both red blood cells and granulocytes, leading to the enrichment of mononuclear cells (MNCs). Against this backdrop, reprogramming with exogenously expressed factors is notoriously inefficient and requires multiple cell cycles to achieve pluripotency. As such, primary granulocytes, monocytes and B lymphocytes are among the most difficult cells to be reprogrammed due to the lack of reliable protocols to expand these cells. Primary success in this area includes Epstein-Barr virus immortalized lymphoblastoid B cells can be readily expanded in ex vivo culture and thus be reprogrammed to pluripotency. In view of the above, of great interest is reprogramming of the non-T cell component of blood. Existing techniques are largely unable to reprogram this population from isolated peripheral blood mononuclear cells (PBMCs). More specifically, PBMCs are any peripheral blood cell having a round nucleus. This includes lymphocytes (T cells, B cells, NK cells), monocytes, dendritic cells. Lymphocytes are Small (5-10 μm) and Medium (10-18 μm) and constitute 70-90% of PBMCs. Of these cells, 70-85% CD3+ T cells (40-70% of PBMCs), CD4 Helper T cells (25-60% of PBMCs), typically with CD4 to CD8 ratio of 2:1, CD8 “Cytotoxic” compartment T cells (5-30% of PBMCs). The remaining compartment includes 5-20% B Cells (up to 15% of PBMCs) and 5-20% NK Cells (up to 15% of PBMCs). Monocytes are 16-25 am and 10-30% of PBMCs (macrophages). Dendritic cells: 1-2% of PBMCs. These described approaches allow for use of peripheral blood as a readily accessible resource for cellular reprogramming with superior properties in genomic and karyotype stability avoiding environmental insults for which mutations or other forms of structural alteration would otherwise be therapeutic materials derived therein. As described, the Inventors have established improved techniques for highly efficient, reproducible reprogramming using non-integrating episomal plasmid vectors, including generation of iPSCs from blood cells, including whole blood and peripheral blood, the resulting reprogrammed pluripotent cells described herein as “BC-iPSCs”. Generally, different approaches for non-integrative reprogramming span at least categories: 1) integration-defective viral delivery, 2) episomal delivery, 3) direct RNA delivery, 4) direct protein delivery and 5) chemical induction. As described further herein, the adoption of episomal vectors allows for generation of iPSCs substantially free of the vectors used in their production, as episomal or similar vectors do not encode sufficient viral genome sufficient to give rise to infection or a replication-competent virus. At the same time, these vectors do possess a limited degree of self-replication capacity in the beginning somatic host cells. This self-replication capacity provides a degree of persistent expression understood to be beneficial in allowing the dedifferentiation process to initiate take hold in a target host cell.

One example of a plasmid vector satisfying these criteria includes the Epstein Barr oriP/Nuclear Antigen-1 (“EBNA1”) combination, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. More specifically, persistent expression of reprogramming factor encoded in an oriP/EBNA1 vector occurs across multiple cell divisional cycles. Sufficiently high levels of reprogramming factors across several cell divisions allows for successful reprogramming even after only one infection. While sustained expression of reprogramming factors is understood to be beneficial during initial programming stages, otherwise unlimited constitutive expression would hamper subsequent stages of the reprogramming process. For example, unabated expression of reprogramming factors would interfere with subsequent growth, development, and fate specification of the host cells.

At the same time, a further benefit is the eventual removal of the reprogramming factor transgenes, as a small portion of episomes is lost per cell cycle. This is due to the asymmetric replication capacity of the host cell genome and episomal self-replication and it is estimated that approximately 0.5% of vector is lost per generation. Gradual depletion of plasmids during each cell division is inevitable following propagation leading to a population of integration-free iPSCs. The persistent, yet eventual abrogation of reprogramming factor expression on oriP/EBNA1 is highly coincident with the needs for different stages of the reprogramming process and eliminates the need for further manipulation steps for excision of the reprogramming factors, as has been attempted through use of transposons and excisable polycistronic lentiviral vector elements. Although oriP/EBNA1 has been applied by others in reprogramming studies, the reported efficiencies are extremely low (as few as 3 to 6 colonies per million cells nucleofected), which may be due, in-part, to reliance on large plasmids encoding multiple reprogramming factors (e.g., more than 12 kb), negatively impacting transfection efficiency.

In addition to these choices in vector designs, the specific combinations of reprogramming factors implemented in the literature have varied. As mentioned, reprogramming factors that have been used include pluripotency-related genes Oct-4, Sox-2, Lin-28, Nanog, Sa114, Fbx-15 and Utf-1. These factors, traditionally are understood be normally expressed early during development and are involved in the maintenance of the pluripotent potential of a subset of cells that will constituting the inner cell mass of the pre-implantation embryo and post-implantation embryo proper. Their ectopic expression of is believed to allow the establishment of an embryonic-like transcriptional cascade that initiates and propagates an otherwise dormant endogenous core pluripotency program within a host cell. Certain other reprogramming determinants, such as Tert, Klf-4, c-Myc, SV40 Large T Antigen (“SV40LT”) and short hairpin RNAs targeting p53 (“shRNA-p53”) have been applied. There determinants may not be potency-determining factors in and of themselves, but have been reported to provide advantages in reprogramming. For example, TERT and SV40LT are understood to enhance cell proliferation to promote survival during reprogramming, while others such as short hairpin targeting of p53 inhibit or eliminate reprogramming barriers, such as senescence and apoptosis mechanisms. In each case, an increase in both the speed and efficiency of reprogramming is observed. In addition, microRNAs (“miRNAs”) are also known to influence pluripotency and reprogramming, and some miRNAs from the miR-290 cluster have been applied in reprogramming studies. For example, the introduction of miR-291-3p, miR-294 or miR-295 into fibroblasts, along with pluripotency-related genes, has also been reported to increase reprogramming efficiency. These factors may also be introduced by non-integrating viruses such as sendai.

While various vectors and reprogramming factors in the art appear to present multiple ingredients capable of establishing reprogramming in cells, a high degree of complexity occurs when taking into account the stoichiometric expression levels necessary for successful reprogramming to take hold. For example, somatic cell reprogramming efficiency is reportedly fourfold higher when Oct-4 and Sox2 are encoded in a single transcript on a single vector in a 1:1 ratio, in contrast to delivering the two factors on separate vectors. The latter case results in a less controlled uptake ratio of the two factors, providing a negative impact on reprogramming efficiency. One approach towards addressing these obstacles is the use of polycistronic vectors, such as inclusion of an internal ribosome entry site (“IRES”), provided upstream of transgene(s) that is distal from the transcriptional promoter. This organization allows one or more transgenes to be provided in a single reprogramming vector, and various inducible or constitutive promoters can be combined together as an expression cassette to impart a more granular level of transcriptional control for the plurality of transgenes. These more specific levels of control can benefit the reprogramming process considerably, and separate expression cassettes on a vector can be designed accordingly as under the control of separate promoters.

Although there are advantages to providing such factors via a single, or small number of vectors, upper size limitations on eventual vector size do exist, which can stymie attempts to promote their delivery in a host target cell. For example, early reports on the use of polycistronic vectors were notable for extremely poor efficiency of reprogramming, sometimes occurring in less than 1% of cells, more typically less than 0.1%. These obstacles are due, in-part, to certain target host cells possessing poor tolerance for large constructs (e.g., fibroblasts), or inefficient processing of IRES sites by the host cells. Moreover, positioning of a factor in a vector expression cassette affects both its stoichiometric and temporal expression, providing an additional variable impacting reprogramming efficiency. Thus, some improved techniques can rely on multiple vectors each encoding one or more reprogramming factors in various expression cassettes. Under these designs, alteration of the amount of a particular vector for delivery provides a coarse, but relatively straightforward route for adjusting expression levels in a target cell.

A further advantage of the techniques described herein is the use of defined media conditions for the reprogramming process, including the use of ESC media and/or E7 media. While certain additives may be present to spur the reprogramming process (e.g., L-Ascorbic Acid, Transferrin, Sodium Bicarbonate, Insulin, Sodium Selenite and/or bFGF), no serum or animal components are used. In some instances, there may be further benefits in altering the chemical and/or atmospheric conditions under which reprogramming will take place. For example, as the pre-implantation embryo is not vascularized and hypoxic (similar to bone marrow stem-cell niches) reprogramming under hypoxic conditions of 5% 02, instead of the atmospheric 21% 02, may further provide an opportunity to increase the reprogramming efficiency. Similarly, chemical induction techniques have been used in combination with reprogramming, particularly histone deacetylase (HDAC) inhibitor molecule, valproic acid (VPA), which has been found wide use in different reprogramming studies. At the same time, other small molecules such as MAPK kinase (MEK)-ERK (“MEK”) inhibitor PD0325901, transforming growth factor beta (“TGF-3”) type I receptor ALK4, ALK5 and ALK7 inhibitor SB431542 and the glycogen synthase kinase-3 (“GSK3”) inhibitor CHIR99021 have been applied for activation of differentiation-inducing pathways (e.g. BMP signaling), coupled with the modulation of other pathways (e.g. inhibition of the MAPK kinase (MEK)-ERK pathway) in order to sustain self-renewal. Other small molecules, such as Rho-associated coiled-coil-containing protein kinase (“ROCK”) inhibitors, such as Y-27632 and thiazovivin (“Tzv”) have been applied in order to promote survival and reduce vulnerability of pSCs to cell death, particularly upon single-cell dissociation.

In addition to the choice of delivery vectors, reprogramming factor combinations, and conditions for reprogramming, further variations must consider the nature of the host target cell for reprogramming. To date, a wide variety of cells have served as sources for reprogramming including fibroblasts, stomach and liver cell cultures, human keratinocytes, adipose cells, and frozen human monocyte. Clearly, there is a wide and robust potential for dedifferentiation across many tissue sources. Nevertheless, it is widely understood that depending on the donor cell type, reprogramming is achieved with different efficiencies and kinetics. For example, although fibroblasts remain the most popular donor cell type for reprogramming studies, other types of cells such as human primary keratinocytes transduced with Oct-4, Sox-2, Klf-4 and c-Myc have been reported to reprogram 100 times more efficiently and two-fold faster. Additionally, some other cell types, such as cord blood cells, may only require a subset of reprogramming factors, such as Oct-4 and Sox-2 for dedifferentiation to take hold, while neural progenitor cells may only require Oct-4. Without being bound to any particular theory, it is believed that differences in reprogramming efficiency and/or reprogramming factor requirements of specific host cells result from high endogenous levels of certain reprogramming factors and/or intrinsic epigenetic states that are more amenable to reprogramming. Although these many other sources have been used across studies for the generation of iPSCs, mononuclear cells (MNCs) from peripheral blood (PB) are a highly attractive host cell candidate due to convenience and features as an almost unlimited resource for cell reprogramming. PB cells in particular are relatively easy to isolate (e.g., blood draw) compared to isolation from other sources such as fibroblasts (e.g., skin biopsy). These cells do not require laborious culturing and propagation prior to reprogramming, thereby reducing the overall time from which reprogramming iPSCs can be obtained.

Following successful reprogramming, clonal selection allows for generation of pluripotent stem cell lines. Ideally, such cells possess requisite morphology (i.e., compact colony, high nucleus to cytoplasm ratio and prominent nucleolus), self-renewal capacity for unlimited propagation in culture (i.e., immortal), and with the capability to differentiate into all three germ layers (e.g., endoderm, mesoderm and ectoderm). Further techniques to characterize the pluripotency of a given population of cells include injection into an immunocompromised animal, such as a severe combined immunodeficient (“SCID”) mouse, for formation of teratomas containing cells or tissues characteristic of all three germ layers.

Described herein is a composition of blood cell derived induced pluripotent stem cells (“BC-iPSCs”). In certain embodiments, the composition of blood cell derived induced pluripotent stem cells includes cells generated by providing a quantity of blood cells, delivering a quantity of reprogramming factors into the blood cells, culturing the blood cells in a reprogramming media for at least 4 days, wherein delivering the reprogramming factors, and culturing generates the blood cells derived induced pluripotent stem cells. In certain embodiments, the blood cells are T-cells. In other embodiments, the blood cells are non-T-cells. In other embodiments, the blood cells are mononuclear cells (MNCs), including for example peripheral blood mononuclear cells (PBMCs). In other embodiments, the cells are primary granulocytes, monocytes and B lymphocytes. In certain embodiments, the reprogramming factors are Oct-4, Sox-2, Klf-4, c-Myc, Lin-28, SV40 Large T Antigen (“SV40LT”), and short hairpin RNAs targeting p53 (“shRNA-p53”). In other embodiments, these reprogramming factors are encoded in a combination of vectors including pEP4 E02S ET2K, pCXLE-hOCT3/4-shp53-F, pCXLE-hSK, pCXLE-hUL and pCXWB-EBNA1. This includes, for example, using about 0.5-1.0 ug pCXLE-hOCT3/4-shp53, 0.5-1.0 ug pCXLE-hSK, 0.5-1.0 ug pCXLE-UL, about 0.25-0.75 ug pCXWB-EBNA1 and 0.5-1.0 ug pEP4 E02S ET2K. This includes, for example, using 0.83 ug pCXLE-hOCT3/4-shp53, 0.83 ug pCXLE-hSK, 0.83 ug pCXLE-UL, 0.5 ug pCXWB-EBNA1 and 0.83 ug pEP4 E02S ET2K, wherein the stoichiometric ratio of SV40LT (encoded in pEP4 E02S ET2K) and EBNA-1 (encoded in pCXWB-EBNA1) supports the reprogramming of non-T cell component of blood, including peripheral blood mononuclear cells. In certain other embodiments, the reprogramming media includes PD0325901, CHIR99021, HA-100, and A-83-01. In other embodiments, the culturing the blood cells in a reprogramming media is for 4-30 days. In various embodiments, the blood cells are plated on a treated cell culture surface after delivering a quantity of reprogramming factors. In various embodiments, treatment includes plating of feeder cells, such as mouse embryonic fibroblasts. In other embodiments, treatment includes coating with extracellular matrix proteins. In various embodiment, extracellular matrix proteins include laminin.

In various embodiments, the BC-iPSCs are capable of serial passaging as a cell line. In various embodiments, the BC-iPSCs possess genomic stability. Genomic stability can be ascertained by various techniques known in the art. For example, G-band karyotyping can identify abnormal cells lacking genomic stability, wherein abnormal cells possess about 10% or more mosaicism, or one or more balanced translocations of greater than about 5, 6, 7, 8, 9, 10 or more Mb. Alternatively, genomic stability can be measured using comparative genomic hybridization (aCGH) microarray, comparing for example, BC-iPSCs against iPSCs from a non-blood cell source such as fibroblasts. Genomic stability can include copy number variants (CNVs), duplications/deletions, and unbalanced translocations. In various embodiments, BC-iPSCs exhibit no more than about 3, 4, 5, 6, 7, 8, 9, 10, 11, 12, 13, 15, 16, 17, 18, 19, or 20 Mb average size of amplification and deletion. In various embodiments, BC-iPSCs exhibit no more than about 20-30 Mb average size of amplification and deletion. In various embodiments, BC-iPSCs exhibit no more than about 30-40 Mb average size of amplification and deletion. In various embodiments, BC-iPSCs exhibit no more than about 40-50 Mb average size of amplification and deletion. In various embodiments, the average number of acquired de novo amplification and deletions in BC-iPSCs is less than about 5, 4, 3, 2, or 1. For example, de novo amplification and deletions in fib-iPSCs are at least two-fold greater than in PBMC-iPSCs.

In different embodiments, reprogramming factors can also include one or more of following: Oct-4, Sox-2, Klf-4, c-Myc, Lin-28, SV40LT, shRNA-p53, nanog, Sa114, Fbx-15, Utf-1, Tert, or a Mir-290 cluster microRNA such as miR-291-3p, miR-294 or miR-295. In different embodiments, the reprogramming factors are encoded by a vector. In different embodiments, the vector can be, for example, a non-integrating episomal vector, minicircle vector, plasmid, retrovirus (integrating and non-integrating) and/or other genetic elements known to one of ordinary skill. In different embodiments, the reprogramming factors are encoded by one or more oriP/EBNA1 derived vectors. In different embodiments, the vector encodes one or more reprogramming factors, and combinations of vectors can be used together to deliver one or more of Oct-4, Sox-2, Klf-4, c-Myc, Lin-28, SV40LT, shRNA-p53, nanog, Sa114, Fbx-15, Utf-1, Tert, or a Mir-290 cluster microRNA such as miR-291-3p, miR-294 or miR-295. For example, oriP/EBNA1 is an episomal vector that can encode a vector combination of multiple reprogramming factors, such as pCXLE-hUL, pCXLE-hSK, pCXLE-hOCT3/4-shp53-F, pEP4 EO2S T2K and pCXWB-EBNA1. In other embodiments, the reprogramming factors are delivered by techniques known in the art, such as nuclefection, transfection, transduction, electrofusion, electroporation, microinjection, cell fusion, among others. In other embodiments, the reprogramming factors are provided as RNA, linear DNA, peptides or proteins, or a cellular extract of a pluripotent stem cell. In various embodiments, the reprogramming media is embryonic stem cell (ESC) media. In various embodiments, the reprogramming media includes bFGF. In various embodiments, the reprogramming media is E7 media. In various embodiments, the reprogramming E7 media includes L-Ascorbic Acid, Transferrin, Sodium Bicarbonate, Insulin, Sodium Selenite and/or bFGF In different embodiments, the reprogramming media comprises at least one small chemical induction molecule. In different embodiments, the at least one small chemical induction molecule comprises PD0325901, CHIR99021, HA-100, A-83-01, valproic acid (VPA), SB431542, Y-27632 or thiazovivin (“Tzv”). In different embodiments, culturing the BCs in a reprogramming media is for at least 4, 5, 6, 7, 8, 9, 10, 11, 12, 13, 14, 15, 16, 17, 18, 19, 20, 21, 22, 23, 24, 25, 26, 27, 28, 29, or 30 days. In certain embodiments, the BC-iPSCs are derived from blood cells previously isolated from a subject, by for, example, drawing a blood sample from the subject. In other embodiments, the blood cells are isolated from a subject possessing a disease mutation. For example, subjects possessing any number of mutations, such as autosomal dominant, recessive, sex-linked, can serve as a source of blood cells to generate BC-iPSCs possessing said mutation. In other embodiments, the disease mutation is associated with a neurodegenerative disease, disorder and/or condition. In other embodiments, the disease mutation is associated with an inflammatory bowel disease, disorder, and/or condition. In various embodiments, the BC-iPSCs possess features of pluripotent stem cells. Some exemplary features of pluripotent stem cells including differentiation into cells of all three germ layers (ectoderm, endoderm, mesoderm), either in vitro or in vivo when injected into an immunodeficient animal, expression of pluripotency markers such as Oct-4, Sox-2, nanog, TRA-1-60, TRA-1-81, SSEA4, high levels of alkaline phosphatase (“AP”) expression, indefinite propagation in culture, among other features recognized and appreciated by one of ordinary skill. Also described herein is an efficient method for generating induced pluripotent stem cells, including providing a quantity of cells, delivering a quantity of reprogramming factors into the cells, culturing the cells in a reprogramming media for at least 4 days, wherein delivering the reprogramming factors, and culturing generates induced pluripotent stem cells. In certain embodiments, the cells are primary culture cells. In other embodiments, the cells are blood cells (BCs). In certain embodiments, the blood cells are T-cells. In other embodiments, the blood cells are non-T-cells. In other embodiments, the cells are mononuclear cells (MNCs), including for example peripheral blood mononuclear cells (PBMCs). In other embodiments, the cells are primary granulocytes, monocytes and B lymphocytes. In certain embodiments, the reprogramming factors are Oct-4, Sox-2, Klf-4, c-Myc, Lin-28, SV40 Large T Antigen (“SV40LT”), and short hairpin RNAs targeting p53 (“shRNA-p53”). In other embodiments, these reprogramming factors are encoded in a combination of vectors including pEP4 E02S ET2K, pCXLE-hOCT3/4-shp53-F, pCXLE-hSK, pCXLE-hUL and pCXWB-EBNA1. This includes, for example, using about 0.5-1.0 ug pCXLE-hOCT3/4-shp53, 0.5-1.0 ug pCXLE-hSK, 0.5-1.0 ug pCXLE-UL, about 0.25-0.75 ug pCXWB-EBNA1 and 0.5-1.0 ug pEP4 E02S ET2K. This includes, for example, using 0.83 ug pCXLE-hOCT3/4-shp53, 0.83 ug pCXLE-hSK, 0.83 ug pCXLE-UL, 0.5 ug pCXWB-EBNA1 and 0.83 ug pEP4 E02S ET2K, wherein the stoichiometric ratio of SV40LT (encoded in pEP4 E02S ET2K) and EBNA-1 (encoded in pCXWB-EBNA1) supports the reprogramming of non-T cell component of blood, including peripheral blood mononuclear cells. In various embodiments, the reprogramming media is embryonic stem cell (ESC) media. In various embodiments, the reprogramming media includes bFGF. In various embodiments, the reprogramming media is E7 media. In various embodiments, the reprogramming E7 media includes L-Ascorbic Acid, Transferrin, Sodium Bicarbonate, Insulin, Sodium Selenite and/or bFGF. In different embodiments, the reprogramming media comprises at least one small chemical induction molecule. In certain other embodiments, the reprogramming media includes PD0325901, CHIR99021, HA-100, and A-83-01. In other embodiments, the culturing the blood cells in a reprogramming media is for 4-30 days. In various embodiments, the BC-iPSCs are capable of serial passaging as a cell line. In various embodiments, the BC-iPSCs possess genomic stability. Genomic stability can be ascertained by various techniques known in the art. For example, G-band karyotyping can identify abnormal cells lacking genomic stability, wherein abnormal cells possess about 10% or more mosaicism, or one or more balanced translocations of greater than about 5, 6, 7, 8, 9, 10 or more Mb. Alternatively, genomic stability can be measured using comparative genomic hybridization (aCGH) microarray, comparing for example, BC-iPSCs against iPSCs from a non-blood cell source such as fibroblasts. Genomic stability can include copy number variants (CNVs), duplications/deletions, and unbalanced translocations. In various embodiments, BC-iPSCs exhibit no more than about 3, 4, 5, 6, 7, 8, 9, 10, 11, 12, 13, 15, 16, 17, 18, 19, or 20 Mb average size of amplification and deletion. In various embodiments, BC-iPSCs exhibit no more than about 20-30 Mb average size of amplification and deletion. In various embodiments, BC-iPSCs exhibit no more than about 30-40 Mb average size of amplification and deletion. In various embodiments, BC-iPSCs exhibit no more than about 40-50 Mb average size of amplification and deletion. In various embodiments, the average number of acquired de novo amplification and deletions in BC-iPSCs is less than about 5, 4, 3, 2, or 1. For example, de novo amplification and deletions in fib-iPSCs are at least two-fold greater than in PBMC-iPSCs. In various embodiments, the methods produces iPSC cell lines collectively exhibiting about 20%, 15%, 10%, 5% or less abnormal karyotypes over 4-8, 9-13, 13-17, 17-21, 21-25, or 29 or more passages when serially passaged as a cell line. In different embodiments, reprogramming factors can also include one or more of following: Oct-4, Sox-2, Klf-4, c-Myc, Lin-28, SV40LT, shRNA-p53, nanog, Sa114, Fbx-15, Utf-1, Tert, or a Mir-290 cluster microRNA such as miR-291-3p, miR-294 or miR-295. In different embodiments, the reprogramming factors are encoded by a vector. In different embodiments, the vector can be, for example, a non-integrating episomal vector, minicircle vector, plasmid, retrovirus (integrating and non-integrating) and/or other genetic elements known to one of ordinary skill. In different embodiments, the reprogramming factors are encoded by one or more oriP/EBNA1 derived vectors. In different embodiments, the vector encodes one or more reprogramming factors, and combinations of vectors can be used together to deliver one or more of Oct-4, Sox-2, Klf-4, c-Myc, Lin-28, SV40LT, shRNA-p53, nanog, Sa114, Fbx-15, Utf-1, Tert, or a Mir-290 cluster microRNA such as miR-291-3p, miR-294 or miR-295. For example, oriP/EBNA1 is an episomal vector that can encode a vector combination of multiple reprogramming factors, such as pCXLE-hUL, pCXLE-hSK, pCXLE-hOCT3/4-shp53-F, pEP4 EO2S T2K and pCXWB-EBNA1. In other embodiments, the reprogramming factors are delivered by techniques known in the art, such as nuclefection, transfection, transduction, electrofusion, electroporation, microinjection, cell fusion, among others. In other embodiments, the reprogramming factors are provided as RNA, linear DNA, peptides or proteins, or a cellular extract of a pluripotent stem cell. In certain embodiments, the cells are treated with sodium butyrate prior to delivery of the reprogramming factors. In other embodiments, the cells are incubated or 1, 2, 3, 4, or more days on a tissue culture surface before further culturing. This can include, for example, incubation on a Matrigel coated tissue culture surface. In other embodiments, the reprogramming conditions include application of norm-oxygen conditions, such as 5% 02, which is less than atmospheric 21% 02. In various embodiments, the reprogramming media is embryonic stem cell (ESC) media. In various embodiments, the reprogramming media includes bFGF. In various embodiments, the reprogramming media is E7 media. In various embodiments, the reprogramming E7 media includes L-Ascorbic Acid, Transferrin, Sodium Bicarbonate, Insulin, Sodium Selenite and/or bFGF. In different embodiments, the reprogramming media comprises at least one small chemical induction molecule. In different embodiments, the at least one small chemical induction molecule comprises PD0325901, CHIR99021, HA-100, A-83-01, valproic acid (VPA), SB431542, Y-27632 or thiazovivin (“Tzv”). In different embodiments, culturing the BCs in a reprogramming media is for at least 4, 5, 6, 7, 8, 9, 10, 11, 12, 13, 14, 15, 16, 17, 18, 19, 20, 21, 22, 23, 24, 25, 26, 27, 28, 29, or 30 days. Efficiency of reprogramming is readily ascertained by one of many techniques readily understood by one of ordinary skill. For example, efficiency can be described by the ratio between the number of donor cells receiving the full set of reprogramming factors and the number of reprogrammed colonies generated. Measuring the number donor cells receiving reprogramming factors can be measured directly, when a reporter gene such as GFP is included in a vector encoding a reprogramming factor. Alternatively, indirect measurement of delivery efficiency can be provided by transfecting a vector encoding a reporter gene as a proxy to gauge delivery efficiency in paired samples delivering reprogramming factor vectors. Further, the number of reprogrammed colonies generated can be measured by, for example, observing the appearance of one or more embryonic stem cell-like pluripotency characteristics such as alkaline phosphatase (AP)-positive clones, colonies with endogenous expression of transcription factors Oct or Nanog, or antibody staining of surface markers such as Tra-1-60. In another example, efficiency can be described by the kinetics of induced pluripotent stem cell generation. For example, efficiency can include producing cell lines of normal karyotype, including the method producing iPSC cell lines collectively exhibiting about 20%, 15%, 10%, 5% or less abnormal karyotypes over 4-8, 9-13, 13-17, 17-21, 21-25, or 29 or more passages when serially passaged as a cell line.

In some embodiments of this invention, the pluripotency of a cell is tested in vivo by examining its capability of growing into teratoma containing all three germ cells. In another embodiment, the pluripotency is tested by the expression of certain markers in cultured cells ex vivo. In yet another embodiment, the pluripotency of a cell is tested by its contribution to the development of an embryo into a living organism. The pluripotent stem cells are injected into the inner cell mass (ICM) of an embryonic blastocyst, which is then implanted into the uterus of a female organism and developing into a fetus. The term “chimerism” refers to the contribution of the stem cells and their progenies to all three germ layers that give rise to various tissues in a living organism.

There are numerous methods of differentiating the induced cells into a more specialized cell type. Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases, the differentiation occurs in vivo. In one embodiment, neural stem cells may be generated by culturing the induced cells as floating aggregates in the presence of noggin, or other bone morphogenetic protein antagonist according to some embodiments, the composition of the present invention may be formulated with an excipient, carrier or vehicle including, but not limited to, a solvent. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide the desired bulk, consistency, etc., when combined with an active agent and other components of a given composition. Suitable pharmaceutically acceptable carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like. Such carrier solutions also can contain buffers, diluents and other suitable additives. The term “buffer” as used herein refers to a solution or liquid whose chemical makeup neutralizes acids or bases without a significant change in pH. Examples of buffers envisioned by the present invention include, but are not limited to, Dulbecco's phosphate buffered saline (PBS), Ringer's solution, 5% dextrose in water (D5W), normal/physiologic saline (0.9% NaCl). According to some embodiments, the infusion solution is isotonic to subject tissues. According to some embodiments, the infusion solution is hypertonic to subject tissues. Compositions of the present invention that are for parenteral administration can include pharmaceutically acceptable carriers such as sterile aqueous solutions, non-aqueous solutions in common solvents such as alcohols, or solutions in a liquid oil base. The compositions of the present invention may be administered parenterally in the form of a sterile injectable aqueous or oleaginous suspension. The term “parenteral” or “parenterally” as used herein refers to introduction into the body by way of an injection (i.e., administration by injection), including, but not limited to, infusion techniques. According to some embodiments, parenteral administration includes but is not limited to intravascular delivery (meaning into a blood vessel), intravenous delivery (meaning into a vein), intra-arterial delivery (meaning into an artery), intraosseous delivery (meaning into the bone marrow), intramuscular delivery (meaning into a muscle), subcutaneous delivery (meaning under the skin), cardiac delivery (meaning into the heart, myocardium), etc. The delivery route may vary and depend on the origin of degenerative diseases. In a preferred embodiment, the delivery route for treating degenerative conditions in central nervous system is intracranial injection.

In one embodiment of the invention, the generation of pluripotent stem cells is provided in a manner which is scalable to production of commercial-grade cellular products. Although several methodologies have been reported for generation of pluripotent stem cells, these are characterized by low level of cellular dedifferentiation, as well as problems with large-scale expansion. In one embodiment the invention provides conditions for enhancing the generation of pluripotent stem cells in part by creating environments that resemble embryonic stem cell development. Accordingly target cells to be dedifferentiated are grown in conditions that possess the multiple cells associated with the three dimensional embryonic environment. Specifically, in some embodiments amniotic membrane stem cells are utilized as feeder layers or as a cellular composite to support the cells which are to be dedifferentiated. Cells included in culture to support dedifferentiation include mesenchymal stem cells, monocytes, B cells and NKT cells. In some embodiments bone marrow endothelial cells are utilized to expand various cells to be dedifferentiated. It is occasionally desired to utilized defined liquid media in order to avoid reproducibility issues associated with fetal calf serum lot to lot variability. Accordingly, the invention provides the utilization of liquid media containing various histone deacetylase inhibitors to allow for expansion of stem cells without differentiation. Useful histone deacetylase inhibitors include valproic acid, trichostatin A, sodium phenylbutyrate and sulforaphane. The invention discloses culture medium being serum-free and devoid of non-human contaminants comprising ascorbic acid at a concentration range of about 200-6000 μg/ml, basic fibroblast growth factor (bFGF) at a concentration range of about 5-2000 ng/ml, serum replacement and a lipid mixture, wherein the culture medium is capable of maintaining pluripotent stem cells in an undifferentiated state and with a stable karyotype for at least 40 passages in the absence of feeder cell support. In some embodiments the invention provides for feeder layers as well. The expansion of stem cells may be performed utilizing various types of liquid culture media as a base. For example, the GIBCO™ KNOCKOUT™ Serum Replacement (Gibco-Invitrogen Corporation, Grand Island, N.Y. USA, Catalogue No. 10828028) is a defined serum-free formulation optimized to grow and maintain undifferentiated ES cells in culture. It should be noted that the formulation of GIBCO™ Knockout™ Serum Replacement includes Albumax (Bovine serum albumin enriched with lipids) which is from an animal source (International Patent Publication No. WO 98/30679 to Price, P. J. et al According to some embodiments of the invention, the concentration of GIBCO™ KNOCKOUT™ Serum Replacement in the culture medium is in the range of from about 1% [volume/volume (v/v)] to about 50% (v/v), e.g., from about 5% (v/v) to about 40% (v/v), e.g., from about 5% (v/v) to about 30% (v/v), e.g., from about 10% (v/v) to about 30% (v/v), e.g., from about 10% (v/v) to about 25% (v/v), e.g., from about 10% (v/v) to about 20% (v/v), e.g., about 10% (v/v), e.g., about 15% (v/v), e.g., about 20% (v/v), e.g., about 30% (v/v). For expansion of iPSC there may be various cytokines added to said media. One such cytokine is leukemia inhibitory factor, which may be added at concentrations of 1 pg/ml to 500 ng/ml, more preferably 10 pg/ml to 200 ng/ml, more preferably 100 pg/ml to 100 ng/ml. There exist other commercially available serum replacement is the B27 supplement without vitamin A which is available from Gibco-Invitrogen, Corporation, Grand Island, N.Y. USA, Catalogue No. 12587-010. The B27 supplement is a serum-free formulation which includes d-biotin, fatty acid free fraction V bovine serum albumin (BSA), catalase, L-carnitine HCl, corticosterone, ethanolamine HCl, D-galactose (Anhyd.), glutathione (reduced), recombinant human insulin, linoleic acid, linolenic acid, progesterone, putrescine-2-HCl, sodium selenite, superoxide dismutase, T-3/albumin complex, DL alpha-tocopherol and DL alpha tocopherol acetate. However, the use of B27 supplement is limited since it includes albumin from an animal source. In one embodiment leukemia inhibitory factor is added to said B27 supplement. In conditions in which animal-free media are desirable, such as in cases in which generation of autoantibodies, alloantibodies or xenoantibodies are to be avoided, it may be preferably to utilizing animal free media, which is sometimes referred to as “xeno-free”. Non-limiting examples of commercially available xeno-free serum replacement compositions include the premix of ITS (Insulin, Transferrin and Selenium) available from Invitrogen corporation (ITS, Invitrogen, Catalogue No. 51500-056); Serum replacement 3 (Sigma, Catalogue No. S2640) which includes human serum albumin, human transferring and human recombinant insulin and does not contain growth factors, steroid hormones, glucocorticoids, cell adhesion factors, detectable Ig and mitogens. According to some embodiments of the invention, the xeno-free serum replacement formulations ITS (Invitrogen corporation) and SR3 (Sigma) are diluted in a 1 to 100 ratio in order to reach a ×1 working concentration. According to some embodiments of the invention the culture medium is capable of maintaining pluripotent stem cell in a proliferative, pluripotent and undifferentiated state for at least about 5 passages, at least about 10 passages, at least about 15 passages, at least about 20 passages, at least about 22 passages, at least about 25 passages, at least about 30 passages, at least about 35 passages, at least about 40 passages, at least about 45 passages, at least about 50 passages and more. According to some embodiments of the invention the culture medium is capable of expanding the pluripotent stem cells in an undifferentiated state. One of the important aspects of the current invention is the proliferation or expansion of pluripotent stem cells without their differentiation. The number of pluripotent stem cells over the culturing period (by at least about 5%, 10%, 15%, 20%, 30%, 50%, 100%, 200%, 500%, 1000%, and more). It will be appreciated that the number of pluripotent stem cells which can be obtained from a single pluripotent stem cell depends on the proliferation capacity of the pluripotent stem cell. The proliferation capacity of a pluripotent stem cell can be calculated by the doubling time of the cell (i.e., the time needed for a cell to undergo a mitotic division in the culture) and the period the pluripotent stem cell culture can be maintained in the undifferentiated state (which is equivalent to the number of passages multiplied by the days between each passage). According to some embodiments of the invention, the culture medium of some embodiments of the invention is capable of supporting expansion of a single pluripotent stem cell (e.g., hESC or human iPS cell) or a population of pluripotent stem cells by at least 223 (i.e., 8×106) within about one month, e.g., at least 224 (i.e., 16.7×106) within about one month. According to some embodiments of the invention the serum-free and xeno-free culture medium comprises basic fibroblast growth factor (bFGF), transforming growth factor beta-3 (TGFβ3) and ascorbic acid, wherein a concentration of the ascorbic acid in the culture medium is at least 50 μg/ml and wherein the culture medium is capable of maintaining pluripotent stem cells in an undifferentiated state in the absence of feeder cell support. Ascorbic acid (also known as vitamin C) is a sugar acid (C6H806; molecular weight 176.12 grams/mole) with antioxidant properties. The ascorbic acid used by the culture medium of some embodiments of the invention can be a natural ascorbic acid, a synthetic ascorbic acid, an ascorbic acid salt (e.g., sodium ascorbate, calcium ascorbate, potassium ascorbate), an ester form of ascorbic acid (e.g., ascorbyl palmitate, ascorbyl stearate), a functional derivative thereof (a molecule derived from ascorbic acid which exhibits the same activity/function when used in the culture medium of the invention), or an analogue thereof (e.g., a functional equivalent of ascorbic acid which exhibits an activity analogous to that observed for ascorbic acid when used in the culture medium of the invention). Non-limiting examples of ascorbic acid formulations which can be used in the culture medium of some embodiments of the invention include L-ascorbic acid and ascorbic acid 3-phosphate. Ascorbic acid can be obtained from various manufacturers such as Sigma, St Louis, Mo., USA (e.g., Catalogue numbers: A2218, A5960, A7506, A0278, A4403, A4544, A2174, A2343, 95209, 33034, 05878, 95210, 95212, 47863, 01-6730, 01-6739, 255564, A92902, W210901). As mentioned, the concentration of ascorbic acid in the culture medium is at least about 50 μg/ml. According to some embodiments of the invention, the ascorbic acid can be used in a range of concentrations such as from about 50 μg/ml to about 50 mg/ml, e.g., from about 50 μg/ml to about 5 mg/ml, e.g., from about 50 μg/ml to about 1 mg/ml, e.g., from about 100 μg/ml to about 800 μg/ml, e.g., from about 200 μg/ml to about 800 μg/ml, e.g., from about 300 μg/ml to about 700 μg/ml, e.g., from about 400 μg/ml to about 600 μg/ml, e.g., from about 450 μg/ml to about 550 μg/ml. According to some embodiments of the invention the concentration of ascorbic acid in the culture medium is at least about 75 μg/ml, e.g., at least about 100 μg/ml, e.g., at least about 150 μg/ml, e.g., at least about 200 μg/ml, e.g., at least about 250 μg/ml, e.g., at least about 300 μg/ml, e.g., at least about 350 μg/ml, e.g., at least about 400 μg/ml, e.g., at least about 450 μg/ml, e.g., about 500 pg/ml. Basic fibroblast growth factor (also known as bFGF, FGF2 or FGF-β) is a member of the fibroblast growth factor family. The bFGF used in the culture medium of some embodiments of the invention can be a purified, a synthetic or a recombinantly expressed bFGF protein [(e.g., human bFGF polypeptide GenBank Accession No. NP-001997.5 (SEQ ID NO:31); human bFGF polynucleotide GenBank Accession No. NM-002006.4 (SEQ ID NO:32). It should be noted that for the preparation of a xeno-free culture medium the bFGF is preferably purified from a human source or is recombinantly expressed as is further described hereinbelow. bFGF can be obtained from various commercial sources such as Cell Sciences®, Canton, Mass., USA (e.g., Catalogue numbers CRF001A and CRF001B), Invitrogen Corporation products, Grand Island N.Y., USA (e.g., Catalogue numbers: PHG0261, PHG0263, PHG0266 and PHG0264), ProSpec-Tany TechnoGene Ltd. Rehovot, Israel (e.g., Catalogue number: CYT-218), and Sigma, St Louis, Mo., USA (e.g., catalogue number: F0291). According to some embodiments the concentration of bFGF in culture medium is in the range from about 1 ng/ml to about 10 μg/ml, e.g., from about 2 ng/ml to about 1 μg/ml, e.g., from about 1 ng/ml to about 500 ng/ml, e.g., from about 2 ng/ml to about 500 ng/ml, e.g., from about 5 ng/ml to about 250 ng/ml, e.g., from about 5 ng/ml to about 200 ng/ml, e.g., from about 5 ng/ml to about 150 ng/ml, e.g., about 10 ng/ml, e.g., about 20 ng/ml, e.g., about 30 ng/ml, e.g., about 40 ng/ml, e.g., about 50 ng/ml, e.g., about 60 ng/ml, e.g., about 70 ng/ml, e.g., about 80 ng/ml, e.g., about 90 ng/ml, e.g., about 100 ng/ml, e.g., about 110 ng/ml, e.g., about 120 ng/ml, e.g., about 130 ng/ml, e.g., about 140 ng/ml, e.g., about 150 ng/ml. According to some embodiments of the invention the concentration of bFGF in the culture medium is at least about 1 ng/ml, at least about 2 ng/ml, at least about 3 ng, at least about 4 ng/ml, at least about 5 ng/ml, at least about 6 ng/ml, at least about 7 ng, at least about 8 ng/ml, at least about 9 ng/ml, at least about 10 ng/ml, at least about 15 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 70 ng/ml, at least about 80 ng/ml, at least about 90 ng/ml, at least about 95 ng/ml, e.g., about 100 ng/ml. Transforming growth factor beta-3 (TGFβ3) is involved in the control of proliferation, differentiation, and other functions in many cell types, acts in inducing transformation and as a negative autocrine growth factor. TGFβ3 can be obtained from various commercial sources such as R&D Systems Minneapolis Minn., USA. According to some embodiments of the invention, the concentration of TGFβ3 in the culture medium is in the range of about 0.05 ng/ml to about 1 μg/ml, e.g., from 0.1 ng/ml to about 1 μg/ml, e.g., from about of about 0.5 ng/ml to about 100 ng/ml. According to some embodiments of the invention, the concentration of TGFβ3 in the culture medium is at least about 0.5 ng/ml, e.g., at least about 0.6 ng/ml, e.g., at least about 0.8 ng/ml, e.g., at least about 0.9 ng/ml, e.g., at least about 1 ng/ml, e.g., at least about 1.2 ng/ml, e.g., at least about 1.4 ng/ml, e.g., at least about 1.6 ng/ml, e.g., at least about 1.8 ng/ml, e.g., about 2 ng/ml.

According to some embodiments of the invention, the culture medium comprises bFGF at a concentration range of about 0.1 ng/ml to about 500 ng/ml, TGFβ3 at a concentration range of about 0.1 ng/ml to about 20 ng/ml, and ascorbic acid at a concentration range of about 50 μg/ml to about 5000 μg/ml. According to some embodiments of the invention, the culture medium of some embodiments of the invention comprises bFGF at a concentration range of about 5 ng/ml to about 150 ng/ml, TGFβ3 at a concentration range of about 0.5 ng/ml to about 5 ng/ml, and ascorbic acid at a concentration range of about 400 μg/ml to about 600 μg/ml. According to some embodiments of the invention, the concentration of the lipid mixture in the culture medium is from about 0.5% [volume/volume (v/v)] to about 3% v/v, e.g., from about 0.5% v/v to about 2% v/v, e.g., from about 0.5% v/v to about 1% v/v, e.g., about 1% v/v. According to some embodiments of the invention, the culture medium of some embodiments of the invention comprises bFGF at a concentration range of about 0.1 ng/ml to about 500 ng/ml, TGFβ3 at a concentration range of about 0.1 ng/ml to about 20 ng/ml, ascorbic acid at a concentration range of about 50 μg/ml to about 5000 μg/ml, xeno-free serum replacement and a lipid mixture. Non-limiting examples of xeno-free and serum-free culture media which comprise TGFβ3, bFGF and ascorbic acid at a concentration of at least 50 μg/ml and which can be used to maintain pluripotent stem cells in a proliferative and undifferentiated states include the HA75 and HA78 culture media. According to some embodiments of the invention, the culture medium further comprises sodium bicarbonate. Sodium bicarbonate can be obtained from Biological Industries, Beit HaEmek, Israel. According to some embodiments of the invention, the concentration of sodium bicarbonate in the culture medium is from about 5% to about 10%, e.g., from about 6% to about 9%, e.g., from about 7% to about 8%, e.g., about 7.5%. The present inventors uncovered that pluripotent stem cells can be maintained in a proliferative, pluripotent and undifferentiated state for at least 15 passages when cultured in a serum-free and xeno-free culture medium which comprises bFGF and ascorbic acid but does not comprise a TGFβ isoform. According to some embodiments of the invention, the culture medium comprises no more than 1 ng/ml of the TGFβ isoform, e.g., no more than 0.5 ng/ml, e.g., no more than 0.1 ng/ml, e.g., no more than 0.05 ng/ml, e.g., no more than 0.01 ng/ml of the TGFβ isoform. According to some embodiments of the invention the culture medium comprises ascorbic acid at a concentration range of about 400-600 μg/ml and basic fibroblast growth factor (bFGF) at a concentration range of about 50-200 ng/ml. According to some embodiments of the invention the culture medium the culture medium which comprises ascorbic acid at a concentration range of about 400-600 μg/ml and basic fibroblast growth factor (bFGF) at a concentration range of about 50-200 ng/ml is capable of maintaining pluripotent stem cells in an undifferentiated state in the absence of feeder cell support. According to some embodiments of the invention, the concentration of ascorbic acid in the culture medium is between about 410 μg/ml to about 590 μg/ml, between about 420 μg/ml to about 580 μg/ml, between about 450 μg/ml to about 550 μg/ml, between about 460 μg/ml to about 540 μg/ml, between about 470 μg/ml to about 530 μg/ml, between about 490 μg/ml to about 520 μg/ml, e.g., between about 490 μg/ml to about 510 μg/ml, e.g., about 500 μg/ml. According to some embodiments of the invention, the concentration of bFGF in the culture medium is between about 50 ng/ml to about 200 ng/ml, between about 60 ng/ml to about 190 ng/ml, between about 70 ng/ml to about 180 ng/ml, between about 80 ng/ml to about 170 ng/ml, between about 90 ng/ml to about 160 ng/ml, between about 90 ng/ml to about 150 ng/ml, between about 90 ng/ml to about 130 ng/ml, between about 90 ng/ml to about 120 ng/ml, e.g., about 100 ng/ml. According to some embodiments of the invention, the concentration of bFGF in the culture medium is about 50, about 55, about 60, about 65, about 70, about 80, about 85, about 90, about 95, about 100, about 105, about 110, about 115, about 120, about 125, about 130, about 135, about 140, about 145, about 150, about 160, about 165, about 170, about 175, about 180, about 185, about 190, about 195, about 200 ng/ml. According some embodiments of the invention the culture medium which comprises ascorbic acid at a concentration range of about 400-600 μg/ml and basic fibroblast growth factor (bFGF) at a concentration range of about 50-200 ng/ml, further comprises xeno-free serum replacement. According to some embodiments of the invention, the culture medium which comprises ascorbic acid at a concentration range of about 400-600 μg/ml and basic fibroblast growth factor (bFGF) at a concentration range of about 50-200 ng/ml, further comprises a lipid mixture. According to some embodiments of the invention, the culture medium comprises bFGF at a concentration of about 50-200 ng/ml and ascorbic acid at a concentration of about 400-600 μg/ml is devoid of sodium-bicarbonate. According to some embodiments of the invention, the culture medium comprises bFGF at a concentration of about 50-200 ng/ml and ascorbic acid at a concentration of about 400-600 μg/ml, xeno-free serum replacement at a concentration of about 1% and lipid mixture at a concentration of about 1%.

A non-limiting example of a xeno-free, serum-free, and TGFβ isoform-free culture medium which comprises ascorbic acid at a concentration range of about 400-600 μg/ml, bFGF at a concentration range of about 50-200 ng/ml, xeno-free serum replacement and a lipid mixture and which is capable of maintaining pluripotent stem cells such as hESCs and human iPS cells in a proliferative and undifferentiated state for at least 21 passages in the absence of feeder cell support is the HA77 culture medium or a culture medium similar to the HA77 medium but which is devoid of sodium bi-carbonate such as a culture medium which consists of DMEM/F12 (94%) (Biological Industries, Israel, Sigma Israel), L-glutamine 2 mM (Invitrogen corporation, Sigma, Israel), ascorbic acid 500 μg/ml (Sigma, Israel), bFGF-100 ng (Invitrogen corporation), SR3-1% (Sigma, Israel), and defined lipid mixture 1% (Invitrogen corporation, Sigma, Israel). The present inventors have uncovered novel serum-free and highly defined culture media, which can maintain pluripotent stem cells in a proliferative, pluripotent and undifferentiated state in two-dimensional and three-dimensional (i.e., a suspension culture) systems in the absence of feeder cell support.

As used herein the phrase “suspension culture” refers to a culture in which the pluripotent stem cells are suspended in a medium rather than adhering to a surface.

According to some embodiments of the invention the serum-free culture medium which can maintain pluripotent stem cells in a proliferative, pluripotent and undifferentiated state in two-dimensional and three-dimensional culture systems in the absence of feeder cell support comprises basic fibroblast growth factor (bFGF) at a concentration range of about 50-200 ng/ml. According to some embodiments of the invention the culture medium comprises between about 55-190 ng/ml, e.g., between about 60-190 ng/ml, e.g., between about 70-180 ng/ml, e.g., between about 80-160 ng/ml, e.g., between about 90-150 ng/ml, e.g., between about 90-140 ng/ml, e.g., between about 90-130 ng/ml, e.g., between about 90-120 ng/ml, e.g., between about 90-110 ng/ml, e.g., between about 95-105 ng/ml, e.g., about 100 ng/ml. According to some embodiments of the invention the culture medium which comprises bFGF between about 50-200 ng/ml further comprises serum replacement.

A non-limiting example of a culture medium which comprises bFGF at a concentration between about 50-200 ng/ml is the YF100 medium which comprises a basic medium (e.g., DMEM/F12, 85%), serum replacement (15%), bFGF (100 ng/ml), L-glutamine (2 mM), β-mercaptoethanol (0.1 mM) and non-essential amino acid stock (1%). According to some embodiments of the invention the serum-free culture medium which can maintain pluripotent stem cells in a proliferative, pluripotent and undifferentiated state in two-dimensional and three-dimensional culture systems in the absence of feeder cell support consists of a basic medium, ascorbic acid at a concentration range of about 50 μg/ml to about 500 μg/ml, bFGF at a concentration range between about 2 ng/ml to about 20 ng/ml, L-glutamine, and serum replacement.

According to some embodiments of the invention the serum-free culture medium which can maintain pluripotent stem cells in a proliferative, pluripotent and undifferentiated state in two-dimensional and three-dimensional culture systems in the absence of feeder cell support consists of a basic medium, ascorbic acid at a concentration range of about 50 μg/ml to about 500 μg/ml, bFGF at a concentration range between about 2 ng/ml to about 20 ng/ml, L-glutamine, serum replacement and a lipid mixture.

The serum replacement can be any xeno-free serum replacement (devoid of animal contaminants) at a concentration range from 1-20% depending on the serum replacement used. For example, if the SR3 serum replacement is used then it concentration in the medium is about 1%. Non-limiting examples of such a culture medium include the modified HA13(a) medium [DMEM/F12 (95%), L-glutamine 2 mM, ascorbic acid 500 μg/ml, bFGF-4 ng, and SR3-1%]; the modified HA13(b) medium [DMEM/F12 (95%), L-glutamine 2 mM, ascorbic acid 500 μg/ml, bFGF-4 ng, SR3-1% and a lipid mixture (1%)]; the modified HA13(c) medium [DMEM/F12 (95%), L-glutamine 2 mM, ascorbic acid 50 μg/ml, bFGF-4 ng, and SR3-1%]; and the modified HA13(d) medium [DMEM/F12 (95%), L-glutamine 2 mM, ascorbic acid 50 μg/ml, bFGF-4 ng, SR3-1% and a lipid mixture (1%)]. These culture media were capable of maintaining pluripotent stem cells (e.g., hESCs and hips cells) in a proliferative, pluripotent and undifferentiated state for at least 20 passages when cultured in a two-dimensional (e.g., on a feeder-layer free culture system; data not shown) and for at least 20 passages when cultured on a three-dimensional culture system (e.g., suspension culture without adherence to an external substrate, cell encapsulation or to protein carrier; data not shown). According to some embodiments of the invention the serum-free culture medium which can maintain pluripotent stem cells in a proliferative, pluripotent and undifferentiated state in two-dimensional and three-dimensional culture systems in the absence of feeder cell support comprises an IL6RIL6 chimera at a concentration range of about 50-200 picogram per milliliter (pg/ml). According to some embodiments of the invention, the concentration of the IL6RIL6 chimera in the culture medium is in the range from about 55 pg/ml to about 195 pg/ml, e.g., from about 60 pg/ml to about 190 pg/ml, e.g., from about 65 pg/ml to about 185 pg/ml, e.g., from about 70 pg/ml to about 180 pg/ml, e.g., from about 75 pg/ml to about 175 pg/ml, e.g., from about 80 pg/ml to about 170 pg/ml, e.g., from about 85 pg/ml to about 165 pg/ml, e.g., from about 90 pg/ml to about 150 pg/ml, e.g., from about 90 pg/ml to about 140 pg/ml, e.g., from about 90 pg/ml to about 130 pg/ml, e.g., from about 90 pg/ml to about 120 pg/ml, e.g., from about 90 pg/ml to about 110 pg/ml, e.g., from about 95 pg/ml to about 105 pg/ml, e.g., from about 98 pg/ml to about 102 pg/ml, e.g., about 100 pg/ml. According to some embodiments of the invention, the IL6RIL6 chimera-containing culture medium further comprises bFGF. According to some embodiments of the invention, concentration of bFGF in the IL6RIL6 chimera-containing culture medium is in the range of from about 1 ng/ml to about 10 μg/ml, e.g., from about 2 ng/ml to about 1 μg/ml, e.g., from about 2 ng/ml to about 500 ng/ml, e.g., from about 5 ng/ml to about 150 ng/ml, e.g., from about 5 ng/ml to about 100 ng/ml, e.g., from about 5 ng/ml to about 80 ng/ml, e.g., from about 5 ng/ml to about 50 ng/ml, e.g., from about 5 ng/ml to about 30 ng/ml, e.g., about 5 ng/ml, e.g., about 10 ng/ml, e.g., about 15 ng/ml, e.g., about 20 ng/ml. According to some embodiments of the invention, the IL6RIL6 chimera-containing culture medium further comprises serum replacement. According to some embodiments of the invention, the concentration of KNOCKOUT™ Serum Replacement in the IL6RIL6 chimera-containing culture medium is in the range from about 1% (v/v) to about 50% (v/v), e.g., from about 5% (v/v) to about 40% (v/v), e.g., from about 5% (v/v) to about 30% (v/v), e.g., from about 10% (v/v) to about 30% (v/v), e.g., from about 10% (v/v) to about 25% (v/v), e.g., from about 10% (v/v) to about 20% (v/v), e.g., about 15% (v/v). According to some embodiments of the invention, the culture medium comprises IL6RIL6 chimera at a concentration range of about 50-200 pg/ml, bFGF at a concentration range of about 5-50 ng/ml and serum replacement at a concentration of about 5-40%. According to some embodiments of the invention, the serum-free culture medium which can maintain pluripotent stem cells in a proliferative, pluripotent and undifferentiated state in two-dimensional and three-dimensional culture systems in the absence of feeder-cells support comprises LIF at a concentration of at least 2000 units/ml. Leukemia inhibitory factor (LIF) is a pleiotropic cytokine which is involved in the induction of hematopoietic differentiation, induction of neuronal cell differentiation, regulator of mesenchymal to epithelial conversion during kidney development, and may also have a role in immune tolerance at the maternal-fetal interface. The LIF used in the culture medium of some embodiments of the invention can be a purified, synthetic or recombinantly expressed LIF protein [e.g., human LIF polypeptide GenBank Accession No. NP-002300.1. It should be noted that for the preparation of a xeno-free culture medium LIF is preferably purified from a human source or is recombinantly expressed. Recombinant human LIF can be obtained from various sources such as Chemicon, USA (Catalogue No. LIF10100) and AbD Serotec (MorphoSys US Inc, Raleigh, N.C. 27604, USA). Murine LIF ESGRO® (LIF) can be obtained from Millipore, USA (Catalogue No. ESG1107).

According to some embodiments of the invention, the concentration of LIF in the culture medium is from about 2000 units/ml to about 10,000 units/ml, e.g., from about 2000 units/ml to about 8,000 units/ml, e.g., from about 2000 units/ml to about 6,000 units/ml, e.g., from about 2000 units/ml to about 5,000 units/ml, e.g., from about 2000 units/ml to about 4,000 units/ml. According to some embodiments of the invention, the concentration of LIF in the culture medium is at least about 2000 units/ml, e.g., at least about 2100 units/ml, e.g., at least about 2200 units/ml, e.g., at least about 2300 units/ml, e.g., at least about 2400 units/ml, e.g., at least about 2500 units/ml, e.g., at least about 2600 units/ml, e.g., at least about 2700 units/ml, e.g., at least about 2800 units/ml, e.g., at least about 2900 units/ml, e.g., at least about 2950 units/ml, e.g., about 3000 units/ml. According to some embodiments of the invention, the LIF-containing culture medium further comprises bFGF. The concentration of bFGF in the LIF-containing culture medium is in the range of about 0.1 ng/ml to about 10 μg/ml, e.g., from about 2 ng/ml to about 1 μg/ml, e.g., from about 2 ng/ml to about 500 ng/ml, e.g., from about 5 ng/ml to about 150 ng/ml, e.g., from about 5 ng/ml to about 100 ng/ml, e.g., from about 5 ng/ml to about 80 ng/ml, e.g., from about 5 ng/ml to about 50 ng/ml, e.g., from about 5 ng/ml to about 30 ng/ml, e.g., about 5 ng/ml, e.g., about 10 ng/ml, e.g., about 15 ng/ml, e.g., about 20 ng/ml. According to some embodiments of the invention, the LIF-containing culture medium further comprises serum replacement. According to some embodiments of the invention, the culture medium comprises LIF at a concentration of about 2000-10,000 units/ml, bFGF at a concentration range from about 0.1 ng/ml to about 10 μg/ml and KNOCKOUT™ Serum Replacement at a concentration range from about 1% (v/v) to about 50% (v/v). According to some embodiments of the invention, the culture medium comprises LIF at a concentration of about 2000-5,000 units/ml, bFGF at a concentration of about 5-50 ng/ml and serum replacement at a concentration of about 5-30%. According to some embodiments of the invention, the ingredients included in the culture medium of some embodiments of the invention are substantially pure, with a tissue culture and/or a clinical grade. According to an aspect of some embodiments of the invention there is provided a cell culture which comprises the pluripotent stem cell of some embodiments of the invention and the culture medium of some embodiments of the invention. According to an aspect of some embodiments of the invention cell culture is feeder cells free (e.g., being devoid of feeder cells or feeder cell conditioned medium). According to some embodiments of the invention the pluripotent stem cells which are included in the cell culture of some embodiments of the invention exhibit a stable karyotype (chromosomal stability) during the culturing period, e.g., for at least 2 passages, e.g., at least 4 passages, e.g., at least 8 passages, e.g., at least 15 passages, e.g., at least 20 passages, e.g., at least 25 passages, e.g., at least 30 passages, e.g., at least 35 passages, e.g., at least 40 passages, e.g., at least 45 passages, e.g., at least 50 passages.

According to some embodiments of the invention, the cell culture of the invention exhibit a doubling time of at least 20 hours, e.g., a doubling time which is between 20 to 40 hours (e.g., about 36 hours), thus representing a non-tumorigenic, genetically stable pluripotent stem cells (e.g., hESCs and iPS cells). According to some embodiments of the invention, the cell culture of the invention is characterized by at least 40%, at least 50%, at least 60%, e.g., at least 70%, e.g., at least 80%, e.g., at least 85%, e.g., at least 90%, e.g., at least 95% of undifferentiated pluripotent stem cells. According to an aspect of some embodiments of the invention, there is provided a method of expanding and maintaining pluripotent stem cells in a pluripotent and undifferentiated state. Furthermore, for generation of iPSC for creating MSC to use in orthopedic conditions, we present multiple culture conditions and media, which permit the indefinite culture and robust proliferation of primate pluripotent stem cells in an undifferentiated state with continued expression of characteristic pluripotency markers. Also, the media described are prepared in the complete absence of both feeder cells and conditioned medium. As described here, the defined culture conditions and media are suitable for use with human pluripotent stem cells. Pluripotent cells express one or more pluripotent cell-specific marker, such as Oct-4, SSEA-3, SSEA-4, Tra 1-60, Tra 1-81. They include, but are not limited to human ES cells (e.g., H1, H7, H9 and H14), iPS cells (e.g., iPS-Foreskin and iPS-IMR90), and vector-free iPS cells (e.g., iPS-DF19-9, iPS-DF4-3, and iPS-DF6-9), which are all available through WiCell® International Stem Cell (WISC) Bank (Madison, Wis.). iPS cells are described in Yu J. et al. Science, 318(5858), pp. 1917-1920 (2007) and vector-free iPS cells are described in Yu J. et al. Science, 324(5928), pp. 797-801 (2009), both of these references are incorporated by reference here in their entirety. For some primate pluripotent stem cells, including iPS and vector-free iPS cells, certificate of analyses for primate pluripotent stem cells cultured on growth matrix using the fully defined medium disclosed here is available on the WISC website. Additional pluripotent stem cell lines include, but are not limited to, disease model cell lines and genetically modified lines containing marker genes. In many embodiments of the invention, the culture conditions and media are entirely free of non-human animal products and all proteins used are of human origin. The development of these media and culture conditions make possible the derivation and maintenance of human pluripotent stem cell lines in defined and controlled conditions without direct or indirect exposure to non-human animal cells of any kind. Also, the media and culture conditions described here enable the derivation of new lines of human pluripotent stem cells which have never been exposed to non-human cells or a to medium in which animal cells were cultured. In one embodiment, the medium is free of animal products or proteins. This medium has been demonstrated to support undifferentiated pluripotent stem cell proliferation through at least twenty-five passages, which is firm evidence that it will support such cultures indefinitely. A suitable medium is capable of supporting the derivation of new human ES and iPS cell lines, and derived using the media described herein after as “new lines”. These lines have passed through more than ten passages in culture. In the past, use of conditioned medium has sometimes been referred to as creating “feeder-free” culture conditions. This phrase is a misnomer, since feeder cells of some type are still needed to condition the “conditioned medium.” As described here, culture conditions permit the “feeder-independent” culture of human pluripotent stem cells. By “feeder-independent” it is meant that no feeder cells of any kind, human or animal, are needed anywhere in the process and are neither required for culture nor to condition the medium. Feeder-independent conditions do not require feeder cells at all for any purpose. A defined and humanized medium for the culture and proliferation of human pluripotent stem cells typically includes salts, vitamins, lipids, an energy source such as glucose, minerals, amino acids, growth factors and other components. As a supplement to support cell growth, stem cell media have included serum from one source or another. Also, previously it has been reported that the addition of FGF plus a serum replacement additive permits the cultivation of human pluripotent stem cells without serum. The serum replacement additive can be a commercially available product sold for that purpose or can be a formulated mixture of proteins, including but not limited to serum albumin, vitamins, minerals, a transferrin or a transferrin substitute, and insulin or an insulin substitute. The albumin, insulin and transferrin may be recombinant proteins. This serum replacement additive may also be supplemented with, but is not limited to, selenium and a mixture of lipids. Preferably, a defined serum replacement mix is used in lieu of serum from any source in culturing human pluripotent stem cells, to avoid variation in serum constituents and to use media that are as defined as possible. Other growth factors which have been found to be advantageous additives to the culture medium include, but are not limited to, gamma-aminobutyric acid (GABA), pipecolic acid (PA), lithium chloride (LiCI) and transforming growth factor beta (TGFβ). It has been found that TGFβ may not be needed when increasing levels of FGF are added to the medium. It is envisioned that other lithium salts can substitute for LiCI in the cell culture medium. These may include lithium salts, wherein the anion includes, but is not limited to, chloride, bromide, carbonate, citrate, sulfate, or other biologically compatible monovalent anion (see, for example, US 2004/0028656 and WO 2008/055224). To avoid the need for a fibroblast feeder layer, previously thought to be necessary to maintain human pluripotent stem cells (ES and iPS cells) in an undifferentiated state, it is reported here that combining the use of higher concentrations of FGF (10 to 1000 ng/ml) together with the use of GABA, PA, Li and TGFβ, will enable a medium to support long term (at least three passages, and suitably 170 passages) undifferentiated stem cell growth. The combination of these additives has been found to be sufficient to maintain the culture of human pluripotent stem cells in an undifferentiated state indefinitely without exposure to either feeder cells or conditioned media. These additives are demonstrably sufficient. However, all of them may not be necessary for every medium formulation. By selective deletion of these additives, it may be empirically determined if one or more of them is not required to achieve this result for a given medium. However, it is clear that the combination is sufficient to enable a variety of media that will support the long-term culture and proliferation of undifferentiated human pluripotent stem cells without feeder cells or conditioned medium.

These additives are subject to some variation. For example, GABA is believed to interact with the GABA receptor and the scientific literature includes the identification of several molecules which are agonists of that same receptor and might be substituted for GABA in the medium as an equivalent. It is also believed that PA also interacts with the GABA receptor. While both PA and GABA were found to be helpful in the medium at the concentrations used here, it is also envisioned that one or the other of these constituents could be increased in concentration to obviate the need for the other. The FGF in higher concentrations (40 to 100 ng/ml) seems to obviate the need for feeder cells. The preferred FGF is bFGF, also referred to as FGF2, but other FGFs, including at least FGF4, FGF9, FGF17, and FGF18, will suffice for this purpose as well. Other FGFs may also work, even if at higher concentrations, which can be empirically determined by researchers. Initial subjective screens performed by the inventors identified several growth factors, chosen based on the receptors expressed by the human pluripotent stem cells, as having positive effects on undifferentiated proliferation. Of these, bFGF, LiCI, GABA, PA, and TGFβ were ultimately included in TeSR1. For each of the multiple cell lines tested, the proliferation rate and the percentage of cells maintaining expression of characteristic human pluripotent stem cell markers were higher in TeSR1 than in control cells cultured in fibroblast-conditioned medium and removal of any one of these five factors decreased culture performance. Some of these data are illustrated in FIG. 6, which shows that cultures grown in media with any one of these constituents omitted exhibited a lesser percentage of cells which remained undifferentiated as compared to cultures with all five of these medium constituents included. Note that Oct-4, SSEA-1, SSEA-4, Tra 1-60 and Tra 1-81 are all cell surface markers or transcription factors (Oct-4) which are used to track the differentiation status of stem cells. FIG. 4 illustrates similar trials in which it was demonstrated that, over multiple passages, undifferentiated cell proliferation was the highest when all these constituents together were in the culture medium.

The inventors also found it advantageous to include in the culture vessel of human pluripotent stem cells a biological matrix. One such material that has been used previously is Matrigel™, which is an artificial basement matrix of mouse cell origin, which is supplied as a commercial product free of mouse cells. However, the use of Matrigel introduces into the culture a material which is poorly defined and includes material of murine origin. Accordingly, also described here is how to create a biological matrix of human proteins that can substitute completely for the Matrigel. This matrix is composed of a blend of four human proteins: collagen isolated from human placenta, fibronectin isolated from human plasma, vitronectin isolated from human plasma or from a recombinant source, and laminin isolated from human placenta. Other extracellular matrices may be suitable for use in the present invention, which include, but are not limited to, proteoglycan, entactin, heparan sulfate, and the like, alone or in various combinations. Other suitable extracellular matrices may include, but are not limited to, Geltrex™. The major components of Geltrex™ matrix include laminin, collagen IV, entactin, and heparin sulfate proteoglycan. Also suitable are human plasma fibronectin, recombinant human plasma fibronectin, human cellular fibronectin, recombinant human cellular fibronectin, and synthetic fibronectin in combination with at least one other matrix, such as collagen. Preferred matrices of the present invention include collagen, fibronectin, vitronectin, and laminin derived matrices.

The combination of these four human proteins is sufficient, but the use of all four may not be necessary to support the growth and culture of human pluripotent stem cells, as demonstrated by the experimental results depicted in FIG. 10. For example, the use of such a matrix without one of vitronectin, fibronectin, or laminin, but including the other three matrix proteins, does support the culture of pluripotent stem cells, with some loss of purity in the state of differentiation of the ES or iPS cell culture. Likewise, it is envisioned that the use of such a matrix without two of vitronectin, fibronectin or laminin, does support the culture of pluripotent stem cells, with some loss of purity in the state of differentiation of the ES or iPS cell culture. Suitable matrix protein combinations include collagen and fibronectin, collagen and vitronectin, and collagen and laminin. The method of making the matrix for pluripotent stem cell growth is described in the examples below. To arrive at the above-listed medium additives, the inventors methodically tested over 80 individual media components, including growth factors. While some of the additives seemed, at least for a few passages, to support the growth of human pluripotent stem cells in culture, many failed in subsequent passages to maintain the pluripotent stem cells in an undifferentiated state. The inventors were able to identify combinations of specific growth factors useful in the medium described in the examples below. Also, through methodical testing, the inventors were able to investigate the effects of varying the concentration of β-mercaptoethanol (BME) in the TeSR1 medium, when culturing primate pluripotent stem cells. It was found that BME is one of various parameters in a culture medium that affects cloning efficiency of pluripotent cells in a positive manner. Specifically, when BME was either omitted or used in a concentration less than about 0.1 mM (between about 0 to about 0.1 mM) in the TeSR1 medium, the cells continued to proliferate (see FIG. 15a-b) with minimal to no change in the differentiation status of the cells (see FIG. 15c). Most notably, however, the cloning efficiency of the cells increased by at least 10% and preferably 20%, 30%, 40%, 50%, 60%, 70%, 80%, 90%, and 100% (see FIG. 15a) in comparison with pluripotent cells cultured in the same medium having higher than about 0.1 mM BME.

Accordingly, in one embodiment of the invention, a medium is disclosed for increasing the cloning efficiency of pluripotent cells in culture, wherein the medium contains salts, vitamins, amino acids, glucose, a fibroblast growth factor, less than about 0.1 mM beta-mercaptoethanol, and at least one member selected from gamma-aminobutyric acid, pipecolic acid, and lithium, in sufficient amounts to maintain stem cells grown in the medium in an undifferentiated state through multiple culture passages, wherein the cloning efficiency of the cells increases by at least 10% compared to the medium containing greater than or equal to 0.1 mM BME. In another embodiment, a method is disclosed for increasing cloning efficiency of cells in culture by at least 10% through culturing the primate pluripotent stem cells on a matrix in a medium without feeder cells or conditioned media, the medium comprising salts, vitamins, amino acids, glucose, a fibroblast growth factor, less than about 0.1 mM beta-mercaptoethanol and at least one member selected from gamma-aminobutyric acid, pipecolic acid, and lithium in sufficient amounts to maintain the cells in an undifferentiated state through multiple successive culture passages. In a related embodiment, when gamma-aminobutyric acid, pipecolic acid, lithium, and transforming growth factor beta are added in sufficient amounts to maintain the human stem cells in an undifferentiated state, at least 90% of the cells in culture are positive for the transcription factor Oct-4 through multiple successive culture passages. In general, the observation that human pluripotent stem cell cultures have previously been maintained in an undifferentiated state only when cultured in the presence of fibroblast feeder cells or in conditioned medium has led to speculation that the fibroblasts release a factor into the medium, which acts to inhibit human pluripotent stem cell differentiation. The data presented here demonstrate that this not the case. However, whatever effect fibroblast feeder cells have on culture medium, it is now clear that the media described below will substitute for that effect. The media described below, as defined, contain no non-human cells, and permit the long-term culture of undifferentiated human pluripotent stem cells. This strategy enables the preparation of a “humanized” medium and matrix to avoid any possible concerns about sub-cellular products of non-human origin.

According to some embodiments of the invention, the method of expanding and maintaining pluripotent stem cells in an undifferentiated state is effected by culturing the pluripotent stem cells in a culture medium being serum-free, feeder-free, matrix-free and protein carrier-free and comprising basic fibroblast growth factor (bFGF) at a concentration range of about 50-200 ng/ml.

According to some embodiments of the invention culturing is effected on a two-dimensional culture system such as a matrix or a feeder cell layer. For example, culturing on a two-dimensional culture system can be performed by plating the pluripotent stem cells onto a matrix or a feeder cell layer in a cell density which promotes cell survival and proliferation but limits differentiation. Typically, a plating density of between about 15,000 cells/cm2 and about 3,000,000 cells/cm2 is used. It will be appreciated that although single-cell suspensions of pluripotent stem cells are usually seeded, small clusters may also be used. To this end, enzymatic digestion (such as with type IV collagenase) utilized for cluster disruption is terminated before stem cells become completely dispersed and the cells are triturated with a pipette such that clumps (i.e., 10-200 cells) are formed. However, measures are taken to avoid large clusters which may cause cell differentiation. As used herein, the term “matrix” refers to any substance to which the pluripotent stem cells can adhere and which therefore can substitute the cell attachment function of feeder cells. Such a matrix typically contains extracellular components to which the pluripotent stem cells can attach and thus it provides a suitable culture substrate. According to some embodiments of the invention the matrix comprises an extracellular matrix.

The extracellular matrix can be composed of components derived from basemen membrane or extracellular matrix components that form part of adhesion molecule receptor-ligand couplings. MATRIGEL® (Becton Dickinson, USA) is one example of a commercially available matrix which is suitable for use with the present invention. MATRIGEL® is a soluble preparation from Engelbreth-Holm-Swarm tumor cells that gels at room temperature to form a reconstituted basement membrane; MATRIGEL® is also available as a growth factor reduced preparation. Other extracellular matrix components and component mixtures which are suitable for use with the present invention include foreskin matrix, laminin matrix, fibronectin matrix, proteoglycan matrix, entactin matrix, heparan sulfate matrix, collagen matrix and the like, alone or in various combinations thereof. According to some embodiments of the invention the matrix is xeno-free. In cases where complete animal-free culturing conditions are desired, the matrix is preferably derived from a human source or synthesized using recombinant techniques such as described hereinabove. Such matrices include, for example, human-derived fibronectin, recombinant fibronectin, human-derived laminin, foreskin fibroblast matrix or a synthetic fibronectin matrix. Human derived fibronectin can be from plasma fibronectin or cellular fibronectin, both of which can be obtained from Sigma, St. Louis, Mo., USA. Human derived laminin and foreskin fibroblast matrix can be obtained from Sigma, St. Louis, Mo., USA. A synthetic fibronectin matrix can be obtained from Sigma, St. Louis, Mo., USA. According to some embodiments of the invention, culturing is effected on a feeder cell layer. According to some embodiments of the invention, the method of expanding and maintaining pluripotent stem cells in an undifferentiated state is effected by culturing the pluripotent stem cells on a feeder cell layer in a serum-free and xeno-free culture medium which comprises basic fibroblast growth factor (bFGF), transforming growth factor beta-3 (TGFβ3) and ascorbic acid, wherein a concentration of the ascorbic acid in the culture medium is at least 50 μg/ml. According to some embodiments of the invention, the method of expanding and maintaining pluripotent stem cells in an undifferentiated state is effected by culturing the pluripotent stem cells on a feeder cell layer in a serum-free and xeno-free culture medium which comprises ascorbic acid at a concentration range of about 400-600 pg/ml, basic fibroblast growth factor (bFGF) at a concentration range of about 50-200 ng/ml, xeno-free serum replacement and a lipid mixture. According to some embodiments of the invention, the feeder cell layer is xeno-free. According to some embodiments of the invention, the feeder cell layer is a mesenchymal stem cell feeder cell layer. According to some embodiments of the invention, culturing according to some embodiments of the invention is effected in a suspension culture. According to some embodiments of the invention, the suspension culture is devoid of substrate adherence, e.g., without adherence to an external substrate such as components of extracellular matrix, a glass microcarrier or beads. According to some embodiments of the invention, culturing of the pluripotent stem cells in a suspension culture is effected in a protein carrier-free culture medium.

In some embodiments the iPSC cells generated for creation of “orthopedic relevant” mesenchymal stem cells require a “protein carrier”, this term refers to a protein which acts in the transfer of proteins or nutrients (e.g., minerals such as zinc) to the cells in the culture. Such protein carriers can be, for example, albumin (e.g., bovine serum albumin), Albumax (lipid enriched albumin) or plasmanate (human plasma isolated proteins). Since these carriers are derived from either human or animal sources their use in hESCs of human iPS cell cultures is limited by batch-specific variations and/or exposure to pathogens. Thus, a culture medium which is devoid of a protein carrier (e.g., albumin) is highly advantageous since it enables a truly defined medium that can be manufacture from recombinant or synthetic materials.

According to some embodiments of the invention, culturing of the pluripotent stem cells in a suspension culture is effected in a serum-free and feeder cell-free culture medium. It should be noted that some protocols of culturing pluripotent stem cells such as hESCs and iPS cells include microencapsulation of the cells inside a semipermeable hydrogel membrane, which allows the exchange of nutrients, gases, and metabolic products with the bulk medium surrounding the capsule (for details see e.g., U.S. Patent Application No. 20090029462 to Beardsley et al.). According to some embodiments of the invention, the pluripotent stem cells cultured in the suspension culture are devoid of cell encapsulation. According to an aspect of some embodiments of the invention, there is provided a method of expanding induced pluripotent stem (iPS) cells and maintaining the iPS cells in an undifferentiated state. The method is effected by culturing the iPS cells in a suspension culture under culturing conditions devoid of substrate adherence and devoid of cell encapsulation and which allow expansion of the iPS cells in the undifferentiated state. According to some embodiments of the invention, culturing of the pluripotent stem cells in a suspension culture is effected in the presence of the IL6RIL6 chimera-containing culture medium in which the concentration of the IL6RIL6 chimera is in the range of about 50-200 picograms per milliliter (pg/ml). According to some embodiments of the invention, culturing of the pluripotent stem cells in a suspension culture is effected in the presence of the leukemia inhibitory factor (LIF)-containing culture medium in which the concentration of LIF is at least about 2000 units/ml. According to some embodiments of the invention, culturing of the pluripotent stem cells in a suspension culture is effected in the presence of a medium which comprises basic fibroblast growth factor (bFGF) at a concentration range of about 50 ng/ml to about 200 ng/ml, e.g., between about 60 ng/ml to about 190 ng/ml, e.g., between about 70 ng/ml to about 180 ng/ml, e.g., between about 80 ng/ml to about 170 ng/ml, e.g., between about 90 ng/ml to about 160 ng/ml, e.g., between about 90 ng/ml to about 150 ng/ml, e.g., between about 90 ng/ml to about 130 ng/ml, e.g., between about 90 ng/ml to about 120 ng/ml, e.g., about 100 ng/ml. For example, a non-limiting example of a medium which was found suitable for culturing hESCs and human iPS cells in a suspension culture devoid of substrate adherence and cell encapsulation is the yF100 medium which comprises serum replacement and 100 ng/ml bFGF. According to some embodiments of the invention, culturing of the pluripotent stem cells in a suspension culture is effected in the presence of a medium which comprises the IL6RIL6 chimera at a concentration range of about 50-200 nanogram per milliliter (ng/ml) and bFGF at a concentration in the range of 1-50 ng/ml. For example, a non-limiting example of a medium which was found suitable for culturing hESCs and human iPS cells in a suspension culture devoid of substrate adherence and cell encapsulation is the CM100F medium which comprises serum replacement, the IL6RIL6 chimera at a concentration of 100 ng/ml and bFGF at a concentration of 10 ng/ml.

Culturing in a suspension culture according to the method of some embodiments of the invention is effected by plating the pluripotent stem cells in a culture vessel at a cell density which promotes cell survival and proliferation but limits differentiation. Typically, a plating density of between about 5×104-2×106 cells per ml is used. It will be appreciated that although single-cell suspensions of stem cells are usually seeded, small clusters such as 10-200 cells may also be used.

In order to provide the pluripotent stem cells with sufficient and constant supply of nutrients and growth factors while in the suspension culture, the culture medium can be replaced on a daily basis, or, at a pre-determined schedule such as every 2-3 days. For example, replacement of the culture medium can be performed by subjecting the pluripotent stem cells suspension culture to centrifugation for about 3 minutes at 80 g, and resuspension of the formed pluripotent stem cells pellet in a fresh medium. Additionally or alternatively, a culture system in which the culture medium is subject to constant filtration or dialysis so as to provide a constant supply of nutrients or growth factors to the pluripotent stem cells may be employed.

Since large clusters of pluripotent stem cells may cause cell differentiation, measures are taken to avoid large pluripotent stem cells aggregates. According to some embodiments of the invention, the formed pluripotent stem cells clumps are dissociated every 5-7 days and the single cells or small clumps of cells are either split into additional culture vessels (i.e., passaged) or remained in the same culture vessel yet with additional culture medium. For dissociation of large pluripotent stem cells clumps, a pellet of pluripotent stem cells (which may be achieved by centrifugation as described hereinabove) or an isolated pluripotent stem cells clump can be subject to enzymatic digestion and/or mechanical dissociation. To maintain human pluripotent cells in an undifferentiated state, cultures must provide the cells with conditions which maintain cell proliferation, inhibit cell differentiation and preserve pluripotency. Such culturing conditions are typically achieved by utilizing feeder cell layers which secrete factors needed for stem cell proliferation, while at the same time, inhibit their differentiation. In order to traverse limitations associated with feeder cell layer use such as feeder cells contamination and undefined culture systems, more defined feeder cell-free culture systems have been developed. Feeder cell-free culture systems employ a matrix, which the pluripotent cells are attached thereto, and a culture medium, which provides the ES cells with cytokines and growth factors needed for cell proliferation, while at the same time inhibits cell differentiation. Commonly used matrices include the basement membrane preparation extracted from Engelbreth-Holm-Swarm (EHS) mouse sarcoma (e.g., Matrigel™), or bovine-fibronectin/laminin. Such matrices are usually supplemented with a mouse embryonic fibroblast (MEF) conditioned medium, or a synthetic medium supplemented with bovine serum and growth factors. Previous attempts to culture human ES cells using feeder cells-free culture systems employed Matrigel™ or laminin matrices supplemented with fresh culture medium and a growth factor mixture (U.S. Pat. Appl. No. 20030017589). However, these feeder cells-free matrices were derived from animal tissues and therefore may expose the human ES cells to animal pathogens. In addition, these experiments used a combination of six different growth factors at extremely high concentrations which may irreversibly damage the cultured cells. Indeed, as is demonstrated in U.S. Pat Appl. No. 20030017589, the doubling time of the ES cells was approximately 19 hours, suggesting a tumorigenic phenotype. Moreover, under these conditions only 50-70% of the cells exhibited an undifferentiated cell morphology following 14 passages on feeder cells-free culture systems. Although such culturing conditions might be suitable for research purposes, human ES cells must be cultured under well-defined culture conditions which are essentially free of animal material when utilized for cell replacement therapy or tissue regeneration in humans. While reducing the present invention to practice, the present inventors have devised feeder cell-free culturing conditions which are devoid of xeno-contaminants and yet are capable of sustaining human stem cells in culture for at least 38 passages. As is illustrated in the Examples section which follows, stem cell lines cultured under such conditions maintained all cell features including pluripotency, immortality, undifferentiated proliferation capacity and normal karyotype. Thus, the feeder cells-free culture system of the present invention provides, for the first time, a complete animal-free culturing environment, which is capable of maintaining human cells for at least 38 passages in a proliferative state while preserving pluripotency. In addition, more than 85% of ES cells cultured under such conditions exhibited undifferentiated cell morphology with a doubling time of 30-35 hours. Thus, according to the present invention there is provided a method of establishing a human pluripotent stem cell line capable of being maintained in an undifferentiated, pluripotent and proliferative state and being substantially free of xeno-contaminants According to one aspect of the present invention, the method is effected by obtaining human embryonic stem cells and culturing the human stem cells under feeder cells-free culturing conditions which include a matrix and a tissue culture medium including growth factors to thereby establish a human embryonic stem cell line. According to this aspect of the present invention, culturing is effected by plating the stem cells onto a matrix in a cell density which promotes cell survival and proliferation but limits differentiation. Typically, a plating density of between about 15,000 cells/cm2 and about 200,000 cells/cm2 is used. It will be appreciated that although single-cell suspensions of stem cells are usually seeded, small clusters may also be used. To this end, enzymatic digestion utilized for cluster disruption is terminated before stem cells become completely dispersed and the cells are triturated with a pipette such that clumps (i.e., 10-200 cells) are formed. However, measures are taken to avoid large clusters which cause cell differentiation.

The stem cells of the present invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell can be expanded to the blastocyst stage. For the isolation of human cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; Gardner et al., [Fertil. Steril. 69: 84, 1998]. It will be appreciated that commercially available stem cells can also be used with this aspect of the present invention. Human ES cells can be purchased from the NIH human embryonic stem cells registry (http://escr.nih.gov). Non-limiting examples of commercially available embryonic stem cell lines are BG01, BG02, BG03, BG04, CY12, CY30, CY92, CY10, TE03 and TE32. Stem cells used by the present invention can be also derived from human embryonic germ (EG) cells. Human EG cells are prepared from the primordial germ cells obtained from human fetuses of about 8-11 weeks of gestation using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622. As is mentioned hereinabove, the stem cells can are preferably cultured on a feeder cells-free culture system which includes a matrix instead of a feeder cell layer. As used herein, the term “matrix” refers to any matrix which can substitute the cell attachment function of feeder cells. Such a matrix typically contains extracellular components to which the stem cells can attach and thus it provides a suitable culture substrate. Particularly suitable for use with the present invention are extracellular matrix components derived from basement membrane or extracellular matrix components that form part of adhesion molecule receptor-ligand couplings. Matrigel® is one example of a commercially available matrix (Becton Dickinson, USA) which is suitable for use with the present invention. Matrigel® is a soluble preparation from Engelbreth-Holm-Swarm tumor cells that gels at room temperature to form a reconstituted basement membrane; Matrigel® is also available as a growth factor reduced preparation. Other extracellular matrix components and component mixtures which are suitable for use with the present invention include laminin, fibronectin, proteoglycan, entactin, heparan sulfate, and the like, alone or in various combinations. Preferred matrices of the present invention are fibronectin derived matrices. In cases where complete animal-free culturing conditions are desired, the matrix is preferably derived from a human source or synthesized using recombinant techniques. Such matrices include, for example, human-derived fibronectin recombinant fibronectin, human-derived laminin, foreskin fibroblast matrix or a synthetic fibronectin matrix. Human derived fibronectin can be from plasma fibronectin or cellular fibronectin, both of which can be obtained from Sigma, St. Louis, Mo., USA. Human derived laminin and foreskin fibroblast matrix can be obtained from Sigma, St. Louis, Mo., USA. A synthetic fibronectin matrix can be obtained from Sigma, St. Louis, Mo., USA. Recombinant synthesis of matrix proteins can be effected by using expression vectors. The polynucleotide segments encoding the matrix protein (e.g., human plasma fibronectin) can be ligated into a commercially available expression vector system suitable for transforming mammalian cells such as HeLa cells and for directing the expression of this enzyme within the transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

Suitable mammalian expression vectors include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Strategene, pTRES which is available from Clontech, and their derivatives. According to preferred embodiments of the present invention, the culture medium includes cytokines and growth factors needed for cell proliferation [e.g., basic fibroblast growth factor (bFGF) and leukemia inhibitor factor (LIF)], and factors such as transforming growth factor β1 (TGFβ 1) which inhibit stem cell differentiation. Such a culture medium can be a synthetic tissue culture medium such as Ko-DMEM (Gibco-Invitrogen Corporation products, Grand Island, N.Y., USA) supplemented with serum, serum replacement and/or growth factors. Serum can be of any source including fetal bovine serum, goat serum or human serum. Preferably human serum or serum Replacement™ (Gibco-Invitrogen Corporation, Grand Island, N.Y. USA) are utilized in order to provide an animal-free environment for the human ES cells.

Serum Replacement™ includes albumin or albumin substitutes, amino acids, vitamins, transferrins or transferrin substitutes, antioxidants, insulin or insulin substitutes, collagen precursors and trace elements (International Patent Publication No. WO 98/30679 to Price, P. J. et al). To provide animal-free culture conditions the albumin or albumin substitutes are preferably derived from a human source and/or are recombinant proteins. Culture medium, serum, and serum replacement can be obtained from any commercial supplier of tissue culture products, examples include Gibco-Invitrogen Corporation (Grand Island, N.Y. USA), Sigma (St. Louis Mo., USA) and the ATCC (Manassas, Va. USA). The serum or serum replacement used by the present invention are provided at a concentration range of 1% to 40%, more preferably, 5% to 35%, most preferably, 10% to 30%. Growth factors of the present invention can be used at any combination and can be provided to the stem cells at any concentration suitable for cell proliferation, while at the same time inhibit cell differentiation. Suitable growth factors according to the present invention include, but are not limited to, transforming growth factor 31 (TGFβ 1), basic fibroblast growth factor (bFGF) and human recombinant leukemia inhibitor factor (LIF), ciliary neurotrophic factor (CNTF), recombinant human Oncostatin M, interleukin 6 (IL-6) Flt-3 ligand, stem cell factor (SCF) and the like. Such growth factors can be obtained from any supplier of tissue culture reagents such as Gibco Invitrogen Corporation Products, USA, R & D Systems Inc. Minneapolis, Minn., USA and Chemicon International Inc., Temecula, Calif., USA.

Enzymatic digestion of pluripotent stem cells clump(s) can be performed by subjecting the clump(s) to an enzyme such as type IV Collagenase (Worthington biochemical corporation, Lakewood, N.J., USA) and/or Dispase (Invitrogen Corporation products, Grand Island N.Y., USA). The time of incubation with the enzyme depends on the size of cell clumps present in the suspension culture. Typically, when pluripotent stem cells cell clumps are dissociated every 5-7 days while in the suspension culture, incubation of 20-60 minutes with 1.5 mg/ml type IV Collagenase results in small cell clumps which can be further cultured in the undifferentiated state. Alternatively, pluripotent stem cells clumps can be subjected to incubation of about 25 minutes with 1.5 mg/ml type IV Collagenase followed by five minutes incubation with 1 mg/ml Dispase. It should be noted that passaging of human ESCs with trypsin may result in chromosomal instability and abnormalities (see for example, Mitalipova M M., et al., Nature Biotechnology, 23: 19-20, 2005 and Cowan C A et al., N. Engl. J. of Med. 350: 1353-1356, 2004). According to some embodiments of the invention, passaging hESC or iPS cell with trypsin should be avoided. Mechanical dissociation of large pluripotent stem cells clumps can be performed using a device designed to break the clumps to a predetermined size. Such a device can be obtained from CellArtis Goteborg, Sweden. Additionally or alternatively, mechanical dissociation can be manually performed using a needle such as a 27 g needle (BD Microlance, Drogheda, Ireland) while viewing the clumps under an inverted microscope. According to some embodiments of the invention, following enzymatic or mechanical dissociation of the large cell clumps, the dissociated pluripotent stem cells clumps are further broken to small clumps using 200 μl Gilson pipette tips (e.g., by pipetting up and down the cells). The culture vessel used for culturing the pluripotent stem cells in suspension according to the method of some embodiments of the invention can be any tissue culture vessel (e.g., with a purity grade suitable for culturing pluripotent stem cells) having an internal surface designed such that pluripotent stem cells cultured therein are unable to adhere or attach to such a surface (e.g., non-tissue culture treated cells, to prevent attachment or adherence to the surface). Preferably, in order to obtain a scalable culture, culturing according to some embodiments of the invention is effected using a controlled culturing system (preferably a computer-controlled culturing system) in which culture parameters such as temperature, agitation, pH, and pO2 is automatically performed using a suitable device. Once the culture parameters are recorded, the system is set for automatic adjustment of culture parameters as needed for pluripotent stem cells expansion.

One method of increasing the efficacy of pluripotent stem cell proliferation is to reduce cell to cell adhesion. In some embodiments the reduction of cell to cell adhesion is performed by addition of an inhibitor substance. Such an inhibitory substance can be hemagglutinin (HA) of the neurotoxin complex of Clostridium botulinum, in one or more non-limiting embodiments. In the practice of the invention, the substance that can inhibit cell-cell adhesion is a complex composed of two or three components selected from the group consisting of three hemagglutinin subcomponents HA1 (HA33), HA2 (HA17), and HA3 (HA70) of the neurotoxin complex of Clostridium botulinum, or a substance containing the complex, in one or more non-limiting embodiments. Further, from the viewpoint of efficiently removing deviated cells, the substance that can inhibit cell-cell adhesion is a complex composed of HA2 (HA17) and HA3 (HA70), a complex composed of the three components, or a substance containing the complex, in one or more non-limiting embodiments. From the viewpoint of causing the activity of inhibiting E-cadherin function to be expressed, and from the viewpoint of efficiently removing deviated cells, the subcomponent HA3 (HA70) is preferably of Clostridium botulinum type A or Clostridium botulinum type B, in one or more embodiments. Further, the subcomponents HA1 (HA33) and HA2 (HA17) may be of any one of Clostridium botulinum type A, Clostridium botulinum type B, and Clostridium botulinum type C, in one or more non-limiting embodiments. Regarding HA, each subcomponent may be of a recombinant type or a natural type, in one or more non-limiting embodiments. In the present disclosure, “cell culture in the presence of the substance that can inhibit cell-cell adhesion” can use culture conditions, a culture medium, and the like that are conventionally used and/or will be developed in future for stem cells having pluripotency, and this can be achieved by making the substance that can inhibit cell-cell adhesion be present in the medium under the culture conditions. In one or more non-limiting embodiments, the substance that can inhibit cell-cell adhesion may be added to a culture medium under culture, or alternatively, a medium to which the substance that can inhibit cell-cell adhesion is preliminarily added may be used for culture. As the culture medium, the culture plate, and the like, those which are commercially available may be used. The “substance that can inhibit cell-cell adhesion” may be added to a medium after deviated cells are confirmed, or alternatively, may be added to a medium at a stage where deviated cells have not emerged yet. The concentration of the “substance that can inhibit cell-cell adhesion” present in a medium is a substantially effective concentration that enables removal of deviated cells, in one or more non-limiting embodiments, and any person skilled in the art is able to set the concentration. From the viewpoint of efficiently removing deviated cells, the concentration of the “substance that can inhibit cell-cell adhesion” present in the medium is 5 nM or more, 10 nM or more, or alternatively, 15 nM or more, for example, in one or more non-limiting embodiments. From the same viewpoint, the concentration is 200 nM or less, 150 nM or less, or alternatively, 100 nM or less. In one or more non-limiting embodiments in which the “substance that can inhibit cell-cell adhesion” is present in the medium, the administration of the same may be a single administration per one period, which is until next medium exchange, or serial administration, or alternatively, occasional administration. In the present disclosure, cell culture includes subculture, in one or more non-limiting embodiments. By the culturing method according to the present disclosure, and/or according to the removing method according to the present disclosure, in one aspect, an effect can be achieved that the ratio of an undifferentiated colony (a colony that is formed with undifferentiated cells and that substantially does not contain deviated cells) in a colony formed after subculture can be improved. The subculture can be performed by any of techniques that are conventionally known and are to be developed in future, in one or more non-limiting embodiments.

In the present disclosure, the cell culture may be culture using feeder cells, or may be feeder-free culture, in one or more non-limiting embodiments. Examples of the feeder cells include MEF (Mouse Embryo Fibroblast) cells, SL10, and SNL 76/7 feeder cells, in one or more non-limiting embodiments. Among the feeder cells, feeder cells that allow the migration speed of stem cells having pluripotency to be relatively slow are preferred, in one or more non-limiting embodiments. In one or more non-limiting embodiments, the feeder cells are preferably SNL 76/7 feeder cells, from the viewpoint that the migration of stem cells having pluripotency is relatively slow and a colony of deviated cells is allowed to emerge in the center part of a colony during culture of stem cells having pluripotency. In other embodiments the feeder cells are mesenchymal stem cell that are sourced from primary sources or that are immortalized. The mesenchymal stem cells may be selected for particular immature phenotypes.

The cell culture is preferably performed under conditions in which deviated cells may possibly emerge in a center part of a colony during culture of stem cells having pluripotency, from the viewpoint that the deviated cells can be removed efficiently, in one or more non-limiting embodiments. In one or more non-limiting embodiments, the migration of stem cells having pluripotency is inhibited and/or suppressed, whereby deviated cells can efficiently emerge in the center part of the colony. In another embodiment, the present disclosure relates to a method for culturing stem cells having pluripotency, the method including culturing cells in the presence of a substance that can inhibit migration, and performing cell culture in the presence of a substance that can inhibit cell-cell adhesion. By the culturing method according to the present aspect, in one aspect, deviated cells are allowed to emerge in the center part of a colony during culture of stem cells having pluripotency, and this makes it possible to achieve an effect of efficiently removing deviated cells. The present disclosure, in another aspect, relates to a method for removing deviated cells that have emerged or may possibly emerge during culture of stem cells having pluripotency, the method including culturing cells in the presence of a substance that can inhibit migration, and performing cell culture in the presence of a substance that can inhibit cell-cell adhesion. By the culturing method according to the present aspect, and/or by the removing method according to the present aspect, in one aspect, deviated cells can be removed from a colony of cells in the undifferentiated state in which deviated cells emerge and that therefore deteriorates, whereby a colony of cells in the undifferentiated state or a colony composed of cells in the undifferentiated state can be obtained. The present disclosure, in another aspect, relates to a method for forming a colony composed of cells in the undifferentiated state out of a colony where deviated cells emerge and that therefore deteriorates, the method including; culturing cells in the presence of a substance that can inhibit migration; and culturing the deteriorated colony in the presence of a substance that can inhibit cell-cell adhesion. In the present disclosure, in one or more non-limiting embodiments, examples of the substance that can inhibit migration include a substance that suppresses/inhibits activity of a substance relating to migration of stem cells having pluripotency. In the present disclosure, in one or more non-limiting embodiments, examples of the substance that can inhibit migration include a migration inhibitor. In one or more non-limiting embodiments, examples of the substance that can inhibit migration include a Rac-1 inhibitor. In one or more non-limiting embodiments, from the viewpoint of efficiently moving deviated cells to the center part of a colony and efficiently removing the deviated cells, the concentration of the “substance that can inhibit migration” that is caused to be present in the medium is 50 μM or more, 100 μM or more, or alternatively, 150 μM or more. From the same viewpoint, the concentration is 200 μM or less.

For non-dynamic culturing of pluripotent stem cells, the pluripotent stem cells can be cultured in uncoated 58 mm Petri dishes (Greiner, Frickenhausen, Germany). For dynamic culturing of pluripotent stem cells, the pluripotent stem cells can be cultured in spinner flasks [e.g., of 200 ml to 1000 ml, for example 250 ml which can be obtained from CellSpin of Integra Biosciences, Fernwald, Germany; of 100 ml which can be obtained from Bellco, Vineland, N.J.; or in 125 ml Erlenmeyer (Corning Incorporated, Corning N.Y., USA)] which can be connected to a control unit and thus present a controlled culturing system. The culture vessel (e.g., a spinner flask, an Erlenmeyer) is shaken continuously. According to some embodiments of the invention the culture vessels are shaken at 90 rounds per minute (rpm) using a shaker (S3.02.10L, ELMI Itd, Riga, Latvia). According to some embodiments of the invention the culture medium is changed daily. According to some embodiments of the invention, when cultured according to the teachings of the present invent ion, the growth of the pluripotent stem cells is monitored to determine their differentiation state. The differentiation state can be determined using various approaches including, for example, morphological. Determination of ES cell differentiation can also be affected via measurements of alkaline phosphatase activity. Undifferentiated human ES cells have alkaline phosphatase activity which can be detected by fixing the cells with 4% paraformaldehyde and developing with the Vector Red substrate kit according to manufacturer's instructions (Vector Laboratories, Burlingame, Calif., USA). The present inventors have uncovered that the novel xeno-free and serum free culture media of the invention can be used to derive new pluripotent stem cell lines.

1. A mesenchymal stem cell useful for the treatment of orthopedic conditions, wherein said mesenchymal stem cell is: a) generated from a pluripotent stem cell; b) possess enhanced regenerative activity; and c) optionally possesses enhanced antioxidant activity.

2. The mesenchymal stem cell of claim 1, wherein said orthopedic condition is selected from the group consisting of: a) bone fracture; b) non-union bone fracture; c) osteoarthritis; d) rheumatoid arthritis; e) cartilage degeneration; f) torn meniscus; and g) degenerative disc disease.

3. The mesenchymal stem cell of claim 2, wherein said osteoarthritis is associated with increased expression of MMP-1.

4. The mesenchymal stem cell of claim 1, wherein said cell is generated by culture of pluripotent stem cells in a decellularized bone matrix.

5. The mesenchymal stem cell of claim 4, wherein said pluripotent stem cell is first cultured in a suspension culture, wherein said suspension culture allows for said pluripotent stem cells to form embryoid bodies.

6. The mesenchymal stem cell of claim 5, wherein said suspension culture is performed under conditions of hypoxia.

7. The mesenchymal stem cell of claim 6, wherein said hypoxia is performed for a time period and intensity sufficient to allow for nuclear translocation of hypoxia inducible factor.

8. The mesenchymal stem cell of claim 1, wherein said pluripotent stem cell is a stressed induced dedifferentiated stem cell.

9. The mesenchymal stem cell of claim 1, wherein said pluripotent stem cell is an induced pluripotent stem cell.

10. The mesenchymal stem cell of claim 9, wherein said induced pluripotent stem cells are generated from mesenchymal stem cells.

11. The mesenchymal stem cell of claim 10, wherein said mesenchymal stem cell is purified from perinatal tissue.

12. The mesenchymal stem cell of claim 1, wherein said cell is engineered to possess increased expression of bone morphogenic protein 2 as compared to a non-engineered mesenchymal stem cell.

13. The mesenchymal stem cell of claim 1, wherein said cell is utilized to enhance engraftment of a chondrocytic progenitor.

14. The mesenchymal stem cell of claim 13, wherein said chondrocytic progenitor is allogeneic to the recipient.

15. The mesenchymal stem cell of claim 13, wherein said chondrocytic progenitor is utilized to treat a defect of hyalin cartilage.

16. The mesenchymal stem cell of claim 1, wherein said cell is utilized to treat a non-union bone fracture.

17. The mesenchymal stem cell of claim 16, wherein said mesenchymal stem cell is administered together with an anti-inflammatory agent.

18. The mesenchymal stem cell of claim 17, wherein said anti-inflammatory agent is capable of inhibiting activation of NF-kappa B.

19. The mesenchymal stem cell of claim 17, wherein said anti-inflammatory agent is n-acetylcysteine.

20. The mesenchymal stem cell of claim 19, wherein said quercetin is administered at a concentration sufficient to increase production of IL-10 from mesenchymal stem cells stimulated with HMGB1 by over 25% as compared to baseline.
